(19) United States
(12) Patent Application Publication
(10) Pub. No.: US 2003/0138800 A1
(43) Pub. Date: Jul. 24, 2003

(54) Exponential amplification of nucleic acids using nicking agents

(75) Inventors: Jeffrey Van Ness, Claremont, CA (US); David J. Galas, Claremont, CA (US); Lori K. Van Ness, Claremont, CA (US)

Correspondence Address: SEED INTELLECTUAL PROPERTY LAW GROUP PLLC, 701 FIFTH AVE, SUITE 6300, SEATTLE, WA, 98104-7092, US

(73) Assignee: Keck Graduate Institute, Claremont, CA (US)

(21) Appl. No.: 10/196,740

(22) Filed: Jul. 15, 2002

Related U.S. Application Data
(60) Provisional application No. 60/345,445, filed on Jan. 2, 2002.
(60) Provisional application No. 60/331,687, filed on Nov. 19, 2001.
(60) Provisional application No. 60/305,637, filed on Jul. 15, 2001.

(51) Int. Cl. C12Q 1/68; C12P 19/34

(52) U.S. Cl. 435/6; 435/91.2

(57) ABSTRACT

The present invention provides methods and compositions for exponential amplification of nucleic acid molecules using nicking agents. In certain aspects, the amplification may be performed isothermally. This invention is useful in many areas such as disease diagnosis.

### BACKGROUND OF THE INVENTION

1. Field of the Invention

This invention relates to the field of molecular biology, more particularly to methods and compositions involving nucleic acids, and still more particularly to methods and compositions related to amplifying nucleic acids using a nicking agent.

2. Description of the Related Art

A number of methods have been developed for rapid amplification of nucleic acids. These include the polymerase chain reaction (PCR), ligase chain reaction (LCR), self-sustained sequence replication (3SR), nucleic acid sequence based amplification (NASBA), transcription-based amplification system (TAS), strand displacement amplification (SDA), and amplification with Qβ replicase. Most of the methods widely used for nucleic acid amplification, such as PCR, require cycles of different temperatures to achieve cycles of denaturation and reannealing. Other methods, although they may be performed isothermally, require multiple sets of primers (e.g., bumper primers of thermophilic SDA) or are based on transcription and/or reverse transcription, which is sensitive to RNA degradation (e.g., TAS, NASBA and 3SR). Accordingly, there is a long felt need in the art for a simpler and more efficient method for nucleic acid amplification.

The present invention fulfills this and related needs as described below.

### BRIEF SUMMARY OF THE INVENTION

In contrast to previously known techniques for amplification of nucleic acids, the present invention provides a method for nucleic acid amplification that does not require the use of multiple sets of oligonucleotide primers and is not transcription-based. In addition, the present invention can be carried out under an isothermal condition, thus avoiding the expenses associated with the equipment for providing cycles of different temperatures. The present invention may find utilities in various applications such as disease diagnosis.

In one aspect, the present invention provides methods for amplifying a nucleic acid molecule (where this amplified molecule is often referred to herein as “A2”). In one aspect, a method of the present invention includes: (A) Providing a nucleic acid molecule (referred to as “N1”) that is at least partially double-stranded, and may be completely double-stranded, where N1 includes one or both of (i) and (ii), where (i) is the nucleotide sequence of the sense strand of a first nicking agent recognition sequence (NARS), and (ii) is the nucleotide sequence of the antisense strand of the first NARS. (B) Amplifying a first single-stranded nucleic acid molecule (referred to herein as “A1”) in the presence of a first nicking agent (NA) that recognizes the first NARS, a DNA polymerase, and one or more deoxynucleoside triphosphate(s). This amplification uses a portion of N1 as a template for the polymerase. (C) Providing a second single-stranded nucleic acid molecule (referred to as “T2”). T2 includes, as seen sequentially from the 5′ end to the 3′ end of T2, each of (i), (ii) and (iii), where (i) is a template nucleotide sequence, (ii) is the nucleotide of the antisense strand of a second NARS, and (iii) is a nucleotide sequence that is at least substantially, and optionally is exactly, complementary to A1. (D) Amplifying a third single-stranded nucleic acid molecule (referred to as “A2”) in the presence of T2, A1, the first NA, a second NA that recognizes the second NARS, a DNA polymerase and deoxynucleoside triphosphate(s), where A2 is complementary to at least a portion of the template nucleotide sequence of T2. In optional embodiments of this method of the invention for amplifying a nucleic acid molecule, any one, any two, any three, any four, any five, any six, any seven, or any eight, or any nine, or any ten, or all eleven of the following criteria (a) through (k) may be used in further describing the method: (a) N1 is a partially double-stranded nucleic acid molecule, (b) N1 includes the nucleotide sequence of the sense strand of the first NARS, (c) N1 includes the nucleotide sequence of the antisense strand of the first NARS, (d) N1 includes a 5′ overhang in the strand that contains the NS of the first NA, (e) N1 includes a 5′ overhang in the strand that is extended according to the present invention to include the NS of the first NA, (f) N1 includes a 3′ overhang in the strand that does not contain the NS of the first NA, (g) N1 includes a 3′ overhang in the strand that is not extended according to the method of the present invention to provide a NS for the first NA, (h) N1 includes a 5′ overhang that includes a nucleotide sequence that is at least substantially complementary to, and optionally is exactly complementary to, a nucleotide sequence in a target nucleic acid, (i) N1 includes a 3′ overhang that includes a nucleotide sequence that is at least substantially complementary to, and optionally is exactly complementary to, a nucleotide sequence in a target nucleic acid, (j) N1 includes a nucleotide sequence within the strand that does not get nicked by the first nicking agent, and more specifically is located 5′ to the position corresponding to the NS, where this nucleotide sequence functions as a template for amplifying A1, (k) N1 includes a nucleotide sequence within the strand that does not get extended according to the present invention to provide a site that gets nicked by the first nicking agent, where this nucleotide sequence is located 5′ to the position corresponding to the NS and this nucleotide sequence functions as a template for amplifying A1, so that for example the present invention provides the method for amplifying a nucleic acid molecule as outlined above wherein: N1 is a partially double-stranded nucleic acid molecule that includes: (1) the nucleotide sequence of the sense strand of the first NARS, the nucleotide sequence of the antisense strand of the first NARS, or both of these sequences, (2) either a 5′ overhang in the strand that either contains the NS that gets nicked by the first NA or is extended according to the present invention to provide the NS that gets nicked by the first NA, or a 3′ overhang in the strand that does not contain the NS that gets nicked by the first NA, and is not extended according to the present invention to provide the NS for the first NA, wherein the 5′ or 3′ overhang (whichever is/are present) includes a nucleotide sequence that is at least substantially complementary to, and optionally is exactly complementary to, a target nucleic acid, and (3) a nucleotide sequence within the strand that does not contain the NS that gets nicked by the first NA, and is not extended according to the present invention to provide a NS for the first NA, where this nucleotide sequence is located 5′ to the position corresponding to the NS and this nucleotide sequence functions as a template for amplifying A1.

In another aspect, the present invention provides a method for amplifying a nucleic acid molecule (this amplified molecule being referred to as “A2”), where this method includes: (A) Forming a mixture that includes (i), (ii), and (iii), where (i) is an at least partially double-stranded nucleic acid molecule (referred to herein as “N1”) that includes the nucleotide sequence of the antisense strand of a first nicking agent recognition sequence (NARS), (ii) is a single-stranded nucleic acid molecule (referred to herein as “T2” that includes, as viewed from the 5′ direction of the molecule to the 3′ direction, the components (a), (b) and (c), where (a) is a template nucleotide sequence, (b) is the nucleotide sequence of the antisense strand of a second NARS, and (c) is a nucleotide sequence that is at least substantially identical to, and optionally is exactly identical to, a portion of N1 located 5′ to the antisense strand of the NARS in N1, and (iii) is a first nicking agent (NA) that recognizes the first NARS, a second NA that recognizes the second NARS, a DNA polymerase, and one or more deoxynucleoside triphosphate(s). (B) Maintaining the mixture of (A) under conditions that (1) amplify a single-stranded nucleic acid molecule (referred to herein as “A1”) using a portion of N1 as a template and (2) amplify a single-stranded nucleic acid molecule (referred to herein as “A2”) using the template nucleotide sequence of T2 as a template. As one way to prepare N1, there is provided a method whereby a trigger oligonucleotide primer (ODNP) is annealed to a single-stranded target nucleic acid (referred to herein as “T1”), where T1 includes, as viewed from a 5′ direction of the molecule to a 3′ direction: (1) the nucleotide sequence of the antisense strand of the first NARS; and (2) a nucleotide sequence that is at least substantially complementary to, and optionally is exactly complementary to, at least a portion of the trigger ODNP. In one embodiment of the invention, the mixture formed in (A) further includes (iv) a single-stranded nucleic acid molecule (referred to herein as “T3”) where T3 includes, as viewed from a 3′ direction to a 5′ direction, the components (a), (b) and (c) where (a) is a nucleotide sequence that is at least substantially identical to, and optionally is exactly identical to, at least a portion of the template nucleotide sequence of T2, (b) is the nucleotide sequence of the antisense strand of a third NARS, and (c) is a second template nucleotide sequence. Optionally, said mixture (A), in the event it contains component (iv), is maintained under conditions that amplify a single-stranded nucleic acid molecule (A3) that includes a nucleotide sequence that is complementary to at least a portion of the nucleotide sequence that forms the second template T3.

In a related aspect, the present invention provides a method for amplifying a nucleic acid molecule (A2), where this method includes: (A) Providing a template nucleic acid molecule (T2) that can hybridize to A2. (B) Providing a primer nucleic acid molecule (A1) that can hybridize to T2 at a location on T2 that is 3′ of the location where A2 can hybridize to T2. (C) Hybridizing A1 to T2. (D) Extending A1 to provide an A1 extension product, where the A1 extension product when hybridized to T2 forms a hybrid H2 that comprises a second nicking agent recognition sequence (NARS) and the nucleotide sequence of A2. (E) Nicking H2 with a second nicking agent (NA) that recognizes the second NARS to thereby form A2. (F) Repeating steps (E) and (E) to thereby amplify A2. Preferably, the primer nucleic acid molecule A1 is formed by a method that includes: (G) Providing a template nucleic acid molecule (T1) that can hybridize to A1. (H) Providing a trigger oligonucleotide primer (ODNP) that can hybridize to T1 at a location on T1 that is 3′ of the location where A1 can hybridize to T1. (I) Hybridizing the trigger ODNP to T1. (J) Extending the trigger ODNP to provide a trigger ODNP extension product, where the trigger ODNP extension product, when hybridized to T1, forms a hybrid H1 that comprises a first NARS and the nucleotide sequence of A1. (K) Nicking H1 with a first NA that recognizes the first NARS to thereby form A1.

In another aspect, the present invention provides a method for amplifying a nucleic acid molecule (referred to herein as “A2”), where the method includes: (A) Forming a mixture of (i), (ii) and (iii), where (i) is an at least partially double-stranded, and may be a fully double-stranded, nucleic acid molecule (referred to herein as “N1”) that includes the nucleotide sequence of the sense strand of a first nicking endonuclease recognition sequence (NERS), (ii) is a single-stranded nucleic acid molecule (T2) that includes (a), (b) and (c) which are, from 3′ to 5′ (i.e., the following nucleotide sequences (a), (b) and (c) are present in T2 in the stated order 3′(a)(b)(c)₅′): (a) a nucleotide sequence that is at least substantially complementary, and optionally exactly complementary, to a portion of the nucleotide sequence of N1 that is located 3′ to the sense strand of the NERS in N1, (b) the nucleotide sequence of the antisense strand of a second NERS, and (c) a template nucleotide sequence; and (iii) is a first nicking endonuclease (NE) that recognizes the first NERS, a second NE that recognizes the second NERS, a DNA polymerase, and one or more deoxynucleoside triphosphate(s). (B) Maintaining the mixture (A) under conditions that amplify a single-stranded nucleic acid molecule (A2) using the template nucleotide sequence of T2 as a template. Optionally, N1 is provided by annealing a trigger oligonucleotide primer (ODNP) to a single-stranded target nucleic acid (T1), where T1 includes, as viewed from a 5′ to 3′ direction of T1, both of (A) and (B), where (A) is the nucleotide sequence of the sense strand of the first NERS, and (B) is a nucleotide sequence that is at least substantially complementary, and optionally is exactly complementary, to at least a portion of the trigger ODNP. Also optionally, the mixture (A) further includes component (iv), where (iv) is a single-stranded nucleic acid molecule (T3) that includes, from 3′ to 5′, each of (a), (b) and (c), where (a) is a nucleotide sequence that is at least substantially identical, and optionally is identical, to at least a portion of the template nucleotide sequence of T2, (b) is the nucleotide sequence of the antisense strand of the NERS; and (c) is a second template nucleotide sequence. Optionally, when the mixture (A) contains component (iv), the method includes maintaining mixture (A) under conditions that amplify a single-stranded nucleic acid molecule (referred to herein as “A3”) that is complementary to at least a portion of the second template nucleotide sequence of T3. Optionally, T3 has a nucleotide sequence that is exactly identical to at least a portion of the template nucleotide sequence of T2.

In another aspect, the present invention provides a method of amplifying a nucleic acid (A2), where the method includes: (A) Providing a first template nucleic acid molecule (T1) that comprises the nucleotide sequence of one strand of a first double-stranded nicking agent recognition sequence (NARS) and is at least substantially complementary to, and optionally is exactly complementary to, a trigger oligonucleotide primer (trigger ODNP). (B) Providing the trigger ODNP, and hybridizing the trigger ODNP to T1. (C) Extending the trigger ODNP to form a hybrid (H1) comprising extended trigger ODNP hybridized to T1, where H1 comprises the first double-stranded NARS. (D) Nicking H1 at a nicking site with a nicking agent (NA) that recognizes the NARS, to thereby provide a fragment having a 5′ end at the nicking site, where the fragment is named A1. (E) Providing a second template nucleic acid molecule (T2) that is at least substantially complementary to, and optionally is exactly complementary to, A1. (F) Hybridizing A1 to T2. (G) Extending A1 to form a hybrid (H2) comprising extended A1 hybridized to T2, where H2 comprises a second NARS. (H) Nicking H2 with a second NA that recognizes the second NARS so as to provide a fragment, where the fragment has a 5′ terminus at the nicking site, and the fragment is named A2. (I) Extending the 3′ terminus at the nicking site in H2 to re-form H2. (J) Repeating steps (H) and (I) to thereby amplify A2.

In a related aspect, the present invention provides a method for amplifying a nucleic acid molecule, where the method includes: (A) Forming a mixture that includes (i), (ii) and (iii), where (i) is a first single-stranded nucleic acid molecule having a nucleotide sequence (S1), (ii) is a second single-stranded nucleic acid molecule having the nucleotide sequence of the antisense strand of a nicking agent recognition sequence (NARS), wherein a nucleotide sequence substantially complementary, or optionally exactly complementary, to S1 is present both 3′ and 5′ to the nucleotide sequence of the antisense strand of the NARS, and (iii) is a nicking agent (NA) that recognizes the NARS, a DNA polymerase, and one or more deoxynucleoside triphosphate(s). (B) Maintaining the mixture (A) under conditions that amplify a single-stranded nucleic acid molecule using single-stranded nucleic acid molecule (A)(ii) as a template. Optionally, the amplified nucleic acid molecule has a sequence that is exactly identical to S1.

In another aspect, the present invention provides a tandem nucleic acid amplification system, where this system includes: A first primer extension means for amplifying a first single-stranded nucleic acid (A1), and also includes a second primer extension means for amplifying a second single-stranded nucleic acid (A2); where A1 is the primer for the second primer extension means for amplifying A2, and both the first and second primer extension means are contained within a single reaction vessel and require the presence of a nicking agent (NA), in order to amplify a nucleic acid molecule. In optional embodiments of this system: the NA for the first primer extension means is identical to the NA for the second primer extension means; the first means for amplifying A1 includes combining a first oligonucleotide primer (trigger ODNP), a first template nucleic acid molecule (T1) that is at least substantially complementary to, and optionally is exactly complementary to the trigger ODNP, a first nicking agent (NA), and a first DNA polymerase, wherein the extension of the trigger ODNP using T1 as a template produces a first nicking agent recognition sequence (NARS) that is recognizable by the first NA. Also optionally, the second means for amplifying A2 includes the nucleic acid (A1), a second template nucleic acid (T2) at least substantially complementary to, and optionally is exactly complementary to A1, a second NA, and the DNA polymerase, wherein the extension of A1 using T2 as a template produces a second NARS that is recognizable by the second NA. Optionally, the first polymerase is identical to the second polymerase.

In another aspect, the present invention provides a method for exponential amplification of a nucleic acid molecule (A2), where the method includes: (A) Amplifying a nucleic acid molecule (A1) using a first template nucleic acid (T1) that includes the nucleotide sequence of one strand of a first nicking agent recognition sequence (NARS). The amplification is performed in the presence of a first nicking endonuclease (NA) that recognizes the first NARS, and a first DNA polymerase. (B) Amplifying A2 using a second template nucleic acid (T2) that includes the nucleotide sequence of one strand of a second NARS as a template and A1 as a primer, in the presence of a second NA and a second DNA polymerase, where the first and second polymerases are optionally the same polymerase.**

The present invention also provides methods for determining the presence or absence of a target nucleic acid in a sample. These methods are particularly useful in diagnosis. In one aspect, the present invention provides a method for determining the presence or the absence of a target nucleic acid in a sample, where this method includes: (A) Forming a mixture that includes: (i) the nucleic acid molecules of the sample, (ii) a first single-stranded nucleic acid molecule (T1) comprising from 3′ to 5′: (a) a first nucleotide sequence that is at least substantially complementary, and optionally is exactly complementary, to the target nucleic acid molecule, (b) the nucleotide sequence of the antisense strand of a first nicking agent recognition sequence (NARS), and (c) a second nucleotide sequence, (iii) a second single-stranded nucleic acid molecule (T2) comprising from 3′ to 5′: (a) a first nucleotide sequence that is at least substantially identical, and optionally is exactly identical to, (A)(ii)(c), i.e., the second nucleotide sequence of T1, (b) the nucleotide sequence of the antisense strand of a second NARS, and (c) a second sequence, (iv) a first nicking endonuclease (NA) that recognizes the first NARS, a second NA that recognizes the second NARS, a DNA polymerase, and one or more deoxynucleoside triphosphate(s). (B) Maintaining the mixture (A) at conditions that amplify a single-stranded nucleic acid molecule (A2) using the second sequence of T2 as a template if the target nucleic acid is present in the sample. (C) Detecting the presence or the absence of A2 to determine the presence, or the absence, of the target nucleic acid in the sample. In a related aspect, the present invention provides a method for determining the presence or the absence of a target nucleic acid in a sample, where the method includes: (A) Forming a mixture that includes: (i) the nucleic acid molecules of the sample; (ii) a first single-stranded nucleic acid molecule (T1) comprising, from 3′ to 5′: (a) a nucleotide sequence that is at least substantially complementary, and optionally is exactly complementary, to the target nucleic acid molecule, and (b) the nucleotide sequence of the sense strand of a first nicking agent recognition sequence (NARS), (iii) a second single-stranded nucleic acid molecule (T2) comprising, from 3′ to 5′: (a) a nucleotide sequence that is at least substantially complementary, and optionally is exactly complementary, to the nucleotide of T1 that is located 3′ to the nucleotide of the sense strand of the first NERS, and (b) the nucleotide sequence of the antisense strand of a second NARS, and (iv) a first nicking endonuclease (NA) that recognizes the first NARS, a second NA that recognizes the second NARS, a DNA polymerase, and one or more deoxynucleoside triphosphate(s). (B) Maintaining the mixture (A) at conditions that amplify a single-stranded nucleic acid molecule (A2) using T2 as a template if the target nucleic acid is present in the sample. (C) Detecting the presence or the absence of A2 to determine the presence, or the absence, of the target nucleic acid in the sample. In a related aspect, the present invention provides a method for determining the presence or absence of a target nucleic acid molecule, where the target nucleic acid includes a first nicking endonuclease recognition sequence (NERS), the method including: (A) Forming a mixture comprising: (i) the nucleic acid molecules of the sample, (ii) a single-stranded nucleic acid molecule (T2) comprising from 3′ to 5′: (a) a sequence that is at least substantially identical, and optionally is exactly identical, to a portion of the target nucleic acid molecule located 5′ to the nucleotide of the antisense strand of the first NERS, and (b) the nucleotide sequence of the antisense strand of a second NERS, and (iii) a first nicking endonuclease (NE) that recognizes the first NERS; a second NE that recognizes the second NERS, a DNA polymerase, and one or more deoxynucleoside triphosphate(s). (B) Maintaining mixture (A) at conditions that amplify a single-stranded nucleic acid molecule (A2) using T2 as a template if the target nucleic acid is present in the sample. (C) Detecting the presence or absence of A2 to determine the presence or absence of the target nucleic acid in the sample. In a related aspect, the present invention provides a method for determining the presence or absence of a target nucleic acid that comprises a first nicking endonuclease recognition sequence (NERS) in a sample, where the method includes: (A) Forming a mixture comprising: (i) the nucleic acid molecules of the sample, (ii) a first single-stranded nucleic acid molecule (T1) that is substantially identical, and optionally is exactly identical, to one strand of the target nucleic acid and comprise a sequence of the antisense strand of the first NERS, (iii) a second single-stranded nucleic acid molecule (T2) comprising from 3′ to 5′: (a) a nucleotide sequence that is at least substantially identical, and optionally is exactly identical, to a portion of T1 located 5′ to the nucleotide of the antisense strand of the first NERS, and (b) the nucleotide sequence of the antisense strand of a second NERS, and (iv) a first nicking endonuclease (NE) that recognizes the first NERS, a second NE that recognizes the second NERS, a DNA polymerase, and one or more deoxynucleoside triphosphate(s). (B) Maintaining the mixture (A) at conditions that amplify a single-stranded nucleic acid molecule (A2) using T2 as a template if the target nucleic acid is present in the sample. (C) Detecting the presence or absence of A2 to determine the presence or absence of the target nucleic acid in the sample. In a related aspect, the present invention provides a method for determining the presence or absence of a target nucleic acid in a sample, where the method includes: (A) Forming a mixture of a first oligonucleotide primer (ODNP), a second ODNP, and the nucleic acid molecules of the sample, wherein if (i) the target nucleic acid is a double-stranded nucleic acid having a first strand and a second strand, then the first ODNP comprises the nucleotide sequence of the sense strand of a first restriction endonuclease recognition sequence (RERS) and a nucleotide sequence that is at least substantially complementary, optionally exactly complementary, to a first portion of the first strand of the target nucleic acid, and the second ODNP comprises a nucleotide sequence that is at least substantially complementary, optionally exactly complementary, to a second portion of the second strand of the target nucleic acid and comprises the nucleotide sequence of the sense strand of a second RERS, the second portion being located 3′ to the complement of the first portion in the second strand of the target nucleic acid. However, if (ii), the target nucleic acid is a single-stranded nucleic acid, then the first ODNP comprises a nucleotide sequence of a sense strand of a first RERS and a nucleotide sequence that is at least substantially identical to, and optionally is exactly identical to, a first portion of the target nucleic acid, and the second ODNP comprises a nucleotide sequence that is at least substantially complementary to, and optionally is exactly complementary to, a second portion of the target nucleic acid and comprises a sequence of the sense strand of a second RERS, the second portion being located 5′ to the first portion in the target nucleic acid. (B) Subjecting the mixture to conditions that, if the target nucleic acid is present in the sample, (i) extends the first and the second ODNPs to produce an extension product comprising both the first and the second RERSs; (ii) amplifies a first single-stranded nucleic acid fragment (A1) using one strand of the extension product of step (B)(i) as a template in the presence of one or more restriction endonucleases (REs) that recognize the first and the second RERSs; (iii) in the presence of a second single-stranded nucleic acid molecule (T2) capable of annealing to A1, amplifies a third single-stranded nucleic acid fragment (A2) using A1 as a template, wherein A1, A2 or both preferably have at most 25 nucleotides, and wherein T2 comprises, from 5′ to 3′: (a) the nucleotide sequence of the antisense strand of a third RERS, and (b) a sequence that is at least substantially complementary, and optionally exactly complementary, to A1. (C) Detecting the presence or absence of A2 to determine the presence or absence of the target nucleic acid in the sample. In a related aspect, the present invention provides a method for determining the presence or absence of a target nucleic acid in a sample, where the method includes: (A) Forming a mixture of a first oligonucleotide primer (ODNP), a second ODNP, and the nucleic acid molecule of the sample, wherein if (i) the target nucleic acid is a double-stranded nucleic acid having a first strand and a second strand, then the first ODNP comprises a nucleotide sequence of a sense strand of a first nicking endonuclease recognition sequence (NERS) and a nucleotide sequence that is at least substantially complementary to, and optionally is exactly complementary to, a first portion of the first strand of the target nucleic acid, and the second ODNP comprises a nucleotide sequence that is at least substantially complementary to, and optionally is exactly complementary to, a second portion of the second strand of the target nucleic acid and comprises a sequence of the sense strand of a second NERS, the second portion being located 3′ to the complement of the first portion in the second strand of the target nucleic acid, however, if (ii) the target nucleic acid is a single-stranded nucleic acid, then the first ODNP comprises a nucleotide sequence of a sense strand of a first NERS and a nucleotide sequence that is at least substantially identical to, and optionally is exactly identical to, a first portion of the target nucleic acid, and the second ODNP comprises a nucleotide sequence that is at least substantially complementary to, and optionally is exactly complementary to, a second portion of the target nucleic acid and comprises a sequence of the sense strand of a second NERS, the second portion being located 5′ to the first portion in the target nucleic acid. (B) Subjecting the mixture to conditions that, if the target nucleic acid is present in the sample, (i) extend the first and the second ODNPs to produce an extension product comprising both the first and the second NERSs; (ii) amplify a first single-stranded nucleic acid fragment (A1) using one strand of the extension product of step (B)(i) as a template in the presence of one or more nicking endonucleases (NEs) that recognize the first and the second NERSs; (iii) in the presence of a second single-stranded nucleic acid molecule (T2) capable of annealing to A1, amplify a third single-stranded nucleic acid fragment (A2) using A1 as a template, wherein A1, A2 or both optionally have at most 25 nucleotides, and wherein T2 comprises, from 5′ to 3′: (a) a sequence of the antisense strand of a third NERS, and (b) a sequence that is at least substantially complementary to, and optionally is exactly complementary to, A1. (C) Detecting the presence or absence of A2 to determine the presence or absence of the target nucleic acid in the sample. In a related aspect, the present invention provides a method for determining the presence or absence of a target nucleic acid in a sample, where the method includes: (A) Forming a mixture comprising: (i) the nucleic acid molecules of the sample, (ii) a single-stranded nucleic acid probe that comprises, from 3′ to 5′, a sequence that is at least substantially complementary to, and optionally is exactly complementary to, the 5′ portion of the target nucleic acid, and a sequence of the antisense strand of a first nicking agent recognition sequence (NARS). (B) Separating hybridized from unhybridized probe as formed in step (A). (C) Performing an amplification reaction with the hybridized probe in the presence of a first nicking agent (NA) that recognizes the first NARS. (D) Providing a single-stranded nucleic acid molecule (T2) comprising, from 5′ to 3′: (i) a sequence of the antisense strand of a second NARS, and (ii) a sequence that is at least substantially identical to, and optionally is exactly identical to, the portion of the first single-stranded nucleic acid probe located 5′ to the nucleotide of the antisense strand of the first NARS. (E) Performing an amplification reaction in the presence of a second NA that recognizes the second NARS. (F) Detecting the presence or absence of the amplification product of step (E) to determine the presence or absence of the target nucleic acid in the sample. In a related aspect, the present invention provides a method for determining the presence or absence of a target nucleic acid in a sample, where the method includes: (A) Forming a mixture comprising: (i) the nucleic acid molecules of the sample, (ii) a single-stranded nucleic acid probe that comprises, from 5′ to 3′: (a) a nucleotide sequence that is at least substantially complementary, and optionally is completely complementary, to the 3′ portion of the target nucleic acid, and (b) a nucleotide sequence of the antisense strand of a first NARS. (B) Separating hybridized probe from unhybridized probe as formed in step (A); (C) Performing an amplification reaction in the presence of hybridized probe and a first nicking agent (NA) that recognizes the first NARS. (D) Providing a single-stranded nucleic acid molecule (T2) comprising, from 5′ to 3′: (i) a sequence of the antisense strand of a second NARS, and (ii) a sequence that is at least substantially complementary to, and optionally is exactly complementary to, the portion of the first single-stranded nucleic acid probe located 5′ to the nucleotide of the antisense strand of the first NARS. (E) Performing an amplification reaction in the presence of a second NA that recognizes the second NARS. (F) Detecting the presence or absence of the amplification product of step (E) to determine the presence or absence of the target nucleic acid in the sample. In a related aspect, the present invention provides a method for determining the presence or absence of a target nucleic acid in a sample, where the method includes: (A) Forming a mixture comprising: (i) the nucleic acid molecules of the sample, (ii) a partially double-stranded nucleic acid probe that comprises: (a) the nucleotide sequence of the sense strand of a first NARS, the nucleotide sequence of the antisense strand of the first NARS, or both; and (b) a 5′ overhang in the strand that the strand itself or an extension product thereof contains a nicking site (NS) nickable by a first nicking agent (NA) that recognizes the first NARS, or a 3′ overhang in the strand that neither the strand nor an extension product thereof contains the NS, wherein an overhang comprises a nucleotide sequence that is at least substantially complementary to, and optionally is exactly complementary to, the target nucleic acid. (B) Separating hybridized probe from unhybridized probe as formed in the mixture of step (A). (C) Performing an amplification reaction in the presence of hybridized probe and a first nicking agent (NA) that recognizes the first NARS. (D) Providing a single-stranded nucleic acid molecule (T2) comprising, from 5′ to 3′ (i.e., in going toward the 3′ end of the molecule, the following components are sequentially present in T2): (i) a nucleotide sequence of the antisense strand of a second NARS, and (ii) a nucleotide sequence that is at least substantially identical to, and optionally is exactly identical to, the portion of the nucleic acid probe located 5′ to the nucleotide of the antisense strand of the first NARS. (E) Performing an amplification reaction in the presence of a second NA that recognizes the second NARS. (F) Detecting the presence or absence of the amplification product of step (E) to determine the presence or absence of the target nucleic acid in the sample.

In other aspects, the present invention provides the following methods. These methods are useful, e.g., in determining the presence or absence of a genetic variation at a defined location in a single-stranded target nucleic acid. In one aspect, a method of the present invention includes: (A) Providing a single-stranded nucleic acid (A1) that includes a nucleotide sequence that is exactly complementary to a portion of a target nucleic acid, where this portion of the target nucleic acid includes a nucleotide or nucleotides at the defined location. A1 is provided via an amplification process, whereby multiple copies of A1 are prepared in the presence of a first nicking agent. (B) Performing an amplification reaction in the presence of (i), (ii), (iii) and (iv), where (i) is a single-stranded template nucleic acid (T2) that comprises, from 3′ to 5′: (a) a first nucleotide sequence that is at least substantially complementary, and preferably is exactly complementary, to A1, and also includes the genetic variation, (b) the nucleotide sequence of the antisense strand of a nicking agent recognition sequence (NARS) that is recognizable by a second nicking agent, (c) a second sequence, (ii) is the second nicking agent, (iii) is a DNA polymerase, and (iv) is one or more deoxynucleoside triphosphates. This amplification reaction is performed under conditions that amplify a single-stranded nucleic acid molecule (A2) using at least a portion of the second sequence of the T2 molecule. However, this amplification reaction only occurs if A1 includes the complementary nucleotide(s) of the genetic variation. (C) Detecting the presence or absence of A2. This detection step allow for the determination of the presence or absence of the genetic variation at the defined location of the target nucleic acid. Optionally, A1 is provided by (a) forming a mixture of a first ODNP, a second ODNP, and the target nucleic acid, wherein (i) the first ODNP includes a nucleotide sequence of one strand of a first RERS and a nucleotide sequence that is at least substantially identical to, and optionally is exactly identical to, a nucleotide sequence of the target nucleic acid located 5′ to the complement of the genetic variation, and (ii) the second ODNP includes a nucleotide sequence of one strand of a second RERS and a nucleotide sequence that is at least substantially complementary to, and optionally is exactly complementary to, a nucleotide sequence of the target nucleic acid located 3′ to the genetic variation; (b) extending the first and the second ODNPs in the presence of deoxyribonucleoside triphosphates and at least one modified deoxyribonucleoside triphosphate to produce an extension product comprising both the first and the second RERSs; and (c) amplifying the single-stranded nucleic acid fragment A1 using one strand of the extension product of step (b) as a template in the presence of restriction endonucleases (REs) that recognize the first RERS and the second RERS, where, in a further optional embodiment, the first, second and third RERSs are identical to each other. Alternatively, A1 may be provided by (a) forming a mixture of a first oligonucleotide primer (ODNP), a second ODNP and the target nucleic acid, wherein (i) the first ODNP includes a nucleotide sequence that is at least substantially identical to, and optionally is exactly identical to, a nucleotide sequence of the target nucleic acid located 5′ to the genetic variation, and (ii) the second ODNP includes a nucleotide sequence that is at least substantially complementary, and optionally is exactly complementary, to a nucleotide sequence of the target nucleic acid located 3′ to the genetic variation, where the first and the second ODNPs each further comprise a nucleotide sequence of the sense strand of a nicking endonuclease recognition sequence (NERS); (b) extending the first and the second ODNPs to produce an extension product comprising two NERSs; and (c) amplifying the single-stranded nucleic acid fragment A1 using one strand of the extension product of step (b) as a template in the presence of one or more nicking endonucleases (NEs) that recognizes the NERS(s), where optionally the NERSs in the first ODNP, the second ODNP and T2 are identical to each other. In a related aspect, the present invention provides a method for identifying a genetic variation at a defined location in a single-stranded target nucleic acid, where the method includes: (A) Providing a single-stranded nucleic acid molecule (A1) that includes a sequence that is exactly complementary to a portion of the target nucleic acid, where the portion of the target nucleic acid comprises a genetic variation at a defined location. A1 is provided by an amplification process that is performed in the presence of a first nicking agent. (B) Performing an amplification reaction in the presence of (i), (ii), (iii) and (iv), where (i) is multiple single-stranded template nucleic acids (T2), each T2 comprises, as viewed from the 3′ to the 5′ direction of T2: (a) a first sequence that is at least substantially complementary, and optionally is exactly complementary, to A1, where this first sequence also includes one of the potential genetic variations at the defined position of the target nucleic acid, (b) the nucleotide sequence of the antisense strand of a NARS that is recognizable by a second nicking agent, and (c) a second sequence that uniquely correlates to the potential genetic variation, wherein the multiple T2 molecules, in combination, comprise all the potential genetic variations at the defined position of the target nucleic acid, (ii) the second nicking agent, (iii) a DNA polymerase, and (iv) one or more deoxynucleoside triphosphates. These components (i)-(iv) are amplified under conditions that selectively amplify a single-stranded nucleic acid molecule (A2) using at least a portion of the second sequence of a T2 molecule as a template, where T2 molecule includes the genetic variation of the target nucleic acid. When a sequence “uniquely correlates” to a potential genetic variation, then detection of that sequence effectively indicates whether the genetic variation is present, and if present, in what form. (C) Characterizing the A2 amplified in step (B) to identify the gene variation of the target nucleic acid. Optionally in this related method, one of more of the following criteria may be applied in describing the method: the second sequence of each of the T2 molecules is at least substantially identical, and optionally is exactly identical, to the first sequence of the same T2 molecule; the second nicking agent nicks 5′ to the nucleotide of the sense strand of the nicking agent recognition sequence; the portion of the second sequence of each T2 molecule located immediately 5′ to the nicking site nickable by the second nicking agent is exactly identical to the first sequence of the same T2 molecule. In methods of the present invention directed to detecting a genetic variation, the following criteria may additionally be used to describe a method, where any two or more of the following criteria may be combined in describing the method, and where the following criteria are exemplary only in that other criteria may be provided elsewhere herein, where these other criteria include the criteria provide above in connection with other methods of the present invention: the genetic variation is a single nucleotide polymorphism; the genetic variation is associated with a disease; the genetic variation is associated with a human genetic disease; the genetic variation is associated with drug resistance of a pathogenic microorganism; the nicking agent is N.BstNB I; amplification, e.g., step (B) as described above, is performed under isothermal conditions, e.g., at 50° C.-70° C.; the DNA polymerase is selected from exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase, and T4 DNA polymerase homoenzyme, or any combination thereof, e.g., exo⁻ Vent, exo⁻ Deep Vent, exo⁻ Bst, exo⁻ Bca, or 9° Nm™ DNA polymerase; detection, e.g., step (C) as described above, is performed at least partially by the use of a technique selected from the group consisting of mass spectrometry, liquid chromatography, fluorescence polarization, and electrophoresis, or any combination thereof, e.g., step (C) is performed at least partially by both liquid chromatography and mass spectrometry; the first ODNP is immobilized; the second ODNP is immobilized; both the first and second ODNPs are immobilized; the target nucleic acid is immobilized; T2, or each T2, is immobilized; immobilization is to a solid support via covalent attachment; the second sequence of the T2 is at least substantially identical to, and optionally is exactly identical to, the first sequence and comprises the genetic variation; the second sequence of the T2 is exactly identical to the first sequence and comprises the genetic variation; the second nicking agent nicks 5′ to the nucleotide of the sense strand of the nicking agent recognition sequence; the portion of the second sequence of the T2 located immediately 5′ to the nicking site nickable by the second nicking agent is exactly identical to the first sequence of the T2 molecule.

In other aspects, the present invention provides additional methods. These methods may be used, e.g., to determine the presence or absence of a junction between an upstream exon (Exon A) and a downstream exon (Exon B) in a cDNA molecule. The cDNA molecule may be single-stranded or double-stranded. The double-stranded cDNA molecule will have a strand that includes the sense strand of Exon A and the sense strand of Exon B, where the sense strand of Exon A has the same nucleotide sequence that is found in the corresponding mRNA molecule that encodes Exon A, but for the change of U (in the mRNA molecule) for T (in the DNA). A single-stranded cDNA molecule can be either strand of the double-stranded cDNA molecule as just described. In one aspect, the present invention provides a method that includes: (A) Providing a nucleic acid molecule (N1) that is at least partially double-stranded, and in one embodiment is completely double-stranded, where N1 includes features (i) and (ii), where (i) is either or both of a) and b), where a) is the nucleotide sequence of the sense strand of a first nicking agent recognition sequence (NARS) and b) is the nucleotide sequence of the antisense strand of the first NARS, and (ii) is at least one strand of a portion of the cDNA molecule if the cDNA molecule is double-stranded, or a portion of the cDNA if the cDNA molecule is single-stranded, where the portion of the cDNA molecule is suspected to contain the junction between Exon A and Exon B. (B) Amplifying a first single-stranded nucleic acid molecule (A1) in the presence of a nicking agent (NA) that recognizes the first NARS, a DNA polymerase, and one or more deoxynucleoside triphosphate(s). As a template for the polymerase, the amplifying uses either the portion of the single-stranded cDNA or one strand of the portion of the double-stranded cDNA. (C) Providing a second single-stranded nucleic acid molecule (T2) that includes, from 5′ to 3′: (i) a first nucleotide sequence comprising (a) or (b), where (a) is a 3′ portion of the sense strand of Exon A, which is linked at its 3′ terminus to a 5′ portion of the sense strand of Exon B, where the 3′ portion is linked to the 5′ terminus of the 5′ portion, and (b) is a 5′ portion of the antisense strand of Exon A, which is linked at its 5′ terminus to a 3′ portion of the antisense strand of Exon B, where the 5′ portion is linked to the 3′ terminus of the 3′ portion. If the cDNA contains the junction between Exon A and Exon B, then the first nucleotide sequence of the T2 is at least substantially complementary to, and optionally is exactly complementary to, the A1 molecule, but if the cDNA does not contain the junction between Exon A and Exon B, then the first nucleotide sequence of T2 is not substantially complementary to the A1 molecule, (ii) a sequence of the antisense strand of a second NARS, and (iii) a second sequence. (D) If the junction between Exon A and Exon B is present in the target cDNA molecule, then the method performs an amplification reaction that amplifies a third single-stranded nucleic acid molecule (A2) using at least a portion of the second sequence of T2 as a template. (E) Detecting the presence or absence of the A2 to determine the presence or absence of the junction in the cDNA molecule. In a related aspect, the present invention provides a method for determining the presence or absence of a junction between an upstream exon (Exon A) and a downstream exon (Exon B) of a gene in a cDNA molecule, where the method includes: (A) Forming a mixture of a first oligonucleotide primer (ODNP), a second ODNP, and the cDNA molecule, wherein (i) the first ODNP comprises a nucleotide sequence that is at least substantially complementary, and optionally is exactly complementary, to a portion of the antisense strand of Exon A near the 5′ terminus of Exon A in the antisense strand, (ii) the second ODNP includes a nucleotide sequence that is at least substantially complementary to, and optionally is exactly complementary to, a portion of the sense strand of Exon B near the 5′ terminus of Exon B in the sense strand, and (iii) at least one of the first ODNP and the second ODNP further comprises the nucleotide sequence of the sense strand of a first nicking agent recognition sequence (NARS). (B) Performing a first amplification reaction in the presence of a nicking agent (NA) that recognizes the first NARS under the conditions that amplify a first single-stranded nucleic acid (A1) if both Exon A and Exon B are present in the cDNA. (C) Providing a second single-stranded nucleic acid molecule (T2) comprising, from 5′ to 3′: (i) a first sequence comprising (a) a 3′ portion of the sense strand of Exon A linked at the 3′ terminus of the 3′ portion to a 5′ portion of the sense strand of Exon B at the 5′ terminus of the 5′ terminus, or (b) a 5′ portion of the antisense strand of Exon A linked at the 5′ terminus of the 5′ portion to a 3′ portion of the antisense strand of Exon B at the 3′ terminus of the 3′ portion, wherein if the cDNA contains the junction between Exon A and Exon B, the first sequence of T2 is at least substantially complementary to, and optionally is exactly complementary to, the A1 molecule, but if the cDNA does not contain the junction between Exon A and Exon B, then T2 is not substantially complementary to the A1 molecule, (ii) a sequence of the antisense strand of a second NARS, and (iii) a second sequence. (D) Performing an amplification reaction that amplifies a third single-stranded nucleic acid molecule (A2) using at least a portion of the second sequence of T2 as a template if the junction between Exon A and Exon B is present in the target cDNA molecule. (E) Detecting the presence or absence of the A2 to determine the presence or absence of the junction in the cDNA molecule. In a related aspect, the present invention provides a method for determining the presence or absence of a junction between an upstream exon (Exon A) and a downstream exon (Exon B) of a gene in a cDNA molecule, where the method includes: (A) Forming a mixture of a first oligonucleotide primer (ODNP), a second ODNP, and the cDNA molecule, wherein (i) the first ODNP comprises (a) a nucleotide sequence that is at least substantially complementary to, and optionally is exactly complementary to, a portion of the antisense strand of Exon A near the 5′ terminus of Exon A in the antisense strand, and (b) a nucleotide sequence of the sense strand of a first nicking agent recognition sequence (NARS); and (ii) the second ODNP comprises (a) a nucleotide sequence that is at least substantially complementary to, and optionally is exactly complementary to, a portion of the sense strand of Exon B near the 5′ terminus of Exon B in the sense strand, and (b) a sequence of the sense strand of a second NARS. (B) Performing a first amplification reaction in the presence of a first nicking agent (NA) that recognizes the first NARS and a second NA that recognizes the second NARS under the conditions that amplify a first single-stranded nucleic acid (A1) if both Exon A and Exon B are present in the cDNA. (C) Providing a second single-stranded nucleic acid molecule (T2) comprising, from 5′ to 3′: (i) a first sequence comprising (a) a 3′ portion of the sense strand of Exon A linked at the 3′ terminus of the 3′ portion to a 5′ portion of the sense strand of Exon B at the 5′ terminus of the 5′ terminus, or (b) a 5′ portion of the antisense strand of Exon A linked at the 5′ terminus of the 5′ portion to a 3′ portion of the antisense strand of Exon B at the 3′ terminus of the 3′ portion, wherein if the cDNA contains the junction between Exon A and Exon B, the first sequence of the T2 is at least substantially complementary to, and optionally is exactly complementary to, the A1 molecule, but if the cDNA does not contain the junction between Exon A and Exon B, the T2 is not substantially complementary to the A1 molecule, (ii) a sequence of the antisense strand of a second NARS, and (iii) a second sequence. (D) Performing an amplification reaction that amplifies a third single-stranded nucleic acid molecule (A2) using at least a portion of the second sequence of T2 as a template if the junction between Exon A and Exon B is present in the target cDNA molecule. (E) Detecting the presence or absence of the A2 to determine the presence or absence of the junction in the cDNA molecule. Optionally, the first, second and third NARS are identical. In additional optional embodiments, one or more of the following criteria may be used to describe the method, where these criteria are exemplary only in that other criteria as set forth herein in connection with methods of the invention may also be used to further describe this method: N1 comprises the nucleotide sequence of the antisense strand of the first NARS; N1 comprises the nucleotide sequence of the sense strand of the first NARS; both the first and the second NAs are restriction endonucleases (REs), and at least one of the nucleoside triphosphate(s) is modified; A1 is from 8 to 24 nucleotides in length; A1 is from 12 to 17 nucleotides in length; A2 is from 8 to 24 nucleotides in length; A2 is from 12 to 17 nucleotides in length; the DNA polymerase is 5′→3′ exonuclease deficient; the DNA polymerase has a strand displacement activity; each of steps (B) and (D) is performed in the presence of a strand displacement facilitator; N1 is immobilized; T2 is immobilized; the cDNA is immobilized; the first ODNP is immobilized; the second ODNP is immobilized; both the first and second ODNPs are immobilized.

The following criteria may be used, alone or in any combination, to further describe the methods of the present invention as outlined above and elsewhere herein, where these criteria are exemplary only and other criteria may be set forth elsewhere herein: the first NARS is identical to the second NARS; the first nicking agent is the same as the second nicking agent; any one or more NARSs in a method is a NERS; both the first and the second NAs are a nicking endonuclease (NE); the NE is N.BstNB I; the NE is N.AIw I; both the first and the second NEs are N.BstNB I; at least one of the first or second nicking agents is a nicking endonuclease; both the first and the second NAs are restriction endonucleases (REs); the first, second and third NARSs (when three NARSs are specified in an embodiment of the invention) are identical to each other; each of the first, second and third NARSs is recognized by a nicking endonuclease; at least one of a first, second and third NARS is recognized by a nicking endonuclease; any one, or any two, or any three, or any four, or any five, or any six, or any seven, or any eight, or any nine, or any ten etc. steps of the method (e.g., steps (A), (B), (C) and (D), or e.g., steps (a) through (j)) are performed in a single vessel; the amplification of a single-stranded nucleic acid fragment is performed under isothermal conditions; each amplification reaction is performed at one or more temperatures within the range of 50° C.-70° C.; each amplification reaction is performed at, or at about, 60° C.; each amplification reaction is performed at temperatures between a highest temperature and a lowest temperature, where the highest temperature is within 20° C. of the lowest temperature; each amplification reaction is performed at temperatures between a highest temperature and a lowest temperature, where the highest temperature is within 15° C. of the lowest temperature; each amplification reaction is performed at temperatures between a highest temperature and a lowest temperature, where the highest temperature is within 10° C. of the lowest temperature; each amplification reaction is performed at temperatures between a highest temperature and a lowest temperature, where the highest temperature is within 5° C. of the lowest temperature; N1 includes the nucleotide sequence of the sense strand of the first NERS; N1 includes the nucleotide sequence of the antisense strand of the first NERS; both the first and the second NAs are restriction endonucleases (REs); N1 is provided by annealing a trigger oligonucleotide primer (ODNP) and a single-stranded nucleic acid (T1), where T1 includes the nucleotide sequence of either the sense strand or the antisense strand of the first NERS; when N1 is provided by annealing a trigger oligonucleotide primer (ODNP) to a single-stranded target nucleic acid (T1) that comprises, from 5′ to 3′: (A) a sequence of an antisense strand of the first NARS; and (B) a sequence that is at least substantially complementary to, and optionally is exactly complementary to, at least a portion of the trigger ODNP, then the nucleotide (B) of T1 is exactly complementary to at least a portion of the trigger ODNP; T1 is substantially identical to T2; the 3′ terminus of T2 is linked to a phosphate group; the 3′ terminus of T1 is linked to a phosphate group; T1 is exactly identical to T2; T1 is neither substantially nor exactly identical to T2; the nucleotide sequence of T2 that is at least substantially identical to, and optionally is exactly identical to, a portion of N1 located 5′ to the antisense strand of the NARS in N1 is, in fact, exactly identical to a portion of N1 located 5′ to the antisense strand of the first NARS; when T3 includes a sequence that is at least substantially identical to, and optionally is exactly identical to, at least a portion of the template nucleotide sequence of T2, then in one embodiment T3 includes a sequence that is exactly identical to at least a portion of the template nucleotide sequence T2; A2 includes a nucleotide sequence that is at least substantially identical to, and optionally is exactly identical to, a nucleotide sequence in A1; A2 includes a nucleotide sequence that is exactly identical to a nucleotide sequence in A1; A1 includes a nucleotide sequence that is at least substantially identical to, and optionally is exactly identical to, a nucleotide sequence in A2; A1 includes a nucleotide sequence that is exactly identical to a nucleotide sequence in A2; A2 and A1 are identical; A1 is substantially identical to A2; A2 is substantially identical to A1; A1 is exactly identical to A2; A1 is neither substantially nor exactly identical to A2; A1 is substantially identical to the trigger ODNP; A1 is exactly identical to the trigger ODNP; A2 is substantially identical to the trigger ODNP; A2 is exactly identical to the trigger ODNP; A1 is at least 5, or at least 6, or at least 7, or at least 8, or at least 9, or at least 10, or at least 11, or at least 12, or at least 13, or at least 14, or at least 15, or at least 16, or at least 17, or at least 18, or at least 19, or at least 20, or at least 21, or at least 22, or at least 23, or at least 24, or at least 25 nucleotides in length, while additionally, or alternatively, A1 is no more than 40, or no more than 39, or no more than 38, or no more than 37, or no more than 36, or no more than 35, or no more than 34, or no more than 33, or no more than 32, or no more than 31, or no more than 30, or no more than 29, or no more than 28, or no more than 27, or no more than 26, or no more than 25, or no more than 24, or no more than 23, or no more than 22, or no more than 21, or no more than 20, or no more than 19, or no more than 18, or no more than 17, or no more than 16, or no more than 15, or no more than 14, or no more than 13, or no more than 12, or no more than 11, or no more than 10 nucleotides in length, where any stated upper limit on the nucleotide length of A1 may be combined with any stated lower limit on the nucleotide length of A1, so that A1 may be, for example, from 8 to 24 nucleotides in length, or from 12 to 17 nucleotides in length; A2 is at least 5, or at least 6, or at least 7, or at least 8, or at least 9, or at least 10, or at least 11, or at least 12, or at least 13, or at least 14, or at least 15, or at least 16, or at least 17, or at least 18, or at least 19, or at least 20, or at least 21, or at least 22, or at least 23, or at least 24, or at least 25 nucleotides in length, while additionally, or alternatively, A2 is no more than 40, or no more than 39, or no more than 38, or no more than 37, or no more than 36, or no more than 35, or no more than 34, or no more than 33, or no more than 32, or no more than 31, or no more than 30, or no more than 29, or no more than 28, or no more than 27, or no more than 26, or no more than 25, or no more than 24, or no more than 23, or no more than 22, or no more than 21, or no more than 20, or no more than 19, or no more than 18, or no more than 17, or no more than 16, or no more than 15, or no more than 14, or no more than 13, or no more than 12, or no more than 11, or no more than 10 nucleotides in length, where any stated upper limit on the nucleotide length of A2 may be combined with any stated lower limit on the nucleotide length of A2, so that A2 may be, for example, from 8 to 24 nucleotides in length, or from 12 to 17 nucleotides in length; the initial number of T2 molecules is more than the initial number of T1 molecules; N1 is derived from a genomic DNA; N1 is a portion of a genomic DNA; the target nucleic acid is one strand of a denatured double-stranded nucleic acid; the target nucleic acid is one strand of double-stranded genomic nucleic acid or cDNA; the target nucleic acid is an RNA molecule; the target nucleic acid is derived from nucleic acid obtained from a bacterium; the target nucleic acid is derived from nucleic acid obtained from a virus; the target nucleic acid is derived from nucleic acid obtained from a fungus; the target nucleic acid is derived from nucleic acid derived from a parasite; the trigger ODNP is one strand of double-stranded genomic nucleic acid or cDNA; the trigger ODNP is an RNA molecule; the trigger ODNP is derived from nucleic acid obtained from a bacterium; the trigger ODNP is derived from nucleic acid obtained from a virus; the trigger ODNP is derived from nucleic acid obtained from a fungus; the trigger ODNP is derived from nucleic acid derived from a parasite; at least one of the deoxynucleoside triphosphate(s) is labeled; at least one of the deoxynucleoside triphosphate(s) is linked to a radiolabel; at least one of the deoxynucleoside triphosphate(s) is linked to an enzyme label at least one of the deoxynucleoside triphosphate(s) is linked to a fluorescent dye that functions as a label; at least one of the deoxynucleoside triphosphate(s) is linked to digoxidenin which functions as a label; at least one of the deoxynucleoside triphosphate(s) is linked to biotin; the same DNA polymerase type is used in all of the steps of a method; the DNA polymerase is 5′→3′ exonuclease deficient; the DNA polymerase is 5′→3′ exonuclease deficient and selected from exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase and T4 DNA polymerase homoenzyme, where any two or more of the listed DNA polymerases may be combined to form a group from which the DNA polymerase used in a method of the invention is selected, e.g., the 5′→3′ exonuclease deficient DNA polymerase is exo⁻ Bst polymerase, exo⁻ Bca polymerase, exo⁻ Vent polymerase, 9° Nm™ DNA polymerase or exo⁻ Deep Vent polymerase; the DNA polymerase has a strand displacement activity; each amplification reaction is performed in the presence of a strand displacement facilitator; a strand displacement facilitator is used during amplification, where the strand displacement facilitator is selected from the group BMRF1 polymerase accessory subunit, adenovirus DNA-binding protein, herpes simplex viral protein ICP8, single-stranded DNA binding proteins, phage T4 gene 32 protein, calf thymus helicase, and trehalose, where the invention provides that any two or more of the listed facilitators may be combined to form a group from which a facilitator is selected in order to perform an embodiment of the present invention; the strand displacement facilitator is trehalose.

Any of the methods of the present invention may, and preferably does, include the step of detecting an amplified nucleic acid, e.g., detecting the formation, either qualitatively or quantitatively, of A2. In one embodiment, the detection is performed at least partially by a technique selected from luminescence spectroscopy or spectrometry, fluorescence spectroscopy or spectrometry, mass spectrometry, liquid chromatography, fluorescence polarization, and electrophoresis, where any two, three, four, or more members of the listed techniques may be grouped together so as to form a group of techniques from which the techniques utilized in an embodiment of the present invention may be selected, e.g., the detection may performed by mass spectrometry or liquid chromatography. In one embodiment, the detection entails the use of a fluorescence-intercalating agent that specifically binds to double-stranded nucleic acid.

In other aspects, the present invention provides compositions that may be useful in, or generated by, the methods of the present invention. In one aspect, the present invention provides a composition that includes: (a) A first at least partially double-stranded nucleic acid molecule (in a first embodiment, N1 as referred to herein, while in a second embodiment, H1 as referred to herein) of which one strand comprises the nucleotide sequence of the antisense strand of a first nicking agent recognition sequence (NARS). (b) A second at least partially double-stranded nucleic acid molecule (in the first embodiment, N2 as referred to herein, while in the second embodiment, H2 as referred to herein) of which one strand comprises, from 5′ to 3′: (i) the nucleotide of the antisense strand of a second NARS, and (ii) a nucleotide sequence that is at least substantially identical, and optionally is exactly identical, to a sequence located 5′ to the nucleotide of the antisense strand of the first NARS in the first nucleic acid. Optionally, the first NARS is recognizable by a first nicking endonuclease, and the second NARS is recognizable by a second nicking endonuclease, or the first NARS is recognizable by a first restriction endonuclease, and the second NARS is recognizable by a second restriction endonuclease. The first NARS may be identical to the second NARS. Optionally, sequence (b) (ii) is exactly identical to a sequence located 5′ to the nucleotide of the antisense strand of the first NARS in the first nucleic acid. In a related composition, the present invention provides a composition that includes: (a) A first at least partially double-stranded nucleic acid molecule (in a first embodiment this molecule being N1 as described herein, while in a second embodiment this molecule is H1 as described herein) of which one strand comprises the nucleotide sequence of the sense strand of a first nicking agent recognition sequence (NARS). (b) A second at least partially double-stranded nucleic acid molecule (in the first embodiment this molecule is N2 as described herein, while in the second embodiment this molecule is H2 as described herein) of which one strand comprises from 5′ to 3′: (i) the nucleotide sequence of the antisense strand of a second NARS, and (ii) a nucleotide sequence that is at least substantially complementary to, and optionally is exactly complementary to, a nucleotide sequence located 3′ to the nucleotide sequence of the sense strand of the first NARS in the first nucleic acid. Optionally, nucleotide sequence (b) (ii) is exactly complementary to a sequence located 3′ to the nucleotide of the sense strand of the NARS in the first nucleic acid. The invention also provides a composition that includes: (a) A first, at least partially double-stranded nucleic acid molecule (in a first embodiment, this molecule is N1 as described herein, while in a second embodiment this molecule is H1 as described herein) of which one strand comprises, from 3′ to 5′: (i) a first sequence (S1′) at least 8 nucleotides in length, (ii) a sequence of an antisense strand of a first NARS, and (iii) a second sequence (S2′) that is at least 8 nucleotides in length and is not substantially identical to S1′. (b) A second, at least partially double-stranded nucleic acid molecule (optionally in the first embodiment, this molecule is N2 as described herein, while optionally in the second embodiment this molecule is H2 as described herein) of which one strand comprises, from 3′ to 5′: (i) a sequence that is at least substantially identical to, and optionally is exactly identical to, S2′, (ii) the nucleotide sequence of the antisense strand of a second NARS, and (iii) a nucleotide sequence that is at least substantially, and optionally is exactly, identical to S1′.

In these compositions, the following additional criteria and/or components may be used to describe the compositions, as well as criteria set forth above in connection with the methods of the invention: the composition further comprises a first NA that recognizes the first NARS and a second NA that recognizes the second NARS; the composition further comprises a nicking agent that recognizes both the first and second NARSs; the composition further comprises a nicking endonuclease (NE) that recognizes both the first and the second NERSs; the composition further comprises a nicking agent (NA) that recognizes both the first and the second NARSs; the composition further comprises N.BstNB I; the composition further comprises a DNA polymerase; the composition further comprises a DNA polymerase that is 5′→3′ exonuclease deficient; the composition further comprises a DNA polymerase selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase and T4 DNA polymerase homoenzyme; the composition further comprises a DNA polymerase with strand displacement activity; the composition further comprises a strand displacement facilitator; the composition further comprises a strand displacement facilitator selected from the group BMRF1 polymerase accessory subunit, adenovirus DNA-binding protein, herpes simplex viral protein ICP8, single-stranded DNA binding proteins, phage T4 gene 32 protein, calf thymus helicase, and trehalose, where one or more members of this group may be combined to form a group from which the facilitator is selected in an embodiment of the invention; the composition includes trehalose; the composition includes a labeled deoxynucleoside triphosphate. The composition includes a labeled oligonucleotide that is at least substantially complementary to, and optionally is exactly complementary to, a sequence located 5′ to the nucleotide of the antisense strand of the second NARS in T2. The composition includes a fluorescent intercalating agent.

In other aspects, the present invention provides isolated nucleic acid molecule. For instance, in one aspect, the present invention provides an isolated single-stranded nucleic acid molecule that, from 3′ to 5′, consists essentially of: (i) A sequence that is 6-100 nucleotides in length. (ii) The nucleotide sequence of the antisense strand of a nicking agent recognition sequence (NARS). (iii) A nucleotide sequence that is at most 100 nucleotides in length. This isolated nucleic acid molecule may be combined with an oligonucleotide primer (trigger ODNP) that is at least substantially complementary to, and optionally is exactly complementary to, sequence (i), so as to form a composition of the present invention. Another isolated single-stranded nucleic acid molecule provided by the present invention includes at least two nucleotide sequences that are identical to the antisense strand of a nicking agent recognition sequence (NARS). In one embodiment, the nucleic acid molecule is at most 100, or 90, or 80, or 70, or 60, or 50, or 40, or 30 nucleotides in length. Optionally, the distance between the two closest of the at least two sequences is no more than 70, or 60, or 50 or 40, or 35, or 30, or 35, or 20, or 15, or 10 nucleotides. In various aspects of this composition, one or more of the following criteria may be used to describe the composition: the trigger ODNP is exactly complementary to sequence (i); the composition also includes a nicking agent (NA) that recognizes the NARS, where the NA is optionally a nicking endonuclease (NE), where the NE is optionally N.BstNB I or N.AIw I; the compositions further contains a DNA polymerase, e.g., a DNA polymerase that is 5′→3′ exonuclease deficient, e.g., a DNA polymerase selected from exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ polymerase, and T4 DNA polymerase homoenzyme, and a DNA polymerase that has a strand displacement activity; the composition also includes a strand displacement facilitator, e.g., a strand displacement facilitator selected from the group BMRF1 polymerase accessory subunit, adenovirus DNA-binding protein, herpes simplex viral protein ICP8, single-stranded DNA binding proteins, phage T4 gene 32 protein, calf thymus helicase, and trehalose. The composition further includes trehalose. The following criteria, in any combination, may be used to further characterize the isolated nucleic acid molecules and/or the compositions that contain these nucleic acid molecules in non-isolated form: the NARS is recognizable by a nicking endonuclease (NE); the NARS is recognizable by a restriction endonuclease (RE); nucleotide sequence (i) is from 8 to 24 nucleotides in length; nucleotide sequence (i) is from 12 to 17 nucleotides in length; the isolated nucleic acid molecule is at most 200 nucleotides in length; the isolated nucleic acid molecule is at most 100 nucleotides in length; the isolated nucleic acid molecule is at most 50, or 45, or 40, or 35, or 30 nucleotides in length; a portion of sequence (iii) at the 5′ terminus of the isolated nucleic acid molecule is at least substantially identical to, and optionally is exactly identical to, a portion of sequence (i) that is at least 6 nucleotides in length; the portion of sequence (iii) at the 5′ terminus of the isolated nucleic acid molecule is exactly identical to the portion of sequence (i) that is at least 6 nucleotides in length; the isolated single-stranded nucleic acid molecule is immobilized to a substrate; the isolated single-stranded nucleic acid is covalently immobilized to the substrate; the isolated single-stranded nucleic acid is non-covalently immobilized to the substrate; the isolated single-stranded nucleic acid molecule is immobilized to a substrate formed, at least in part, from silicon, glass, paper, ceramic, metal, metalloid and plastics; the isolated single-stranded nucleic acid is immobilized to the substrate via a linker.

In other aspects, the present invention also provides arrays. For example, the present invention provides an array that includes: (a) A substrate having a plurality of distinct areas. (b) A plurality of single-stranded nucleic acids immobilized to the distinct areas wherein a single-stranded nucleic acid in the plurality is the isolated single-stranded nucleic acid molecule described herein. The following criteria may be used to further describe these arrays, where these criteria may be combined in any combination: the single-stranded nucleic acid molecules in any one of the distinct areas are homogeneous, but different from the single-stranded nucleic acid molecules in another distinct area; the single-stranded nucleic acid molecules in at least one of the distinct areas are heterogeneous; the plurality of single-stranded nucleic acids are covalently immobilized to the substrate; the plurality of single-stranded nucleic acids are non-covalently immobilized to the substrate; the substrate is made, at least in part, of a material selected from silicon, glass, paper, ceramic, metal, metalloid, and plastic. In a related aspect, the present invention provides a method for using this array, where the method amplifies one or more single-stranded nucleic acid molecules. The method includes: (A) Applying to the array as just described, (i) one or more nucleic acid amplification reaction mixtures, wherein the amplification reaction was performed in the presence of a first nicking agent, or (ii) the amplification product(s) of the amplification reaction of (i). (B) Performing an amplification reaction on the array as treated in (A), in the presence of a second nicking agent. The nucleic acid molecules that are immobilized to the substrate of the array include the antisense sequence of a NARS that is recognized by the second nicking agent. The amplification reaction amplifies one or more single-stranded nucleic acids. Optionally, in this method, the first nicking agent is identical to the second nicking agent.

In any of the methods or compounds or compositions of the present invention that include a NARS, the NARS may contain a, i.e., one or more, mismatched nucleotides. In other words, one or more of the nucleotide base pairs that form the NARS may not be hybridized according to the conventional Watson-Crick base pairing rules. However, when mismatched nucleotides are present in the NARS, then at least all of the nucleotides that are necessary to form the sense strand of the NARS are present. In one embodiment, an NARS comprises a mismatched base pair. In one embodiment, there are no mismatched base pairs in a NARS. In one embodiment, all the bases present in a NARS are matched according to conventional Watson-Crick base pairing rules. In one embodiment, there is one mismatched base pair in the NARS, while in another embodiment there are two mismatched base pairs in the NARS, while in another embodiment all of the base pairs that form the NARS are mismatched, while in another embodiment, n-1 of the base pairs that form the NARS are mismatched, where n base pairs form the NARS. In one embodiment where the invention utilizes both first and second NARSs, the mismatches present in the first NARS are also present in the second NARS. In one embodiment where the invention utilizes both first and second NARSs, the mismatches present in the first NARS are not also present in the second NARS. In one embodiment where the invention utilizes both first and second NARSs, the first NARS does not contain mismatched base pairs, however the second NARS does contain one or more mismatched base pairs. In one embodiment, there is an unmatched nucleotide in the NARS. In another embodiment, all of the nucleotides that form the sense sequence of the NARS are unmatched. In another embodiment, the NARS comprises an unmatched nucleotide.

These and other aspects of the present invention will become evident upon reference to the following detailed description and attached drawings. In addition, the various references set forth herein describe in more detail certain procedures or compositions and are therefore incorporated by reference in their entirety.

### BRIEF DESCRIPTION OF THE DRAWINGS

FIG. 1 is a schematic diagram of the major steps of the first amplification reaction of a tandem amplification system of the present invention.

FIG. 2 is a schematic diagram of the major steps of the second amplification reaction of a tandem amplification system of the present invention.

FIG. 3 is a schematic diagram of the major steps of an exemplary method for nucleic acid amplification according to the present invention, where the recognition sequence of N.BstNB I is used as an exemplary NARS, and both the first template (T1) and the second template (T2) comprise a sequence of an antisense strand of the NARS (i.e., 5′-GACTC-3′).

FIG. 4 is a schematic diagram of the major steps of an exemplary method of one-template amplification of a trigger ODNP according to the present invention, where the recognition sequence of N.BstNB I is used as an exemplary NARS.

FIG. 5 is a schematic diagram of the major steps of an exemplary method of two-template amplification of a trigger ODNP according to the present invention, where the recognition sequence of N.BstNB I is used as an exemplary NARS.

FIG. 6 is a schematic diagram of the major steps of another exemplary method for nucleic acid amplification according to the present invention, wherein NARSs recognizable by NAs that nick within their respective recognition sequences are used as exemplary NARSs. In this exemplary method, only one template (T1) that comprises sequences of antisense strands of two NARSs is needed for exponential amplification of another nucleic acid molecule (A2).

FIG. 7 is a schematic diagram of the major steps of another exemplary method of nucleic acid amplification according to the present invention, where the recognition sequence of N.BstNB I is used as an exemplary NARS, the first template (T1) comprises a sequence of a sense strand of the NARS (i.e., 5′-GAGTC-3′), and the second template (T2) comprises a sequence of an antisense strand of the NARS (i.e., 5′-GACTC-3′).

FIG. 8 shows a schematic diagram of the major steps for preparing an initial nucleic acid molecule N1 by annealing a trigger ODNP derived from a genomic DNA to a first template T1 and subsequent amplification of a single-stranded nucleic acid molecule A1.

FIG. 9 shows a schematic diagram of the major steps for preparing an initial nucleic acid molecule N1 from a genomic DNA and subsequent amplification of a single-stranded nucleic acid molecule A1. The genomic DNA comprises a nicking agent recognition sequence. The N1 molecule is produced by annealing one strand of the genomic DNA fragment to a first template (T1) that is a portion of the other strand of the genomic DNA fragment.

FIG. 10 shows a schematic diagram of the major steps for preparing an initial nucleic acid molecule N1 from a genomic DNA and subsequent amplification of a nucleic acid molecule A1. The genomic DNA comprises a nicking agent recognition sequence and a restriction endonuclease recognition sequence. A nicking endonuclease recognition sequence recognizable by a nicking endonuclease that nicks outside its recognition sequence is used as an exemplary nicking agent recognition sequence.

FIG. 11 shows a schematic diagram of the major steps for preparing an initial nucleic acid molecule N1 from a target nucleic acid using two oligonucleotide primers and subsequent amplification of a nucleic acid molecule A1. One primer comprises a sequence of a sense strand of a NERS while the other comprises one strand of a Type IIs restriction endonuclease recognition sequence (TRERS).

FIG. 12 shows a schematic diagram of the major steps for preparing initial nucleic acid molecules N1a and N1b using two ODNPs and subsequent amplification of nucleic acid molecules A1a and A1b. In this exemplary embodiment, both ODNPs comprise a sequence of the sense strand of a NERS.

FIG. 13 shows a schematic diagram of the major steps for preparing an initial nucleic acid molecule N1 in an exemplary embodiment using two ODNPs and subsequent amplification of a nucleic acid molecule A1. Both ODNPs comprise a sequence of one strand of a RERS. The amplification is performed in the presence of an α-thio deoxynucleoside triphosphate, which is used as an exemplary modified deoxynucleoside triphosphate.

FIG. 14 shows a schematic diagram of a method for detecting an immobilized target nucleic acid using a partially double-stranded initial nucleic acid molecule N1 that comprises a NARS. A NERS that is recognizable by a NE that nicks outside its recognition sequence (e.g., N.BstNB I) is used as an exemplary NARS.

FIG. 15 shows a schematic diagram of a method for detecting an immobilized target nucleic acid using a single-stranded nucleic acid molecule T1 that comprises a sequence of the antisense strand of a NARS.

FIG. 16 shows mass spectrometry analyses of an amplified DNA fragment. The top panel shows the ion current for a fragment with a mass/charge ratio of 1448.6. The middle panel shows the trace from the diode array. The bottom panel shows the total ion current from the mass spectrometer.

FIG. 17 shows mass spectrometry analyses in a control experiment. The top panel shows the trace from the diode array. The top panel shows the total ion current from the mass spectrometer. The middle panel shows the ion current for a fragment with a mass/charge ratio of 1448.6. The bottom panel shows the trace of diode array.

FIG. 18 shows the accumulation of fluorescence of a representative nucleic acid amplification reaction mixture as a function of time.

FIG. 19 shows a schematic diagram of a method for detecting the presence of a target nucleic acid in using an immobilized T1 molecule that comprises a sequence of the sense strand of a NARS and a sequence that is at least substantially complementary to, and optionally is exactly complementary to, the 3′ portion of the target nucleic acid.

FIG. 20 shows a schematic diagram of a method for detecting the presence of a target nucleic acid in using an immobilized T1 molecule that comprises a sequence of the sense strand of a NARS and is at least substantially complementary to, and optionally is exactly complementary to, the target nucleic acid.

FIG. 21 shows a schematic diagram of the major steps for preparing an initial nucleic acid molecule N1 from a target nucleic acid and subsequent amplification of a single-stranded nucleic acid molecule A1. The target nucleic acid comprises a restriction endonuclease recognition sequence and a potential genetic variation.

FIG. 22 shows a schematic diagram of the major steps for preparing an initial nucleic acid molecule N1 from a target nucleic acid and subsequent amplification of single-stranded nucleic acid molecule A1. The target nucleic acid comprises a nicking agent recognition sequence, a restriction endonuclease recognition sequence, and a genetic variation between the two recognition sequences.

FIG. 23 shows a schematic diagram of the major steps for preparing an initial nucleic acid molecule N1 from a target nucleic acid using two primers and subsequent amplification of a nucleic acid molecule A1. The target nucleic acid comprises a genetic variation (“X”). The first primer comprises a sequence of the sense strand of a nicking endonuclease recognition sequence, whereas the second primer comprises a sequence of one strand of a type IIs restriction endonuclease recognition sequence.

FIG. 24 shows a schematic diagram of the major steps for preparing initial nucleic acid molecules (and N1b) from a target nucleic acid using two primers and subsequent amplification of nucleic acid molecules A1a and A1b. The target nucleic acid comprises a genetic variation (“X”). Both primers comprise a sequence of the sense strand of a nicking endonuclease recognition sequence.

FIG. 25 shows a schematic diagram of the major steps for preparing initial nucleic acid molecules (N1a and N1b) from a target nucleic acid using two primers and subsequent amplification of nucleic acid molecules A1a and A1b. The target nucleic acid comprises a genetic variation (“X”). Both primers comprise a sequence of one strand of a restriction endonuclease recognition sequence.

FIG. 26 shows that a schematic diagram of the major steps for preparing an initial nucleic acid molecule (N1) from a target cDNA and subsequent amplification of a nucleic acid molecule (A1). The target cDNA comprises a nicking endonuclease recognition sequence, a restriction endonuclease recognition sequence, and a location suspected to be a specific exon-exon junction between the two recognition sequences.

FIGS. 27A and 27B show schematic diagrams of the process for preparing an initial nucleic acid molecule (N1) from a target cDNA and subsequent amplification of a nucleic acid molecule (A1). The target cDNA comprises Exon A and Exon B that is directly downstream to Exon A (FIG. 27A), or Exon A, Exon B, and a sequence between Exon A and Exon B (FIG. 27B).

FIG. 28 shows a schematic diagram of the major steps for preparing an initial nucleic acid molecule (N1) from a target cDNA using two primers and subsequent amplification of a nucleic acid molecule (A1). The target cDNA comprises exon A and exon B. The first primer comprises a sequence of the sense strand of a nicking endonuclease recognition sequence and anneal to a portion of the antisense strand of exon A. The second primer comprises a sequence of the antisense strand of a type IIs restriction endonuclease recognition sequence and anneals to a portion of the sense strand of exon B.

FIG. 29 shows a schematic diagram of the major steps for preparing initial nucleic acid molecules (N1a and N1b) from a target cDNA using two primers and subsequent amplification of a nucleic acid molecule (A1a and A1b). The target cDNA comprises exon A and exon B. Both primers comprise a sequence of the sense strand of a nicking endonuclease recognition sequence. The first primer anneals to a portion of the antisense strand of exon A, whereas the second primer anneals to a portion of the sense strand of exon B.

FIG. 30 shows a schematic diagram of the major steps for preparing initial nucleic acid molecules (N1a and N1b) from a target cDNA using two primers and subsequent amplification of a nucleic acid molecule (A1a and A1b). The target cDNA comprises exon A and exon B. Both primers comprise a sequence of one strand of a restriction endonuclease recognition sequence. The first primer anneals to a portion of the antisense strand of exon A, whereas the second primer anneals to a portion of the sense strand of exon B.

### DETAILED DESCRIPTION OF THE INVENTION

The present invention provides simple and efficient methods and kits for exponential amplification of nucleic acids using nicking agents. The amplification can be carried out isothermally and need not be transcription-based. These methods and kits are useful in many areas, especially in pathogen or disease diagnosis.

A. Conventions/Definitions

Prior to providing a more detailed description of the present invention, it may be helpful to an understanding thereof to define conventions and provide definitions as used herein, as follows. Additional definitions are also provided throughout the description of the present invention.

The terms “3”′ and “5”′ are used herein to describe the location of a particular site within a single strand of nucleic acid. When a location in a nucleic acid is “3′ to” or “3′ of” a reference nucleotide or a reference nucleotide sequence, this means that the location is between the 3′ terminus of the reference nucleotide or the reference nucleotide sequence and the 3′ hydroxyl of that strand of the nucleic acid. Likewise, when a location in a nucleic acid is “5′ to” or “5′ of” a reference nucleotide or a reference nucleotide sequence, this means that it is between the 5′ terminus of the reference nucleotide or the reference nucleotide sequence and the 5′ phosphate of that strand of the nucleic acid. Further, when a nucleotide sequence is “directly 3′ to” or “directly 3′ of” a reference nucleotide or a reference nucleotide sequence, this means that the nucleotide sequence is immediately next to the 3′ terminus of the reference nucleotide or the reference nucleotide sequence. Similarly, when a nucleotide sequence is “directly 5′ to” or “directly 5′ of” a reference nucleotide or a reference nucleotide sequence, this means that the nucleotide sequence is immediately next to the 5′ terminus of the reference nucleotide or the reference nucleotide sequence.

A “naturally occurring nucleic acid” refers to a nucleic acid molecule that occurs in nature, such as a full-length genomic DNA molecule or an mRNA molecule.

An “isolated nucleic acid molecule” refers to a nucleir arid molecule that is not identical to any naturally occurring nucleic acid or to that of any fragment of a naturally occurring genomic nucleic acid spanning more than three separate genes.

As used herein, a nucleotide sequence (“first sequence”), which is a portion of another nucleotide sequence (“second sequence”) located at the 5′ terminus of the other nucleotide sequence refers to a 5′ terminal sequence of the other nucleotide sequence. In other word, the 5′ terminus of the first sequence is identical to that of the second sequence.

As used herein, “nicking” refers to the cleavage of only one strand of a fully double-stranded nucleic acid molecule or a double-stranded portion of a partially double-stranded nucleic acid molecule at a specific position relative to a nucleotide sequence that is recognized by the enzyme that performs the nicking. The specific position where the nucleic acid is nicked is referred to as the “nicking site” (NS).

A “nicking agent” (NA) is an enzyme that recognizes a particular nucleotide sequence of a completely or partially double-stranded nucleic acid molecule and cleaves only one strand of the nucleic acid molecule at a specific position relative to the recognition sequence. Nicking agents include, but are not limited to, a nicking endonuclease (e.g., N.BstNB I) and a restriction endonuclease (e.g., Hinc II) when a completely or partially double-stranded nucleic acid molecule contains a hemimodified recognition/cleavage sequence in which one strand contains at least one derivatized nucleotide(s) that prevents cleavage of that strand (i.e., the strand that contains the derivatized nucleotide(s)) by the restriction endonuclease.

A “nicking endonuclease” (NE), as used herein, refers to an endonuclease that recognizes a nucleotide sequence of a completely or partially double-stranded nucleic acid molecule and cleaves only one strand of the nucleic acid molecule at a specific location relative to the recognition sequence. Unlike a restriction endonuclease (RE), which requires its recognition sequence to be modified by containing at least one derivatized nucleotide to prevent cleavage of the derivatized nucleotide-containing strand of a fully or partially double-stranded nucleic acid molecule, a NE typically recognizes a nucleotide sequence composed of only native nucleotides and cleaves only one strand of a fully or partially double-stranded nucleic acid molecule that contains the nucleotide sequence.

As used herein, “native nucleotide” refers to adenylic acid, guanylic acid, cytidylic acid, thymidylic acid or uridylic acid. A “derivatized nucleotide” is a nucleotide other than a native nucleotide.

The nucleotide sequence of a completely or partially double-stranded nucleic acid molecule that a NA recognizes is referred to as the “nicking agent recognition sequence” (NARS). Likewise, the nucleotide sequence of a completely or partially double-stranded nucleic acid molecule that a NE recognizes is referred to as the “nicking endonuclease recognition sequence” (NERS). The specific sequence that a RE recognizes is referred to as the “restriction endonuclease recognition sequence” (RERS). A “hemimodified RERS,” as used herein, refers to a double-stranded RERS in which one strand of the recognition sequence contains at least one derivatized nucleotide (e.g., α-thio deoxynucleotide) that prevents cleavage of that strand (i.e., the strand that contains the derivatized nucleotide within the recognition sequence) by a RE that recognizes the RERS.

In certain embodiments, a NARS is a double-stranded nucleotide sequence where each nucleotide in one strand of the nucleotide is complementary to the nucleotide at its corresponding position in the other strand. In such embodiments, the nucleotide of a NARS in the strand containing a NS nickable by a NA that recognizes the NARS is referred to as a “sequence of the sense strand of the NARS” or a “sequence of the sense strand of the double-stranded NARS,” while the nucleotide of the NARS in the strand that does not contain the NS is referred to as a “sequence of the antisense strand of the NARS” or a “sequence of the antisense strand of the double-stranded NARS.”

Likewise, in the embodiments where a NERS is a double-stranded nucleotide sequence of which one strand is exactly complementary to the other strand, the nucleotide of a NERS located in the strand containing a NS nickable by a NE that recognizes the NERS is referred to as a “sequence of a sense strand of the NERS” or a “sequence of the sense strand of the double-stranded NERS,” while the nucleotide of the NERS located in the strand that does not contain the NS is referred to a “sequence of the antisense strand of the NERS” or a “sequence of the antisense strand of the double-stranded NERS.” For example, the recognition sequence and the nicking site of an exemplary nicking endonuclease, N.BstNB I, are shown below with “▾” to indicate the cleavage site and N to indicate any nucleotide:

▾
5′-GAGTCNNNNN-3′
3′-CTCAGNNNNN-5′

The sequence of the sense strand of the N.BstNB I recognition sequence is 5′-GAGTC-3′, whereas that of the antisense strand is 5′-GACTC-3′.

Similarly, the sequence of a hemimodified RERS in the strand containing a NS nickable by a RE that recognizes the hemimodified RERS (i.e., the strand that does not contain any derivatized nucleotides) is referred to as “the sequence of the sense strand of the hemimodified RERS” and is located in “the sense strand of the hemimodified RERS” of a hemimodified RERS-containing nucleic acid, while the sequence of the hemimodified RERS in the strand that does not contain the NS (i.e., the strand that contains derivatized nucleotide(s)) is referred to as “the sequence of the antisense strand of the hemimodified RERS” and is located in “the antisense strand of the hemimodified RERS” of a hemimodified RERS-containing nucleic acid.

In certain other embodiments, a NARS is an at most partially double-stranded nucleotide sequence that has one or more nucleotide mismatches, but contains an intact sense strand of a double-stranded NARS as described above. According to the convention used herein, in the context of describing a NARS, when two nucleic acid molecules anneal to one another so as to form a hybridized product, and the hybridized product includes a NARS, and there is at least one mismatched base pair within the NARS of the hybridized product, then this NARS is considered to be only partially double-stranded. Such NARSs may be recognized by certain nicking agents (e.g., N.BstNB I) that require only one strand of double-stranded recognition sequences for their nicking activities. For instance, the NARS of N.BstNB I may contain, in certain embodiments, an intact sense strand, as follows,

5′-GAGTC-3′
3′-NNNNN-5′

where N indicates any nucleotide, and N at one position may or may not be identical to N at another position, however there is at least one mismatched base pair within this recognition sequence. In this situation, the NARS will be characterized as having at least one mismatched nucleotide.

In certain other embodiments, a NARS is a partially or completely single-stranded nucleotide sequence that has one or more unmatched nucleotides, but contains an intact sense strand of a double-stranded NARS as described above. According to the convention used herein, in the context of describing a NARS, when two nucleic acid molecules (i.e., a first and a second strand) anneal to one another so as to form a hybridized product, and the hybridized product includes a nucleotide sequence in the first strand that is recognized by a NA, i.e., the hybridized product contains a NARS, and at least one nucleotide in the sequence recognized by the NA does not correspond to, i.e., is not across from, a nucleotide in the second strand when the hybridized product is formed, then there is at least one unmatched nucleotide within the NARS of the hybridized product, and this NARS is considered to be partially or completely single-stranded. Such NARSs may be recognized by certain nicking agents (e.g., N.BstNB I) that require only one strand of double-stranded recognition sequences for their nicking activities. For instance, the NARS of N.BstNB I may contain, in certain embodiments, an intact sense strand, as follows,

5′-GAGTC-3′
3′-N₀₋₄-5′

(where “N” indicates any nucleotide, 0-4 indicates the number of the nucleotides “N,” a “N” at one position may or may not be identical to a “N” at another position), which contains the nucleotide of the sense strand of the double-stranded recognition sequence of N.BstNB I. In this instance, at least one of G, A, G, T or C is unmatched, in that there is no corresponding nucleotide in the complementary strand. This situation arises, e.g., when there is a “loop” in the hybridized product, and particularly when the sense sequence is present, completely or in part, within a loop.

As used herein, the phrase “amplifying a nucleic acid molecule” or “amplification of a nucleic acid molecule” refers to the making of two or more copies of the particular nucleic acid molecule. “Exponentially amplifying a nucleic acid molecule” or “exponential amplification of a nucleic acid molecule” refers to the amplification of the particular nucleic acid molecule by a tandem amplification system that comprises two or more nucleic acid amplification reactions in which the amplification product from the first amplification reaction functions as an amplification primer for the second nucleic acid amplification reaction. As used herein, the term “nucleic acid amplification reaction” refers to the process of making more than one copy of a nucleic acid molecule (A) using a nucleic acid molecule (T) that comprises a sequence complementary to the nucleotide of nucleic acid molecule A as a template. According to the present invention, both the first and the second nucleic acid amplification reactions employ nicking and primer extension reactions.

An “amplification primer,” as used herein, is an oligonucleotide that anneals to a template nucleic acid comprising a sequence of an antisense strand of a NARS and functions as a primer for an initial primer extension. The resulting extension product from the initial primer extension, that is, the strand containing the nucleotide of the amplification primer, is then nicked and the fragment in the same strand containing the 3′ terminus at the nicking site then function as a primer for subsequent primer extensions.

A “trigger oligonucleotide primer (ODNP)” is an ODNP that functions as a primer in the first nucleic acid amplification reaction of a tandem nucleic acid amplification system. It triggers exponential amplification of a nucleic acid molecule in the presence of the other required components of the system (e.g., DNA polymerase, NA, deoxynucleoside triphosphates, the template for the first amplification reaction (T1), and the template for the second amplification reaction (T2)). In certain embodiments, when the template for the first amplification reaction (T1) comprises the sequence of one strand of a NARS, the trigger ODNP may comprise the sequence of the other strand of the NARS. A trigger ODNP may be derived from a target nucleic acid or may be chemically synthesized.

A nucleic acid molecule (“first nucleic acid”) is “derived from” or “originates from” another nucleic acid molecule (“second nucleic acid”) if the first nucleic acid is either a digestion product of the second nucleic acid, or an amplification product using a portion of the second nucleic acid molecule or the complement thereof as a template. The first nucleic acid molecule must comprise a sequence that is exactly identical to, or exactly complementary to, at least a portion of the second nucleic acid.

A first nucleic acid sequence is “at least substantially identical” to a second nucleic acid sequence when the complement of the first sequence is able to anneal to the second sequence in a given reaction mixture (e.g., a nucleic acid amplification mixture). In certain preferred embodiments, the first sequence is exactly identical to the second sequence, that is, the nucleotide of the first sequence at each position is identical to the nucleotide of the second sequence at the same position, and the first sequence is of the same length as the second sequence.

A first nucleic acid sequence is “at least substantially complementary” to a second nucleic acid sequence when the first sequence is able to anneal to the second sequence in a given reaction mixture (e.g., a nucleic acid amplification mixture). In certain preferred embodiments, the first sequence is exactly or completely complementary to the second sequence, that is, each nucleotide of the first sequence is complementary to the nucleotide of the second sequence at its corresponding position, and the first sequence is of the same length as the second sequence.

As used herein, a nucleotide in one strand (referred to as the “first strand”) of a double-stranded nucleic acid located at a position “corresponding to” another position (e.g., a defined position) in the other strand (referred to as the “second strand”) of a double-stranded nucleic acid refers to the nucleotide in the first strand that is complementary to the nucleotide at the corresponding position in the second strand. Likewise, a position in one strand (referred to as the “first strand”) of a double-stranded nucleic acid corresponding to a nicking site within the other strand (referred to as the “second strand”) of a double-stranded nucleic acid refers to the position between the two nucleotides in the first strand complementary to those in the second strand between which nicking occurs.

A nucleic acid sequence (or region) is “upstream to” another nucleic acid sequence (or region) when the nucleic acid sequence is located 5′ to the other nucleic acid sequence. A nucleic acid sequence (or region) is “downstream to” another nucleic acid sequence (or region) when the nucleic acid sequence is located 3′ to the other nucleic acid sequence.

B. Methods and Compositions for Exponential Amplification of Nucleic Acids

The present invention provides methods and compositions for exponential amplification of nucleic acids using nicking endonucleases. The following sections first provide a general description of the methods, and subsequently provide descriptions of two types of nucleic acid amplification methods, compositions or kits for nucleic acid amplification, and various uses of the present methods and compositions.

1. General Description

In one aspect, the present invention provides a simple and fast method for exponential amplification of nucleic acids. It uses two or more linked amplification reactions (i.e., a tandem amplification system) catalyzed by the combination of a nicking agent (NA) and a DNA polymerase. Each amplification reaction is based on the ability of a NA to nick a double-stranded or partially double-stranded nucleic acid molecule that comprises the recognition sequence of the NA and the ability of a DNA polymerase to extend from the 3′ terminus at a nicking site (NS) of the NA.

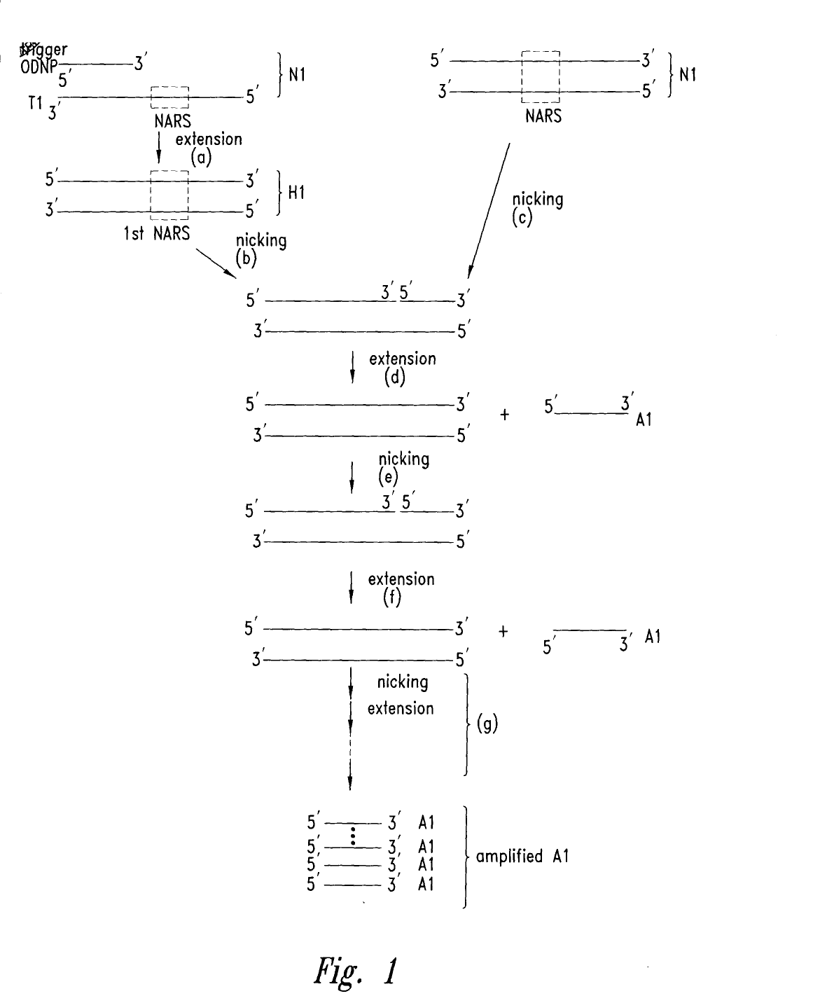

In the first amplification reaction (FIG. 1), a trigger ODNP is hybridized to a first template nucleic acid (T1) that comprises the sequence of one strand of a NARS (referred to as a “first NARS”) to form a double-stranded or partially double-stranded nucleic acid molecule (“the initial nucleic acid molecule of the first amplification reaction (N1)”). The trigger ODNP either does not contain the other strand of the first NARS and hybridizes to a portion of T1 located 3′ to the strand of the first NARS in T1, or contains the other strand of the first NARS so that its hybridization to T1 forms a nucleic acid molecule comprising a double-stranded first NARS. In either case, the nucleic acid molecule formed by the hybridization between the trigger ODNP and T1 is referred to as “the initial nucleic acid molecule of the first amplification reaction (N1).” If a portion of T1 at its 5′ terminus forms a 5′ overhang in N1, in the presence of a DNA polymerase (referred to as a “first DNA polymerase”), the trigger ODNP is extended using T1 as a template to form a hybrid (H1) that comprises the double-stranded first NARS (step (a) of FIG. 1). The resulting H1 may be nicked by a NA that recognizes the first NARS, producing a 3′ terminus and a 5′ terminus at the nicking site (step (b)). If the fragment containing the 5′ terminus at the nicking site is sufficiently short (e.g., less than 18 nucleotides in length), it will dissociate from the other portion of H1 under dissociative reaction conditions (e.g., at 60° C.). However, if this fragment does not readily dissociate, it may be displaced by the extension of the fragment from its 3′ terminus at the NS in the presence of a first DNA polymerase that is 5′→3′ exonuclease deficient and has a strand displacement activity (step (d)). Strand displacement may also occur in the absence of strand displacement activity in the first DNA polymerase, if a strand displacement facilitator is present. Such extension recreates a new NS for the first NA that can be nicked again (“re-nicked”) as in the first NA (step (e)). The fragment containing the 5′ terminus at the new NS (referred to as “A1”) may again readily dissociate from the other portion of H1 or be displaced by extension from the 3′ terminus at the NS (step (f)). The nicking-extension cycles can be repeated multiple times (step (g)), resulting in the linear accumulation/amplification of the nucleic acid fragment A1.

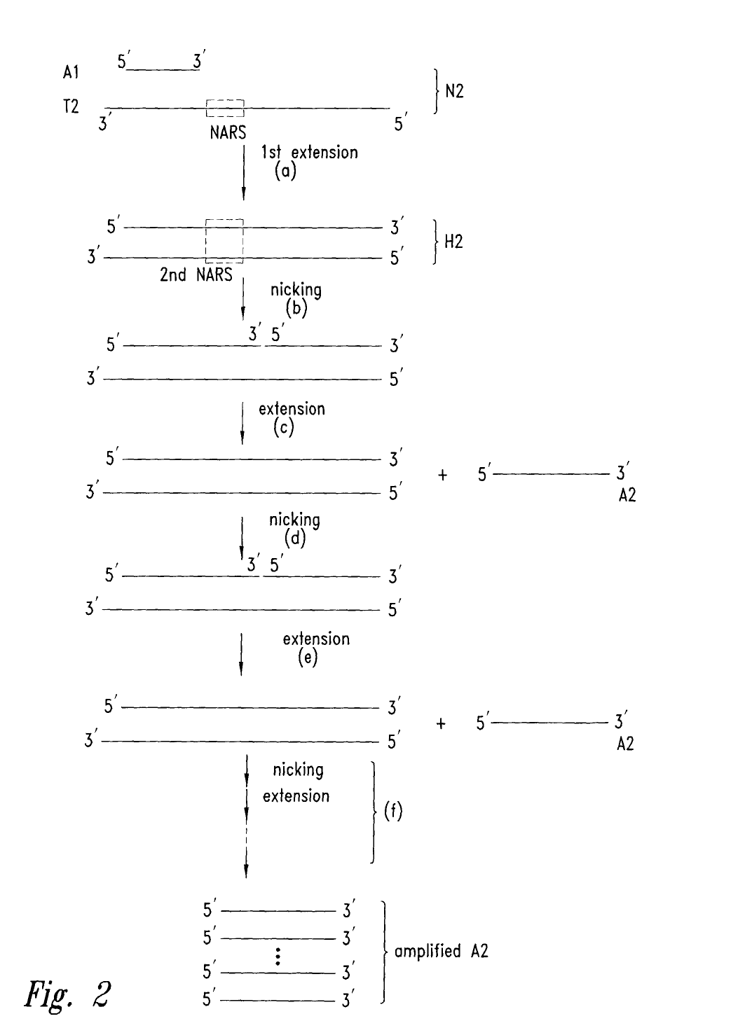

Exponential amplification of nucleic acid molecules may be performed by combining or linking the above-described first amplification reaction with another amplification reaction (referred to as “the second amplification reaction”) via the amplified fragment A1 from the first amplification reaction. In the second amplification reaction (FIG. 2), A1 hybridizes to a portion of another single-stranded nucleic acid molecule (T2) that comprises a sequence of an antisense strand of a NARS (referred to as a “second NARS”). The resulting partially double-stranded nucleic acid molecule is referred to as “the initial nucleic acid molecule of the second amplification reaction (N2).” The portion of T2 to which A1 hybridizes is located 3′ to the sequence of the antisense strand of the second NARS so that A1 functions as a primer for a primer extension reaction using T2 as a template. The extension from A1 produces a hybrid (H2) that comprises a double-stranded second NARS (step (a) of FIG. 2). In the presence of a second NA that recognizes the second NARS, H2 is nicked, producing a 3′ terminus and a 5′ terminus at the nicking site (step (b)). If the fragment containing the 5′ terminus at the nicking site is sufficiently short (e.g., less than 18 nucleotides in length), it may dissociate from the other portion of H2 under certain reaction conditions (e.g., at 60° C.). However, if this fragment does not readily dissociate from the other portion of H2, it may be displaced by extension of the fragment having a 3′ terminus at the NS in the presence of a DNA polymerase (referred to as a “second DNA polymerase”) that is 5′→3′ exonuclease deficient and has a strand displacement activity (step (c)). Strand displacement may also occur in the absence of the strand displacement activity of the second DNA polymerase, but in the presence of a strand displacement facilitator. Such extension recreates a new NS for the second NA that can be nicked again (“re-nicked”) by the second NA (step (d)). The fragment containing the 5′ terminus at the new NS (referred to as “A2”) may again readily dissociate from the other portion of H2 or be displaced by extension from the 3′ terminus at the NS (step (e)). The nicking-extension cycles can be repeated multiple times (step (f)), resulting the exponential accumulation/amplification of the nucleic acid fragment A2. The amplified single-strand nucleic acid fragment (i.e., A2), as described in detail below, may be identical to A1, different from A1, or complementary to A1.

The present method of nucleic acid amplification is not limited to linking two nucleic acid amplification reactions together. In certain embodiments, a second amplification reaction may be further linked to a third amplification reaction. In other words, the nucleic acid molecule A2 amplified during the second amplification reaction may anneal to a portion of another nucleic acid molecule “T3” that comprises the sequence of one strand of a NARS (referred to as a “third NARS”) to trigger the amplification of a nucleic acid molecule “A3” in a third amplification reaction. Additional amplification reactions may be added to the chain. For example, A3 may in turn anneal to a portion of another nucleic acid molecule “T4” also comprising one strand of a NARS (referred to as a “fourth NARS) and trigger the amplification of a nucleic acid molecule “A4” in a fourth amplification reaction. Because each subsequent amplification reaction results in a linear amplification of the amplified fragment from its previous amplification reaction, the greater number of the amplification reactions in an amplification system, the higher level of amplification, provided that the other components of the system (e.g., template nucleic acid molecules, NAs, and DNA polymerases) do not limit the amplification rate or level.

a. Nicking Agents

As described above, the exponential nucleic acid amplification method of the present invention links two or more nucleic acid amplification reactions together and each amplification reaction is performed in the presence of a NA. The NA for one amplification reaction may or may not be different from that for another amplification reaction. In one embodiment, the NAs for different amplification reactions are identical to each other, so that only one NA is required for exponential amplification of a nucleic acid molecule. In another embodiment, two different NAs, e.g., two NAs recognizing different NARSs, are employed.

Any enzyme that recognizes a specific nucleotide sequence of a fully or partially double-stranded nucleic acid and cleaves only one strand of the nucleic acid may be used as a nicking agent in the present invention. Such an enzyme can be a NE that recognizes a specific sequence that consists of native nucleotides or a RE that recognizes a hemimodified recognition sequence.

A nicking endonuclease may or may not have a nicking site that overlaps with its recognition sequence. An exemplary NE that nicks outside its recognition sequence is N.BstNB I, which recognizes a unique nucleic acid sequence composed of 5′-GAGTC-3′, but nicks four nucleotides beyond the 3′ terminus of the recognition sequence. The recognition sequence and the nicking site of N.BstNB I are shown below with “▾” to indicate the cleavage site where the letter N denotes any nucleotide:

▾
5′-GAGTCNNNNN-3′
3′-CTCAGNNNNN-5′

N.BstNB I may be prepared and isolated as described in U.S. Pat. No. 6,191,267, incorporated herein by reference in its entirety. Buffers and conditions for using this nicking endonuclease are also described in the '267 patent. An additional exemplary NE that nicks outside its recognition sequence is N.AIwI, which recognizes the following double-stranded recognition sequence:

▾
5′-GGATCNNNNN-3′
3′-CCTAGNNNNN-5′

The nicking site of N.AIwI is also indicated by the symbol “▾”. Both NEs are available from New England Biolabs (NEB). N.AIwI may also be prepared by mutating a type IIs RE AIwI as described in Xu et al. (Proc. Natl. Acad. Sci. USA 98:12990-5, 2001).

Exemplary NEs that nick within their NERSs include N.BbvCI-a and N.BbvCI-b. The recognition sequences for the two NEs and the NSs (indicated by the symbol “▾”) are shown as follows:

```
N.BbvCl-a
          ▾
5′-CCTCAGC-3′
3′-GGAGTCG-5′
N.BbvCl-b
          ▾
5′-GCTGAGG-3′
3′-CGACTCC-5′
```

Both NEs are available from NEB.

Additional exemplary nicking endonucleases include, without limitation, N.BstSE I (Abdurashitov et al., Mol. Biol. (Mosk) 30:1261-7,1996), an engineered EcoR V (Stahl et al., Proc. Natl. Acad. Sci. USA 93: 6175-80,1996), an engineered Fok I (Kim et al., Gene 203: 43-49,1997), endonuclease V from Thermotoga maritima (Huang et al., Biochem. 40: 8738-48, 2001), Cvi Nickases (e.g., CviNY2A, CviNYSI, Megabase Research Porducts, Lincoln, Nebr.) (Zhang et al., Virology 240: 366-75,1998; Nelson et al., Biol. Chem. 379: 423-8, 1998; Xia et al., Nucleic Acids Res. 16: 9477-87,1988), and an engineered Mly I (i.e., N.Mly I) (Besnier and Kong, EMBO Reports 2: 782-6, 2001). Additional NEs may be obtained by engineering other restriction endonuclease, especially type IIs restriction endonucleases, using methods similar to those for engineering EcoR V, AIwI, Fok I and/or Mly I.

A RE useful as a nicking agent can be any RE that nicks a double-stranded nucleic acid at its hemimodified recognition sequences. Exemplary REs that nick their double-stranded hemimodified recognition sequences include, but are not limited to Ava I, Bsl I, BsmA I, BsoB I, Bsr I, BstN I, BstO I, Fnu4H I, Hinc II, Hind III and Nci I. Additional REs that nick a hemimodified recognition sequence may be screened by the strand protection assays described in U.S. Pat. No. 5,631,147.

In certain embodiments, a nicking agent may recognize a nucleotide sequence in a DNA-RNA duplex and nicks in one strand of the duplex. In certain other embodiments, a nicking agent may recognize a nucleotide sequence in a double-stranded RNA and nicks in on strand of the RNA.

Certain nicking agents require only the presence of the sense strand of a double-stranded recognition sequence in an at least partially double-stranded substrate nucleic acid for their nicking activities. For instance, N.BstNB I is active in nicking a substrate nucleic acid that comprises, in one strand, the sequence of the sense strand of its recognition sequence “5′-GAGTC-3”′ of which one or more nucleotides do not form conventional base pairs (e.g., G:C, A:T, or A:U) with nucleotides in the other strand of the substrate nucleic acid. The nicking activity of N.BstNB I decreases with the increase of the number of the nucleotides in the sense strand of its recognition sequence that do not form conventional base pairs with any nucleotides in the other strand of the substrate nucleic acid. However, even none of the nucleotides of “5′-GAGTC-3”′ form conventional base pairs with the nucleotides in the other strand, N.BstNB I may still retain 10-20% of its optimum activity.

b. DNA Polymerases

As described above, the exponential nucleic acid amplification method of the present invention links two or more nucleic acid amplification reactions together and each amplification reaction is performed in the presence of a DNA polymerase. The DNA polymerase for one amplification reaction may be different from that for another amplification reaction. In one embodiment, the DNA polymerases for different amplification reactions are identical to each other, so that only one DNA polymerase is required for exponential amplification of a nucleic acid molecule.

The DNA polymerase useful in the present invention may be any DNA polymerase that is 5′→3′ exonuclease deficient but has a strand displacement activity. Such DNA polymerases include, but are not limited to, exo⁻ Deep Vent, exo⁻ Bst, exo⁻ Pfu, and exo⁻ Bca. Additional DNA polymerase useful in the present invention may be screened for or created by the methods described in U.S. Pat. No. 5,631,147, incorporated herein by reference in its entirety. The strand displacement activity may be further enhanced by the presence of a strand displacement facilitator as described below.

Alternatively, in certain embodiments, a DNA polymerase that does not have a strand displacement activity may be used. Such DNA polymerases include, but are not limited to, exo⁻ Vent, Taq, the Klenow fragment of DNA polymerase I, T5 DNA polymerase, and PHi9 DNA polymerase. In certain embodiments, the use of these DNA polymerases requires the presence of a strand displacement facilitator. A “strand displacement facilitator” is any compound or composition that facilitates strand displacement during nucleic acid extensions from a 3′ terminus at a nicking site catalyzed by a DNA polymerase. Exemplary strand displacement facilitators useful in the present invention include, but are not limited to, BMRF1 polymerase accessory subunit (Tsurumi et al., J. Virology 67: 7648-53,1993), adenovirus DNA-binding protein (Zijderveld and van der Vliet, J. Virology 68: 1158-64,1994), herpes simplex viral protein ICP8 (Boehmer and Lehman, J. Virology 67: 711-5,1993; Skaliter and Lehman, Proc. Natl. Acad. Sci. USA 91: 10665-9,1994), single-stranded DNA binding protein (Rigler and Romano, J. Biol. Chem. 270: 8910-9,1995), phage T4 gene 32 protein (Villemain and Giedroc, Biochemistry 35:14395-4404,1996), calf thymus helicase (Siegel et al., J. Biol. Chem. 267:13629-35,1992) and trehalose. In one embodiment, trehalose is present in the amplification reaction mixture.

Additional exemplary DNA polymerases useful in the present invention include, but are not limited to, phage M2 DNA polymerase (Matsumoto et al., Gene 84: 247,1989), phage PhiPRD1 DNA polymerase (Jung et al., Proc. Natl. Acad. Sci. USA 84: 8287,1987), T5 DNA polymerase (Chatterjee et al., Gene 97:13-19,1991), Sequenase (U.S. Biochemicals), PRD1 DNA polymerase (Zhu and Ito, Biochim. Biophys. Acta. 1219: 267-76,1994), 9° Nₘ™ DNA polymerase (New England Biolabs) (Southworth et al., Proc. Natl. Acad. Sci. 93: 5281-5,1996; Rodriquez et al., J. Mol. Biol. 302: 447-62, 2000), and T4 DNA polymerase holoenzyme (Kaboord and Benkovic, Curr. Biol. 5:149-57,1995).

Alternatively, a DNA polymerase that has a 5′→3′ exonuclease activity may be used. For instance, such a DNA polymerase may be useful for amplifying short nucleic acid fragments that automatically dissociate from the template nucleic acid after nicking.

In certain embodiments where a nicking agent nicks in the DNA strand of a RNA-DNA duplex, a RNA-dependent DNA polymerase may be used. In other embodiments where a nicking agent nicks in the RNA strand of a RNA-DNA duplex, a DNA-dependent DNA polymerase that extends from a DNA primer, such as Avian Myeloblastosis virus reverse transcriptase (Promega) may be used. In both instances, a target mRNA need not be reverse transcribed into cDNA and may be directly mixed with a template nucleic acid molecule that is at least substantially complementary to the target mRNA.

c. Reaction Conditions

The exponential nucleic acid amplification method of the present invention links two or more nucleic acid amplification reactions where each utilizes nicking and primer extension reactions in achieving amplification. According to the methods of the present invention, in each amplification reaction, a DNA polymerase may be mixed with nucleic acid molecules (e.g., template nucleic acid molecules) before, after, or at the same time as, a NA is mixed with the template nucleic acid. Preferably, the nicking-extension reaction buffer is optimized to be suitable for both the NA and the DNA polymerase. For instance, if N.BstNB I is the NA and exo⁻ Vent is the DNA polymerase, the nicking-extension buffer can be 0.5×N.BstNB I buffer and 1×DNA polymerase Buffer. Exemplary 1×N.BstNB I buffer may be 10 mM Tris-HCl, 10 mM MgCl₂, 150 mM KCl, and 1 mM dithiothreitol (pH 7.5 at 25° C.). Exemplary 1×DNA polymerase buffer may be 10 mM KCl, 20 mM Tris-HCl (pH 8.8 at 25° C.), 10 mM (NH₄)₂SO₄, 2 mM MgSO₄, and 0.1% Triton X-100. One of ordinary skill in the art is readily able to find a reaction buffer for a NA and a DNA polymerase.

In addition, in certain embodiments where a DNA polymerase is dissociative (i.e., the DNA polymerase is relatively easy to dissociate from a template nucleic acid, such as Vent DNA polymerase), the ratio of a NA to a DNA polymerase in a reaction mixture may also be optimized for maximum amplification of full-length nucleic acid molecules. As used herein, a “full-length” nucleic acid molecule refers to an amplified nucleic acid molecule that contains the sequence complementary to the 5′ terminal sequence of its template. In other words, a full-length nucleic acid molecule is an amplification product of a complete gene extension reaction. In a reaction mixture where the amount of a NA is excessive with respect to that of a DNA polymerase, partial amplification products may be produced. The production of partial amplification products may be due to excessive nicking of partially amplified nucleic acid molecules by the NA and subsequent dissociation of these molecules from their templates. Such dissociation prevents the partially amplified nucleic acid molecules from being further extended.

Because different NAs or different DNA polymerases may have different nicking or primer extension activities, the ratio of a particular NA to a specific DNA polymerase that is optimal to maximum amplification of full-length nucleic acids will vary depending on the identities of the specific NA and DNA polymerase. However, for a given combination of a particular NA and a specific DNA polymerase, the ratio may be optimized by carrying out exponential nucleic acid amplification reactions in reaction mixtures having different NA to DNA polymerase ratios and characterizing amplification products thereof using techniques known in the art (e.g., by liquid chromatography or mass spectrometry). The ratio that allows for maximum production of full-length nucleic acid molecules may be used in future amplification reactions.

It is noteworthy that although partial amplification of nucleic acid molecules may occur during both the first and the second amplification reactions, partial amplification during the first amplification reaction usually does not significantly affect the overall nucleic acid amplification level or rate. Because the nucleic acid molecules amplified during the first amplification reaction are used as ODNPs for initial primer extensions during the second amplification reaction, they are sufficient for their intended use if they are long enough to allow for their specific annealing to their templates. Besides using the optimal ratio of a NA to a dissociative DNA polymerase for full-length nucleic acid amplification, alternatively, the amount of partial amplification products may be eliminated or reduced by inactivating the NA but not the DNA polymerase (e.g., by heat inactivation) after amplification reactions have proceeded for a period of time and allowing each gene extension reaction to proceed to its completion.

In certain preferred embodiments, nicking and extension reactions of the present invention are performed under isothermal conditions. As used herein, “isothermally” and “isothermal conditions” refer to a set of reaction conditions where the temperature of the reaction is kept essentially constant (i.e., at the same temperature or within the same narrow temperature range wherein the difference between an upper temperature and a lower temperature is no more than 20° C.) during the course of the amplification. An advantage of the amplification method of the present invention is that there is no need to cycle the temperature between an upper temperature and a lower temperature. Both the nicking and the extension reaction will work at the same temperature or within the same narrow temperature range. If the equipment used to maintain a temperature allows the temperature of the reaction mixture to vary by a few degrees, such a fluctuation is not detrimental to the amplification reaction. Exemplary temperatures for isothermal amplification include, but are not limited to, any temperature between 50° C. to 70° C. or the temperature range between 50° C. to 70° C., 55° C. to 70° C., 60° C. to 70° C., 65° C. to 70° C., 50° C. to 55° C., 50° C. to 60° C., or 50° C. to 65° C. Many NAs and DNA polymerases are active at the above exemplary temperatures or within the above exemplary temperature ranges. For instance, both the nicking reaction using N.BstNB I (New England Biolabs) and the extension reaction using exo⁻ Bst polymerases (BioRad) may be carried out at about 55° C. Other polymerases that are active between about 50° C. and 70° C. include, but are not limited to, exo⁻ Vent (New England Biolabs), exo⁻ Deep Vent (New England Biolabs), exo⁻ Pfu (Strategene), exo⁻ Bca (Panvera) and Sequencing Grade Taq (Promega).

d. Initial Nucleic Acids (N1s)

As discussed above, the initial nucleic acid for the first nucleic acid amplification (i.e., N1) may be provided by annealing a trigger ODNP with a template nucleic acid molecule T1. Because the trigger ODNP functions as a primer for primer extension using T1 as a template, it must be substantially complementary to a portion of T1 and also have a 3′ terminus, from which primer extension occurs.

In certain embodiments, the trigger ODNP is derived from a nucleic acid molecule. The 3′ terminus of the trigger may be produced by various methods known in the art. For instance, the 3′ terminus of a trigger ODNP may be provided by digesting a nucleic acid fragment having a restriction endonuclease recognition sequence (RERS) using a restriction endonuclease that recognizes the RERS (e.g., a type IIs restriction endonuclease). The RERS in the nucleic acid fragment may be naturally occurring or may be incorporated into the fragment by using a primer that comprises one strand of the RERS. Alternatively, the 3′ terminus of a trigger ODNP may be produced by nicking a nucleic acid fragment having a NARS with a NA that recognizes the NARS. The NARS may also be naturally occurring or may be incorporated into the fragment by using a primer that comprises one strand of the NARS. In addition, the 3′ terminus of a trigger ODNP may be created by oligonucleotide-directed cleavage according to Szybalski (U.S. Pat. No. 4,935,357) or by base-specific chemical cleavage according to Maxam-Gilbert (Proc. Natl. Acad. Sci. USA 74:560-4,1977). In certain embodiments, the 3′ terminus of a trigger ODNP may be provided by cleaving a nucleic acid molecule with DNase I or other non-specific nucleases or by shearing a nucleic acid molecule. In situations where the cleavage product is a double-stranded nucleic acid, a trigger ODNP may be obtained by denaturing the double-stranded nucleic acid.

The nucleic acid molecule from which the trigger ODNP is derived may be naturally occurring or synthetic. It may be RNA or DNA, single-stranded or double-stranded. Such nucleic acid molecules include genomic DNA, cDNA or its derivates, such as randomly primed or specifically primed amplification products. The trigger ODNP itself may be a single-stranded DNA molecule or a single-stranded RNA molecule.

Likewise, T1 may also be derived from another nucleic acid molecule by enzymatic, chemical, or mechanic cleavages. Enzymatic cleavages may be accomplished, for example, by digesting the nucleic acid molecule with a restriction endonuclease that recognizes a specific sequence within the nucleic acid molecule. Alternatively, enzymatic cleavages may be accomplished by nicking the nucleic acid molecule with a nicking agent that recognizes a specific sequence within the nucleic acid molecule. Enzymatic cleavages may also be oligonucleotide-directed cleavages according to Szybalski (U.S. Pat. No. 4,935,357) or a partially double-stranded nucleic acid comprising a recognition sequence of a type IIs restriction endonuclease as described in the U.S. application entitled “Amplification of Nucleic Acid Fragments Using Nicking Agents” (Express Mail No. EV065004868US). Chemical and mechanic cleavages may be accomplished by any method known in the art suitable for cleaving nucleic acid molecules such as shearing. In situations where the cleavage product is a double-stranded nucleic acid molecule, a T1 molecule may be obtained by denaturing the double-stranded nucleic acid molecule.

As noted above, T1 contains at least a sequence of one strand of a NARS. The NARS may be present in the nucleic acid molecule from which T1 is derived. Alternatively, it may be incorporated into T1, for example, by using an ODNP comprising a sequence of one strand of the NARS. In certain embodiments, T1 may contain the sequences of one strand (i.e., sense or antisense strand, independently selected) of two or more NARSs. Typically, a trigger ODNP or a portion thereof is substantially complementary to a sequence 3′ to the strand of the NARS located most closely to the 3′ terminus of T1 (referred to as “the first NARS”). In certain embodiments, the sequences of the multiple NARSs present in a single T1 are separated by one or more nucleotides. In other embodiments, they may be directly next to each other or even partially overlap. If multiple NARSs are present in a T1 molecule, they may be identical to, or different from, each other. If the multiple NARSs are identical to each other, then in the presence of a NA that recognizes the NARSs and a DNA polymerase, the sequences between NARSs, as well as the sequence 5′ to the NARS located most closely to the 5′ terminus of T1 (referred to as “the last NARS”), are either used as templates for amplifying their complementary sequences (when T1 contains the sequence of the antisense strand of the NARSs), or amplified (when T1 contains the sequence of the sense strand of the NARSs). In certain preferred embodiments, the sequences between NARSs, as well as the sequence 5′ to the last NARS are identical to each other. In the embodiments where the NARSs are different from each other, amplification of a sequence complementary to a particular region of T1 (when T1 contains the sequence of the antisense strand of the NARSs) or of a particular region of T1 (when T1 contains the sequence of the sense strand of the NARSs) may be accomplished by selecting a NA that recognizes the NARS upstream of the particular region of T1.

Similar to trigger ONDPs, T1 molecules may be derived from various nucleic acid molecules. These nucleic acid molecules include naturally occurring nucleic acids and synthetic nucleic acids, either of which may be double-stranded or single-stranded nucleic acid molecules, and may be DNAs (such as genomic DNA and cDNA) or RNAs.

In certain embodiments, a T1 molecule comprises or consists essentially of, from 3′ to 5′: a first sequence that is at most 100 nucleotides in length; a sequence of one strand of a double-stranded nicking agent recognition sequence; and a second sequence that is at most 100 nucleotides in length. In some embodiments, a T1 molecule is at most 200, 150, 100, 80, 60, 50, 40, 30, 25, 20, 18, 16, 14, 12, or 10 nucleotides in length. The first sequence, the second sequence, or both, in certain embodiments, may be at most 100, 80, 60, 50, 40, 30, 25, 20, 18, 16, 14, 12, 10, 9, 8, 7, 6, 5, or 4 nucleotides in length.

In certain embodiments where (1) a T1 comprises a sequence of the sense strand of a nicking agent recognition sequence and (2) a trigger ODNP is complementary to a portion of the T1 molecule that flanks the sequence of the sense strand of the nicking agent recognition sequence, there may be mismatches between one or more nucleotides within the sense strand of the nicking agent recognition sequence in the T1 and the corresponding nucleotides in the trigger ODNP. In other words, one or more nucleotides within the sense strand of the nicking agent recognition sequence in the T1 may not form conventional base pair(s) with any nucleotides in the trigger ODNP. Because certain nicking agents (e.g., N.BstNB I) are capable of nicking a substrate that comprises only the sense strand of their double-stranded recognition sequences, the initial nucleic acid (N1) formed by annealing the trigger ODNP to the T1 may be used as a template to amplify a single-stranded nucleic acid (A1) in the presence of a nicking agent that recognizes the sense strand of the recognition sequence in the T1 molecule. The detailed descriptions for the use of a nicking agent to amplify a single-stranded nucleic acid using a template nucleic acid that comprises only the sequence of the sense strand, not the intact antisense strand, of a double-stranded nicking agent recognition sequence are provided in the U.S. application entitled to “Amplification of Nucleic Acid Fragments Using Nicking Agents” (Express Mail No. EV065004868US).

Alternative to the embodiments where a trigger ODNP, T1, or both are derived from a nucleic acid molecule, the present invention also includes embodiments where the trigger ODNP, T1, or both are synthetic nucleic acid molecules. Any methods known in the art for oligonucleotide synthesis may be used to synthesize trigger ODNP and/or T1. For instance, trigger ODNP and/or T1 may be synthesized by the solid phase oligonucleotide synthesis methods disclosed in U.S. Pat. Nos. 6,166,198, 6,043,353, 6,040,439, and 5,945,524 (incorporated herein in their entireties by reference). Briefly, solid phase oligonucleotide synthesis can be performed by sequentially linking 5′ blocked nucleotides to a nascent oligonucleotide attached to a resin, followed by oxidizing and unblocking to form phosphate diester linkages. In addition, the trigger ODNP and/or T1 may be purchased from companies that synthesize customer-designed oligonucleotides.

In certain embodiments, the initial nucleic acid molecule of the first amplification reaction (i.e., N1) may be provided other than by annealing a trigger ODNP with a template nucleic acid molecule T1. For instance, N1 may be a double-stranded nucleic acid molecule comprising a double-stranded NARS, which can be readily nicked by a NA that recognizes the NARS (step (c) of FIG. 1) without any initial primer extension reaction (e.g., step (a) of FIG. 1). In such a case, each strand of the N1 molecule comprises a sequence of one strand of a NARS. Thus, either strand may be regarded as a T1 molecule with its complementary strand as a trigger ODNP. A double-stranded N1 molecule may be, for example, a digestion product of a nucleic acid comprising a NARS. The sequence of NARS in N1 may be originated or derived from another nucleotide sequence, or incorporated into N1 by an oligonucleotide primer comprising the sequence of one strand of the NARS or during the chemical synthesis of T1.

N1 may be a partially double-stranded nucleic acid molecule comprising either a double-stranded NARS or only one strand of a NARS. For instance, N1 may be a nicked product of a nucleic acid molecule comprising two NARSs or a nicking digestion product of a nucleic acid molecule comprising both a NARS and a RERS.

e. T2 Molecules

Similar to T1, T2 may also be derived from another nucleic acid molecule by enzymatic, chemical or mechanic cleavages within the other nucleic acid molecule as described above, or by nucleic acid amplification using the other nucleic acid molecule as a template. The other nucleic acid molecule from which T2 is derived may be naturally occurring nucleic or synthetic, double-stranded or single-stranded nucleic acid, DNA (such as genomic DNA and cDNA) or RNA. In one embodiment T2 is chemically synthesized.

As described above, T2 contains at least a sequence of an antisense strand of a NARS. The NARS may be present in the nucleic acid molecule from which T2 is derived. Alternatively, it may be incorporated into T2, for example, by using an ODNP comprising a sequence of one strand of the NARS. In certain embodiments, T2 may contain the sequences of the antisense strands of two or more NARSs. Typically, a trigger ODNP or a portion thereof is substantially complementary to the sequence 3′ to the NARS located most closely to the 3′ terminus of T2 (referred to as “the first NARS”). In certain embodiments, the sequences of the NARSs of T2 are separated by one or more nucleotides. In other embodiments, they may be directly next to each other or even partially overlapping. If the sequences of the antisense strands of multiple NARSs are present in a T2 molecule, they may be identical to, or different from, each other. If the NARSs are identical to each other, then in the presence of a NA that recognizes the NARSs and a DNA polymerase, the sequences between NARSs, as well as the sequence 5′ to the NARS located most closely to the 5′ terminus of T2 (referred to as “the last NARS”), are used as templates for amplifying their complementary sequences. In certain preferred embodiments, the sequences between NARSs, as well as the sequence 5′ to the last NARS are identical to each other. In the embodiments where the NARSs are different from each other, amplification of a sequence complementary to a particular region of T2 may be accomplished by selecting a NA that recognizes the NARS upstream of the particular region of T2.

The number of T2 molecules in an amplification reaction mixture is typically more than that of T1 molecules. The preference for a greater number of T2 molecules than T1 molecules is due to the fact that T2 molecules are used as annealing partners for the single-stranded nucleic acid molecules (i.e., A1) amplified using T1 molecules as templates. In other words, during the first amplification reaction, each T1 molecule is used as a template to produce multiple copies of A1. Thus, for each of the T1 molecules, multiple T2 molecules are preferably present to provide annealing partners for the multiple A1 molecules amplified using a single T1 molecule as a template.

In certain embodiments, a T2 molecule comprises or consists essentially of, from 3′ to 5′: a first sequence that is at most 100 nucleotides in length; a sequence of the antisense strand of a double-stranded nicking agent recognition sequence; and a second sequence that is at most 100 nucleotides in length. In some embodiments, a T2 molecule is at most 200, 150, 100, 80, 60, 50, 40, 30, 25, 20, 18, 16, 14, 12, or 10 nucleotides in length. The first sequence, the second sequence, or both, in certain embodiments, may be at most 100, 80, 60, 50, 40, 30, 25, 20, 18, 16, 14, 12, 10, 9, 8, 7, 6, 5, or 4 nucleotides in length.

In one aspect, the present invention provides a method for amplifying a nucleic acid molecule (A2) comprising (a) providing a single-stranded nucleic acid molecule (A1); (b) providing a second single-stranded nucleic acid molecule (T2) comprising, from 5′ to 3′ (i) a nucleotide sequence termed a “template nucleotide sequence”, (ii) a sequence of an antisense strand of a NARS, and (iii) a sequence that is at least substantially complementary to A1; and (c) amplifying a third single-stranded nucleic acid molecule (A2) in the presence of T2, A1, a nicking agent that recognizes the NARS, a DNA polymerase, and one or more deoxynucleoside triphosphate(s), where A2 is complementary to at least a portion of the template nucleotide sequence of T2. Exemplary means by which A1 may be provided are described herein.

Although the exponential nucleic acid amplification method of the present invention requires that T2 comprises a sequence of an antisense strand of a NARS, T1 may comprise a sequence of a sense strand or an antisense strand of a NARS. These two types of nucleic acid amplification reactions, as well as certain preferred embodiments, are described in detail below.

2. The First Type of Nucleic Acid Amplification

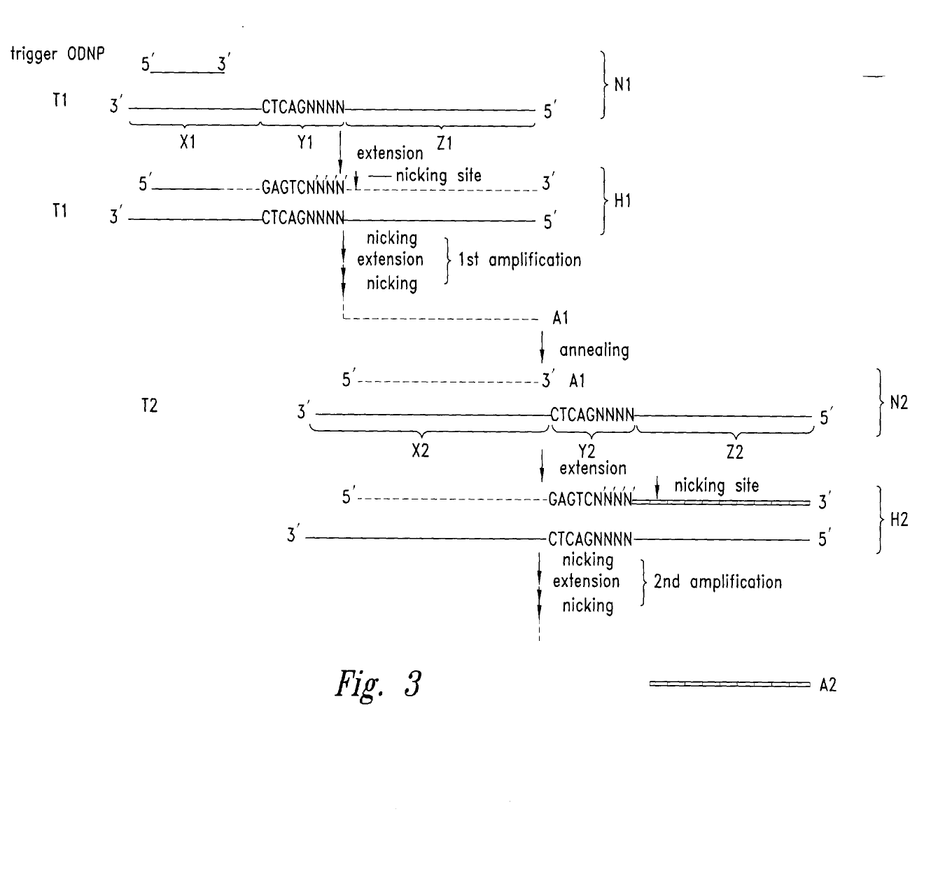

The first type of nucleic acid amplification according to the present invention is where both T1 and T2 comprise a sequence of an antisense strand of a NARS. Preferably, the sequence of the antisense strand of the NARS in T1 is identical to that in T2. Using N.BstNB I as an exemplary NA the recognition sequence of which is present in both T1 and T2, this type of nucleic acid amplification is illustrated in FIG. 3. However, one of ordinary skill in the art appreciates that other nicking agents, such as nicking endonucleases other than N.BstNB I and restriction endonucleases, may be used instead.

In the exemplary embodiment shown in FIG. 3, the initial nucleic acid molecule N1 is a partially double-stranded nucleic acid molecule formed by annealing a trigger ODNP with T1 having three regions: Regions X1, Y1 and Z1. Regions X1, Y1 and Z1 are defined as the region directly 3′ to the sequence of the antisense strand of the N.BstNB I recognition sequence, the region from the 3′ terminus of the sequence of the antisense strand of the recognition sequence of N.BstNB I to the nucleotide corresponding to the 3′ terminal nucleotide at the nicking site of N.BstNB I within the extension product of the trigger ODNP (i.e., 3′-CACAGNNNN-5′ where N can be A, T, G or C), and the region directly 5′ to Region Y1, respectively. The trigger ODNP is at least substantially complementary to Region X1 and functions as a primer for nucleic acid extension in the presence of a DNA polymerase. The extension of the trigger produces a double-stranded nucleic acid fragment (H1) or a partially double-stranded nucleic acid fragment (H1), depending on whether the 5′ terminal sequence of the trigger ODNP anneals to the 3′ terminal sequence of Region X1. The resulting H1 comprises the double-stranded N.BstNB I recognition sequence, which can be nicked by N.BstNB I. In a preferred embodiment, the 3′ terminus of T1 is blocked, for example by a phosphate group, so that the extension from this terminus is prevented. The nicked product comprising the sequence of the trigger ODNP may be extended again from its 3′ terminus at the nicking site by the DNA polymerase, displacing the strand A1 containing the 5′ terminus produced by N.BstNB I at the nicking site. The nicking-extension cycle is repeated multiple times, resulting in the accumulation of the displaced strand A1. A1 is then annealed to Region X2 of T2, which also has two additional regions: Regions Y2 and Z2, to form an initial nucleic acid molecule N2 for the second amplification reaction. Region Y2 has a similar sequence as Region Y1 (i.e., 3′-CTCAGNNNN-5′ where the Ns in Region Y2 may be identical to, or different from, those at the same positions in Region Y1), whereas Regions X2 and Z2 refer to regions immediately next to the 3′ terminus and the 5′ terminus of Region Y2, respectively. The extension of A1 using T2 as a template produces a double-stranded nucleic acid fragment (H2) or a partially double-stranded nucleic acid fragment (H2), depending on whether the 5′ terminal sequence of A1anneals to the 3′ terminal sequence of Region X2. The resulting H2 comprises the double-stranded N.BstNB I recognition sequence, which can be nicked by N.BstNB I. The 3′ terminus at the nicking site may be extended again by the DNA polymerase, displacing the strand A2 containing the 5′ terminus at the nicking site. The nicking-extension cycle is repeated multiple times, resulting in the accumulation/amplification of the displaced strand A2. The amplification of A2 is exponential because it is the final amplification product of two linked linear amplification reactions.

Because A2 is amplified using Region Z2 as a template, A2 may be designed to have an at least substantially identical sequence to, or a different sequence from, A1 by designing Region Z2 to have a sequence that is at least substantially complementary to A1 or a sequence that is not substantially complementary to A1. In one embodiment Region Z2 is at least substantially complementary to A1, so that both Regions X2 and Z2 may anneal to A1. The annealing of A1 to Z2, however, may be displaced by the extension from the 3′ terminus of A1 or 3′ terminus of a nicked product of H2 at the nicking site, and thus will not significantly affect the rate of A2 amplification. Because, in this embodiment, A2 is at least substantially identical to, and optionally is exactly identical to, A1, A2 may also anneal to Region X2 and initiate its own amplification. Such amplification may dramatically increase the rate and level of A2 amplification.

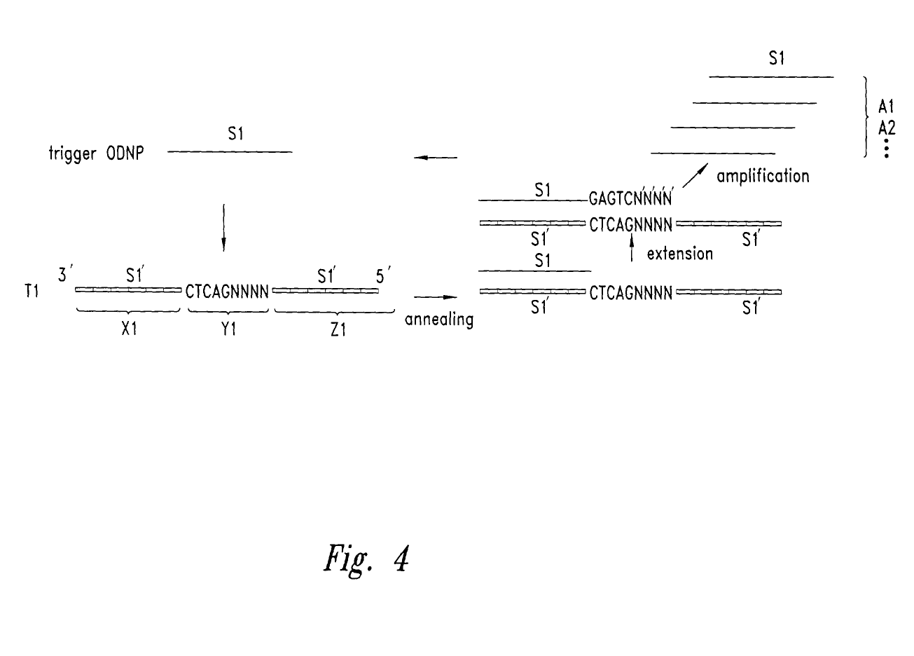

An alternative way of increasing the rate at which a nucleic acid is amplified in a nucleic acid amplification system is to design T1 so that A1 has a sequence identical (or substantially identical) to the sequence of a trigger ODNP for T1. For instance, in an embodiment using the recognition sequence of N.BstNB I as an exemplary recognition sequence shown in FIG. 4, Region X1 and Region Z1 may both comprise an identical sequence (referred to as “S1”) that is substantially or exactly complementary to the sequence of the trigger ODNP (referred to as “S1”). During the first amplification, because A1 is amplified using Region Z1 as a template, A1 has the same sequence as S1. A1 may then function as an oligonucleotide primer for a second amplification reaction using another molecule of T1 as a template. Because the oligonucleotide primer and the template for the first amplification reaction have sequences identical to those of the primer and the template for the second amplification reaction, respectively; the amplified nucleic acid fragment (A2) resulting from the second amplification reaction has the same sequence as that of the amplified nucleic acid fragment (A1) from the first amplification reaction. A2 may then function as an oligonucleotide primer for a third amplification reaction using another molecule of T1 as a template, amplifying a nucleic acid fragment (A3) that is identical to A2. The above process may be repeated multiple times until all T1 molecules anneal to trigger ODNP molecules or amplified fragments (i.e., A1, A2, A3, etc.), or one of the other necessary components of the nucleic acid amplification reactions (e.g., deoxynucleoside triphosphates) is exhausted.

During the above-described nucleic acid amplification process, the presence of a trigger ODNP initiates multiple amplification reactions linked by an amplified nucleic acid fragment from a previous amplification reaction that functions as an amplification primer for a subsequent amplification reaction. Each reaction uses a T1 molecule as a template and amplifies a nucleic acid fragment with a sequence identical to the trigger ODNP. The end result is very rapid amplification of trigger ODNPs in the presence of template T1 molecules. Because this amplification process uses only T1 molecules as templates, it is also referred to as “one-template amplification of a trigger ODNP.”

In some embodiments of one-template amplification of a trigger ODNP, Region X1 may contain an additional sequence other than a sequence (S1x′) that is at least substantially complementary to the sequence of a trigger ODNP(S1). The additional sequence may be between S1x′ and the sequence of the antisense strand of the NARS in T1 and contain no more than 5, 10, 15, 20, 25, 50, or 100 nucleotides. Likewise, Region Z1 may also contain an additional sequence other than a sequence (S1z′) that is at least substantially identical to, and optionally is exactly identical to, S1x′. However, if such an additional sequence is present in Region Z1, S1z′ need be located at the 5′ terminus of T1, unless it is complementary to Region Y1 or a 3′ portion thereof, so that no additional sequence is present at the 3′ terminus of A1 to prevent A1 from being extended using another T1 molecule as a template. In some embodiments, the additional sequence is present between the sequence of the antisense strand of the NARS in T1 and S1z′ and contain no more than 5, 10, 15, 20, 25, 50, or 100 nucleotides.

In related embodiments, a T2 molecule, instead of a T1 molecule, has a sequence located 5′ to the antisense strand of a double-stranded NARS that is at least substantially identical to, and optionally is exactly identical to, a sequence located 3′ to the sequence of the antisense strand of the NARS. Thus, when a single-stranded nucleic acid molecule (A1), which is an amplification product in an amplification reaction, anneals to the T2 molecule, another single-stranded nucleic acid molecule (A2) is exponentially amplified. The A2 is at least substantially identical to, and optionally is exactly identical to, the A1 molecule, and may be exactly identical to the A1 molecule in certain embodiments.

In certain embodiments of one-template amplification of a trigger ODNP, T1 may be at most 50, 75, 100, 150 or 200 nucleotides in length. In some embodiments, S1x′ and/or S1z′ are at least 6, 8, 10, 12, 14, 16, 18, or 20 nucleotides in length. In some preferred embodiments, S1x′ and/or S1z′ are 8 to 24, more preferably, 12 to 17 nucleotides in length.

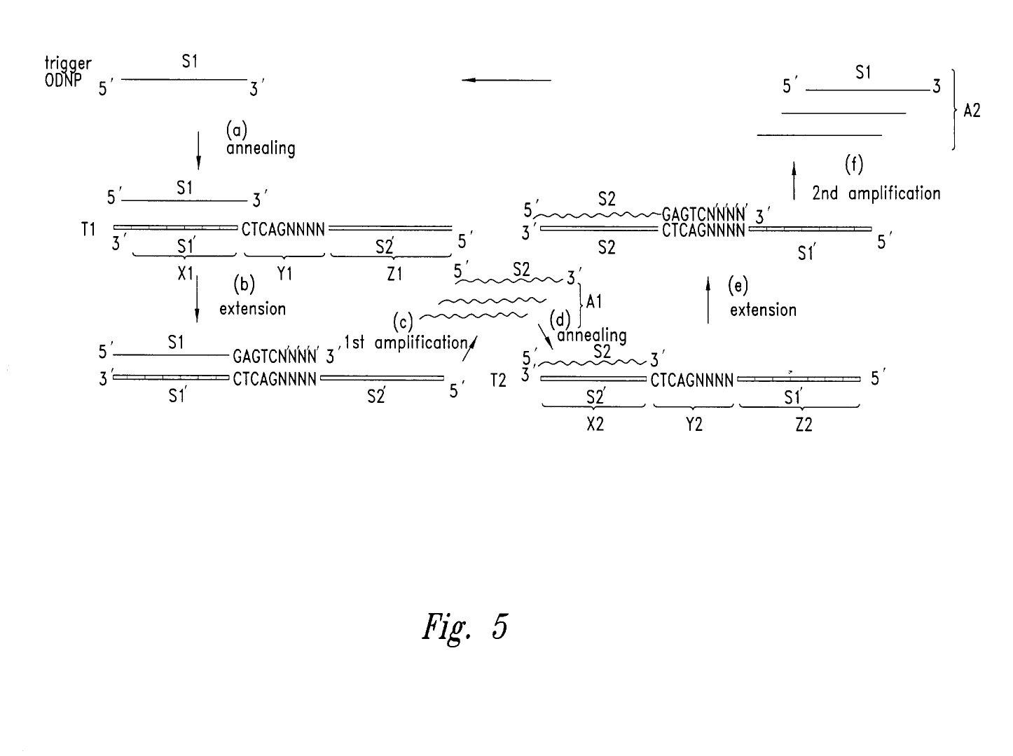

In a related method, a trigger ODNP may be rapidly amplified employing two templates T1 and T2, instead of only one template as in the above-described one-template amplification of a trigger-ODNP. An exemplary embodiment is illustrated in FIG. 5, using the recognition sequence of N.BstNB I as an exemplary NARS, the recognition sequence of which is present in both T1 and T2. As shown in FIG. 5, S1 initiates a first amplification process by annealing to Region X1 of a first template T1 (step (a)), which has a sequence (S1′) complementary to S1. In addition to Region X1, T1 also has Region Y1 directly 5′ to Region X1 with the sequence 3′-CTCAGNNNN-5′ and Region Z1 directly 5′ to Region Y1 with the sequence S2′, which is different from S1′. During the first amplification reaction (step (c)), a nucleic acid fragment A1 having the sequence S2, which is complementary to S2′, is amplified. A1 then anneals to Region X2 of a second template T2 (step (d)). In addition to Region X2, T2 also has Region Y2 directly 5′ to Region X2 with the sequence 3′-CTCAGNNNN-5′ (Each N in Region Y2 may be identical to, or different from, those in the corresponding position of Region Y1) and Region Z2 directly 5′ to Region Y2 with the sequence S1′, which is complementary to S1. During the second amplification reaction (step (f)), a nucleic acid fragment A2 having the sequence S1, which is complementary to S1′ in Region Z2, is amplified. Because the amplified nucleic acid fragment A2 has a sequence identical to the trigger ODNP, it can anneal to T1 and initiate another round of amplification of A1 (i.e., a third amplification reaction). The amplified A1, in turn, initiates another round of amplification of A2 (a fourth amplification reaction). The tandem amplification of A1 and A2 may be repeated multiple times, resulting in rapid accumulation/amplification of the trigger ODNP, which is identical to A2.

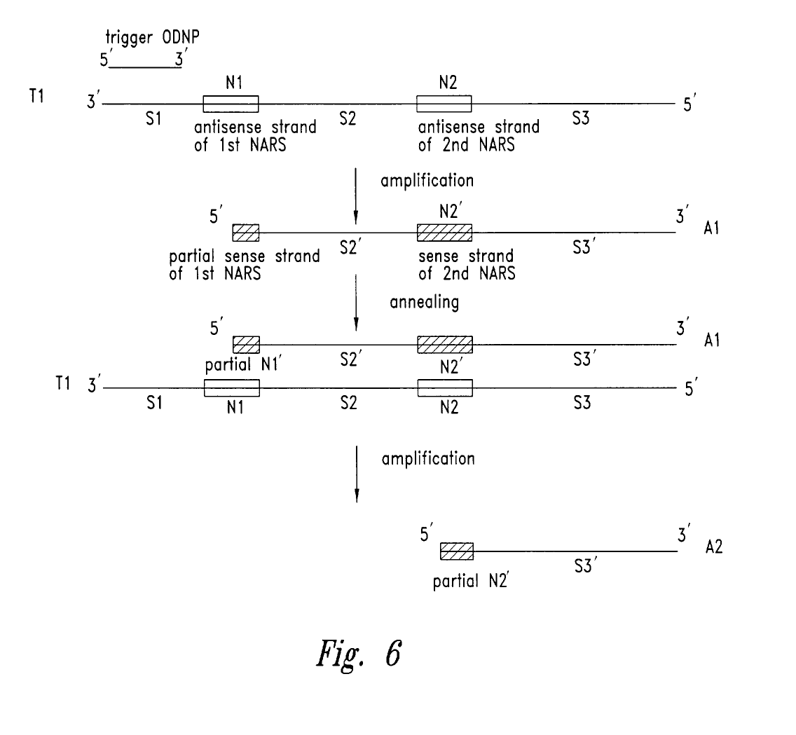

Another embodiment of the first type of nucleic acid amplification (i.e., where both T1 and T2 comprise a sequence of an antisense strand of a NARS) is to use only one template (T1) instead of two templates. Instead of having a single antisense strand of a NARS (“a first NARS”), T1 has an additional antisense strand of another NARS (“a second NARS”). In certain embodiments, the first NARS is different from the second NARS. However, preferably, the first NARS is identical to the second NARS. An exemplary embodiment of this method is illustrated in FIG. 6, where both the first and the second NARSs are recognizable by NAs that nick within their respective recognition sequences. One of ordinary skill in the art will understand that NARSs recognizable by NAs that nick outside their corresponding recognition sequences (e.g., N.BstNB I) may also or alternatively be present in a T1 molecule.

Referring to FIG. 6, T1 contains both the sequence (N1) of the antisense strand of a first NARS and the sequence (N2) of the antisense strand of a second NARS. The sequences 3′ to N1, between N1 and N2, and 5′ to N2 are denoted as sequences S1, S2, and S3, respectively. S1 comprises a sequence that is at least substantially complementary to a trigger ODNP so that the trigger ODNP may anneal to T1 and function as a primer for primer extension in the presence of a DNA polymerase. In the presence of the DNA polymerase and a NA that recognizes the first NARS, a nucleic acid molecule (“A1”) is amplified that comprises, in a 5′ to 3′ direction, a sequence complementary to a partial N1 sequence, a sequence (S2′) complementary to S2, a sequence (N2′) complementary to N2, and a sequence (S3′) complementary to S3. A1 may then anneal to another unoccupied T1 molecule (i.e., a T1 molecule that has not annealed to another molecule, such as a trigger ODNP and an A1 molecule). In the presence of the above DNA polymerase (or another DNA polymerase) and a NA that recognizes the second NARS, a nucleic acid molecule (A2) comprising a sequence complementary to a partial N2 sequence and a sequence complementary to S3 in a 5′ to 3′ direction is amplified.

In certain related embodiments, a T1 molecule may comprise sequences of antisense strands of more than two NARSs. The presence of multiple sequences of antisense strands of NARSs increases the amplification rate of a sequence complementary to the sequence 5′ to the NARS located most closely to the 5′ terminus of the T1 molecule. In various embodiments of the present invention, the T1 molecule may contain no more than 50, 75, 100, 150, or 200 nucleotides. In some embodiments, the shortest distance between two of the multiple sequences of antisense strands of NARSs in T1 is no more than 25, 50, 75, or 100 nucleotides.

In certain other related embodiments, only a T2 molecule (instead of only a T1, or both T1 and T2 molecules) may comprise sequences of antisense strands of two or more NARSs. The presence of multiple sequences of antisense strand of NARSs increases the amplification rate of a sequence complementary to the sequence 5′ to the NARS located most closely to the 5′ terminus of the T2 molecule. In various embodiments, the T2 molecule may contain no more than 50, 75, 100, 150, or 200 nucleotides. In some embodiments, the shortest distance between two of the multiple sequences of antisense strands of NARSs in T2 is no more than 25, 50, 75, or 100 nucleotides.

3. The Second Type of Nucleic Acid Amplification

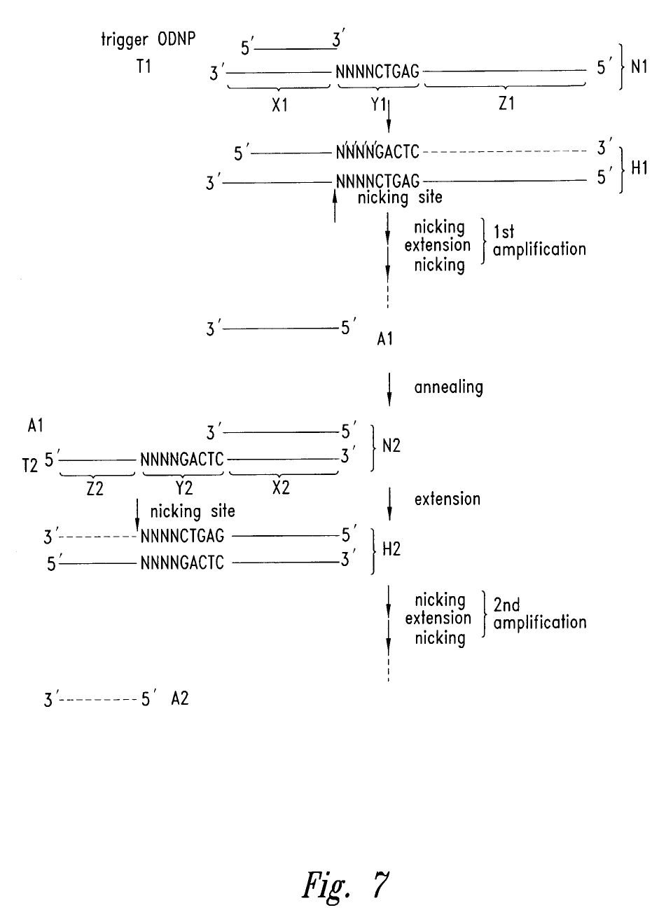

The second type of nucleic acid amplification according to the present invention is where T1 comprises a sequence of a sense strand of a first NARS and T2 comprises a sequence of an antisense strand of a second NARS. Preferably, the first NARS is identical to the second NARS. Using N.BstNB I as an exemplary NA of which the sequence of the sense strand is present in T1 and of which the sequence of the antisense strand is present in T2, this type of nucleic acid amplification is illustrated in FIG. 7. However, one of ordinary skill in the art appreciates that other nicking agents, such as nicking endonucleases other than N.BstNB I, may alternatively be used.

In the exemplary embodiment shown in FIG. 7, the initial nucleic acid molecule N1 is a partially double-stranded nucleic acid molecule formed by annealing a trigger ODNP with T1 having three regions: Regions X1, Y1 and Z1. Regions X1, Y1 and Z1 are defined as the region directly 3′ to the nicking site of the extension product of N1 (i.e., H1) by N.BstNB I, the region from the nicking site to the 5′ terminus of the sequence of the sense strand of the recognition sequence of N.BstNB I (i.e., 5′-GAGTCNNNN-3′ where N can be A, T, G or C), and the region directly 5′ to Region Y2, respectively. The trigger ODNP is complementary to Region X1 or a portion thereof and functions as a primer for nucleic acid extension in the presence of a DNA polymerase. The extension produces a double-stranded nucleic acid fragment (H1) or a partially double-stranded nucleic acid fragment (H1), depending on whether the 5′ terminal sequence of the trigger ODNP anneals to the 3′ terminal sequence of Region X1. The resulting H1 comprises the double-stranded N.BstNB I recognition sequence, which can be nicked by N.BstNB I. In a preferred embodiment, the 3′ terminus of T1 is blocked by a phosphate group so that the extension from this terminus is prevented. The nicked product comprising the sequence of the sense strand of the recognition sequence of N.BstNB I may be extended again from its 3′ terminus at the nicking site by the DNA polymerase, displacing the strand A1 containing the 5′ terminus produced by N.BstNB I at the nicking site. The nicking-extension cycle is repeated multiple times, resulting in the accumulation of the displaced strand A1. A1 is then annealed to Region X2 of T2, (which contains two additional regions, i.e., Regions Y2 and Z2), to form an initial nucleic acid molecule N2 for the second amplification reaction. Region Y2 has the sequence of the antisense strand of the recognition sequence of N.BstNB I and four nucleotides located directly 5′ to the sequence of the antisense strand of the N.BstNB I recognition sequence (i.e., 5′-NNNNGACTC-3′ wherein N can be A, T, G or C). Regions X2 and Z2 refer to regions immediately next to the 3′ terminus and the 5′ terminus of Region Y2, respectively. The extension of A1 using T2 as a template produces a double-stranded nucleic acid molecule (H2) or a partially double-stranded nucleic acid molecule (H2), depending on whether the 5′ terminal sequence of A1 anneals to the 3′ terminal sequence of Region X2. The resulting H2 comprises the double-stranded N.BstNB I recognition sequence, which can be nicked by N.BstNB I. The 3′ terminus at the nicking site may be extended again by the DNA polymerase, displacing the strand A2 containing the 5′ terminus at the nicking site. The nicking-extension cycle is repeated multiple times, resulting in accumulation/amplification of the displaced strand A2. The amplification of A2 is exponential because it is the final amplification product of two linked linear amplification reactions.

Similar to the first type of nucleic acid amplification, A2 may also be designed to have an at least substantially identical sequence to, or a different sequence from, A1 by designing Region Z2 to have a sequence that is at least substantially complementary to A1 or a sequence that is not substantially complementary to A1. When Region Z2 is at least substantially complementary to A1, because A2 is amplified using Region Z2 as a template, A2 is at least substantially identical to, and optionally is exactly identical to, A1. Thus, A2, like A1, may also anneal to Region X2 and initiate its own amplification. Such amplification may greatly increase the rate and level of A2 amplification.

4. Nucleic Acids, Compositions or Kits for Nucleic Acid Amplification

In one aspect, the present invention provides compositions and kits for exponential amplification of nucleic acids. The compositions generally comprise a combination of at least double-stranded nucleic acid molecules N1 (or H1) and N2 (or H2). For instance, for the first type of nucleic acid amplification, the composition may comprise (1) a first at least partially double-stranded nucleic acid molecule (i.e., N1 or H1) of which one strand comprises a sequence of an antisense strand of a first nicking agent recognition sequence, and (2) a second at least partially double-stranded nucleic acid (N2 or H2) of which one strand comprises, from 5′ to 3′: (i) a sequence of an antisense strand of a second nicking agent recognition sequence, and (ii) a sequence that is at least substantially identical to, and optionally is exactly identical to, a sequence located 5′ to the sequence of the antisense strand of the first nicking agent recognition sequence in the first nucleic acid. For the second type of nucleic acid amplification, the composition may comprise: (1) a first at least partially double-stranded nucleic acid molecule (N1 or H1) of which one strand comprises a sequence of a sense strand of a first nicking agent recognition sequence (NARS), and (2) a second at least partially double-stranded nucleic acid molecule (N2 or H2) of which one strand comprises from 5′ to 3′: (i) a sequence of an antisense strand of a second NARS, and (ii) a sequence that is at least substantially complementary to a sequence located 3′ to the sequence of the sense strand of the first nicking agent recognition sequence in the first nucleic acid.

The kit of the present invention may comprise one of the above compositions. Alternatively, the kit may comprise a combination of single-stranded nucleic acid molecules T1 and T2 designed to function in either the first or the second type of nucleic acid amplification described above. For instance, for the first type of nucleic acid amplification, the composition may comprise T1 that comprises the sequence of an antisense strand of a first NARS and T2 that comprises, from 5′ to 3′: a sequence of an antisense strand of a second NARS and a sequence that is at least substantially identical to, and optionally is exactly identical to, a sequence located 5′ to the sequence of the antisense strand of the first NARS in T1. For the second type of nucleic acid amplification, the composition may comprise T1 that comprises a sequence of a sense strand of a first NARS and T2 that comprises, from 5′ to 3′: a sequence of an antisense strand of a second NARS and a sequence that is at least substantially complementary to a sequence located 3′ to the sequence of the sense strand of the first NARS in T1. Preferably, for both types of nucleic acid amplification, the first NARS is identical to the second NARS.

In addition, the present invention also provides a nucleic acid molecule T1 for one-template amplification of a trigger ODNP. T1 may comprise a sequence of an antisense strand of a NARS, and a nucleotide sequence located both 5′ to and 3′ to the sequence of the antisense strand of the NARS. The above nucleotide sequence is generally at least 8 nucleotides in length, preferably at least 9, 10, 11, 12, 13, 14 nucleotides in length to allow for the specific annealing between the trigger ODNP and the oligonucleotide sequence in T1.

The present invention further provides a composition or kit for two-template amplification of a trigger ODNP or an oligonucleotide substantially identical to the trigger ODNP. Generally, the composition comprises at least two single-stranded nucleic acid molecules T1 and T2. T1 comprises, from 3′ to 5′: an oligonucleotide sequence (S1′), a sequence of an antisense strand of a first NARS and another oligonucleotide sequence (S2′) that is not substantially identical to S1′, whereas T2 comprises, from 3′ to 5′: an oligonucleotide sequence that is at least substantially identical to, and optionally is exactly identical to, S2′, a sequence of an antisense strand of a second NARS, and a sequence that is at least substantially identical to S1′. Generally, both S1′ and S2′ are at least 8 nucleotides in length to allow for specific annealing between the trigger ODNP and S1′ in T1 and between the nucleic acid molecule (A1) amplified using S2′ in T1 as a template and the sequence that is at least substantially identical to S2′ in T2. In certain embodiments, T1 or T2 or both may be at least 9, 10, 11, 12, 13, 14 nucleotides in length. Preferably, the first NARS is identical to the second NARS.

In addition to the above-described nucleic acid molecules, the kits (or compositions) of the present invention may further comprise at least one, two, several, or each of the following components: (1) a trigger ODNP that is capable of specific annealing to the sequence of T1 3′ to the sequence of one strand of the NARS in T1; (2) a nicking agent (e.g., a NE or a RE) that recognizes the NARS of which the sequence of one strand is present in T1, T2 or both; (3) a buffer for nicking agent (2); (4) a DNA polymerase useful for primer extension; (5) a buffer for DNA polymerase (4); (6) deoxynucleoside triphosphates; (7) a modified deoxynucleoside triphosphate; (8) a control T1, T2 and/or trigger ODNP; and (9) a strand displacement facilitator (e.g., trehalose). Detailed descriptions of many of the above components are provided above.

In certain embodiments, the composition of the present invention does not contain a buffer specific to a NA or a buffer specific to a DNA polymerase. Instead, it contains a buffer suitable for both the nicking agent and the DNA polymerase. For instance, if N.BstNB I is the nicking agent and exo⁻ Vent is the DNA polymerase, the nicking-extension buffer can be 0.5×N.BstNB I buffer and 1×exo⁻ Vent Buffer.

The compositions of the present invention may be made by simply mixing their components or by performing reactions that results in the formation of the compositions. The kits of the present invention may be prepared by mixing some of their components or keep each of their components in an individual container.

5. Immobilized Nucleic Acids and Arrays of Nucleic Acids

In certain embodiments, the nucleic acids or oligonucleotides that involve in exponential nucleic acid amplification according to the present invention may be immobilized to a solid support (also referred to as a “substrate”). The nucleic acids or oligonucleotides that may be immobilized include target nucleic acids, oligonucleotide primers useful for preparing an initial nucleic acid (described below), trigger ODNPs, T1 molecules, and T2 molecules. In certain embodiments, such nucleic acids or oligonucleotides may be immobilized via their 5′ or 3′ termini if they are single-stranded, or via their 5′ or 3′ termini of one strand if they are double-stranded.

The methods for immobilizing a nucleic acid or an oligonucleotide are known in the art. In certain embodiments, nucleic acids or oligonucleotides of the present invention are immobilized to a substrate to form an array. As used herein, an “array” refers to a collection of nucleic acids or oligonucleotides that are placed on a solid support in distinct areas. Each area is separated by some distance in which no nucleic acid or oligonucleotide is bound or deposited. In some embodiments, area sizes are 20 to 500 microns and the center to center distances of neighboring areas range from 50 to 1500 microns. The array of the present invention may contain 2-9, 10-100, 101-400, 401-1,000, or more than 1,000 distinct areas.

Generally, the nucleic acid or oligonucleotide may be immobilized to a substrate in the following two ways: (1) synthesizing the nucleic acids or the oligonucleotides directly on the substrate (often termed “in situ synthesis”), or (2) synthesizing or otherwise preparing the nucleic acid or the oligonucleotides separately and then position and bind them to the substrate (sometimes termed “post-synthetic attachment”). For in situ synthesis, the primary technology is photolithography. Briefly, the technology involves modifying the surface of a solid support with photolabile groups that protect, for example, oxygen atoms bound to the substrate through linking elements. This array of protected hydroxyl groups is illuminated through a photolithographic mask, producing reactive hydroxyl groups in the illuminated areas. A 3′-O-phosphoramidite-activated deoxynucleoside protected at the 5′-hydroxyl with the same photolabile group is then presented to the surface and coupling occurs through the hydroxyl group at illuminated areas. Following further chemical reactions, the substrate is rinsed and its surface is illuminated through a second mask to expose additional hydroxyl groups for coupling. A second 5′-protected, 3′-O-phosphoramidite-activated deoxynucleoside is present to the surface. The selective photo-de-protection and coupling cycles are repeated until the desired set of products is obtained. Detailed description of using photolithography in array fabrication may be found in the following patents or published patent applications: U.S. Pat. Nos. 5,143,854; 5,424,186; 5,856,101; 5,593,839; 5,908,926; 5,737,257; and Published PCT Patent Application Nos. WO99/40105; WO99/60156; WO00/35931.

The post-synthetic attachment approach requires a methodology for attaching pre-existing oligonucleotides to a substrate. One method uses the biotin-streptavidin interaction. Briefly, it is well known that biotin and streptavidin form a non-covalent, but very strong, interaction that may be considered equivalent in strength to a covalent bond. Alternatively, one may covalently bind pre-synthesized or pre-prepared nucleic acids or oligonucleotides to a substrate. For example, carbodiimides are commonly used in three different approaches to couple DNA to solid supports. In one approach, the support is coated with hydrazide groups that are then treated with carbodiimide and carboxy-modified oligonucleotide. Alternatively, a substrate with multiple carboxylic acid groups may be treated with an amino-modified oligonucleotide and carbodiimide. Epoxide-based chemistries are also used with amine modified oligonucleotides. Detailed descriptions of methods for attaching pre-existing oligonucleotides to a substrate may be found in the following references: U.S. Pat. Nos. 6,030,782; 5,760,130; 5,919,626; published PCT Patent Application No. WO00/40593; Stimpson et al. Proc. Natl. Acad. Sci. 92:6379-6383 (1995); Beattie et al. Clin. Chem. 41:700-706 (1995); Lamture et al. Nucleic Acids Res. 22:2121-2125 (1994); Chrisey et al. Nucleic Acids Res. 24:3031-3039 (1996); and Holmstrom et al., Anal. Biochem. 209:278-283 (1993).

The primary post-synthetic attachment technologies include ink jetting and mechanical spotting. Ink jetting involves the dispensing of nucleic acids or oligonucleotides using a dispenser derived from the ink-jet printing industry. The nucleic acid oligonucleotides are withdrawn from the source plate up into the print head and then moved to a location above the substrate. The nucleic acids or oligonucleotides are then forced through a small orifice, causing the ejection of a droplet from the print head onto the surface of the substrate. Detailed description of using ink jetting in array fabrication may be found in the following patents: U.S. Pat. Nos. 5,700,637; 6,054,270; 5,658,802; 5,958,342; 6,136,962 and 6,001,309.

Mechanical spotting involves the use of rigid pins. The pins are dipped into a nucleic acid or oligonucleotide solution, thereby transferring a small volume of the solution onto the tip of the pins. Touching the pin tips onto the substrate leaves spots, the diameters of which are determined by the surface energies of the pins, the nucleic acid or oligonucleotide solution, and the substrate. Mechanical spotting may be used to spot multiple arrays with a single nucleic acid or oligonucleotide loading. Detailed description of using mechanical spotting in array fabrication may be found in the following patents or published patent applications: U.S. Pat. Nos. 6,054,270; 6,040,193; 5,429,807; 5,807,522; 6,110,426; 6,063,339; 6,101,946; and published PCT Patent Application Nos. WO99/36760; 99/05308; 00/01859; 00/01798.

One of ordinary skill in the art would appreciate that besides the techniques described above, other methods may also be used in immobilizing nucleic acids or oligonucleotides to a substrate. Descriptions of such methods can be found in, but are not limited to, the following patent or published patent applications: U.S. Pat. Nos. 5,677,195; 6,030,782; 5,760,130; and 5,919,626; and published PCT Patent Application Nos. WO98/01221; WO99/41007; WO99/42813; WO99/43688; WO99/63385; WO00/40593; WO99/19341; and WO00/07022.

The substrate to which the nucleic acids or oligonucleotides of the present invention are immobilized to form an array is prepared from a suitable material. The substrate is preferably rigid and has a surface that is substantially flat. In some embodiments, the surface may have raised portions to delineate areas. Such delineation separates the amplification reaction mixtures at distinct areas from each other and allows for the amplification products at distinct areas to be analyzed or characterized individually. The suitable material includes, but is not limited to, silicon, glass, paper, ceramic, metal, metalloid, and plastics. Typical substrates are silicon wafers and borosilicate slides (e.g., microscope glass slides). An example of a particularly useful solid support is a silicon wafer that is usually used in the electronic industry in the construction of semiconductors. The wafers are highly polished and reflective on one side and can be easily coated with various linkers, such as poly(ethyleneimine) using silane chemistry. Wafers are commercially available from companies such as WaferNet, San Jose, Calif.

Depending on the contemplated application, one of ordinary skill in the art may vary the composition of immobilized molecules of the present array. For instance, the T1 or T2 molecules of the present invention may or may not be immobilized to every distinct area of the array. Preferably, the nucleic acids or oligonucleotides in a distinct area of an array are homogeneous. More preferably, the nucleic acids or oligonucleotides in every distinct area of an array to which the nucleic acids or oligonucleotides are immobilized are homogeneous. The term “homogeneous,” as used herein, indicates that each nucleic acid or oligonucleotide molecule in a distinct area has the same sequence as another nucleic acid or oligonucleotide molecule in the same area. Alternatively, the nucleic acid or oligonucleotide in at least one of the distinct areas of an array are heterogeneous. The term “heterogeneous,” as used herein, indicates that at least one nucleic acid or oligonucleotide molecule in a distinct area has a different sequence from another nucleic acid or oligonucleotide molecule in the area. In some embodiments, molecules other than the nucleic acids or oligonucleotides described above may also be present in some or all of distinct areas of an array. For instance, a molecule useful as an internal control for the quality of an array may be attached to some or all of distinct areas of an array. Another example for such a molecule may be a nucleic acid useful as an indicator of hybridization stringency. In other embodiments, the composition of nucleic acids or oligonucleotides in every distinct area of an array is the same. Such an array may be useful in determining genetic variations in a particular gene in a selected population of organisms or in parallel diagnosis of a disease or a disorder associated with mutations in a particular gene.

Depending on the envisioned application, the immobilized nucleic acids or oligonucleotides of the present invention (e.g., the T1 or T2 molecules) may contain oligonucleotide sequences that are at least substantially complementary or identical to various target nucleic acids. Such target nucleic acids include, but are not limited to, genes associated with hereditary diseases in animals, oncogenes, genes related to disease predisposition, genomic DNAs useful for forensics and/or paternity determination, genes associated with or rendering desirable features in plants or animals, and genomic or episomic DNA of infectious organisms. An array of the present invention may contain nucleic acids or oligonucleotides that are at least substantially complementary or identical to a particular type of target nucleic acids in distinct areas. For example, an array may have a nucleic acid or an oligonucleotide that is at least substantially complementary or identical to a first gene related to disease predisposition in a first distinct area, another nucleic acid or an oligonucleotide that is at least substantially complementary or identical to a second gene also related to disease predisposition in a second distinct area, yet another nucleic acid or an oligonucleotide that is at least substantially complementary or identical to a third gene also related to disease predisposition in a third distinct area, etc. Such an array is useful to determine disease predisposition of an individual animal (including a human) or a plant. Alternatively, an array may have nucleic acids or oligonucleotides that are at least substantially complementary or identical to multiple types of target nucleic acids categorized by the functions of the targets.

In addition, an array may contain nucleic acids or oligonucleotides that are at least substantially complementary or identical to a portion of a target nucleic acid that contains various potential genetic variations. For instance, a first area of the array may contain immobilized nucleic acids or oligonucleotides that are at least substantially complementary or identical to a portion of a target gene that contains a genetic variation of one allele of the target. A second area of the array may contain immobilized nucleic acids or oligonucleotides that are at least substantially complementary or identical to a portion of target gene that contains a genetic variation of another allele of the target. The array may have additional areas that contain immobilized nucleic acids or oligonucleotides that are at least substantially complementary or identical to portions of the target gene that contains genetic variations of additional alleles of the target.

In general, for successful performance in an array environment, the immobilized nucleic acids or oligonucleotides must be stable and not dissociate during various treatment, such as hybridization, washing or incubation at the temperature at which an amplification reaction is performed. The density of the immobilized nucleic acids or oligonucleotides must be sufficient for the subsequent analysis. For an array suitable for the present methods, typically 1000 to 10¹², preferably 1000 to 10^{6, 10}⁶ to 10⁹, or 10⁹ to 10¹² ODNP molecules are immobilized in at least one distinct area. However, there must be minimal non-specific binding of other nucleic acids to the substrate. The immobilization process should not interfere with the ability of immobilized nucleic acids or oligonucleotides required for exponential nucleic acid amplification.

In certain embodiments, it may be desirable to have the nucleic acids or oligonucleotides of the present invention indirectly bound to the substrate via a linker. The linker (also referred to as a “linking element”) comprises a chemical chain that serves to distance the nucleic acids or oligonucleotides from the substrate. In certain embodiments, the linker may be cleavable. There are a number of ways to position a linking element. In one common approach, the substrate is coated with a polymeric layer that provides linking elements with a lot of reactive ends/sites. A common example is glass slides coated with polylysine, which are commercially available. Another example is substrates coated with poly(ethyleneimine) as described in Published PCT Application No. WO99/04896 and U.S. Pat. No. 6,150,103.

The array of the present invention enables the high throughput of various analyses to which the present nucleic acid amplification is applicable. For instance, an array of T2 molecules may be used to amplify multiple target nucleic acids. The reaction mixture or the products of an amplification reaction performed in the presence of a target nucleic acid may be pooled together and applied to the array of T2 molecules. Alternatively, the reaction mixtures or the amplification products of different amplification reactions may be applied to distinct areas of the array. Another round (“second round”) of amplification reactions may then be performed on the array in the present of a nicking agent that recognizes the nicking agent recognition sequence of which the antisense strand is present in the T2 molecules. The amplification products of the second round of reactions performed on the array may be pooled together and analyzed. If the array (e.g., a microwell array) has distinct areas that are delineated by certain physical barriers, the amplification products of the second round of reactions in distinct arrays may be analyzed individually.

For the nucleic acid molecules of the present invention that do not form an array, they may be immobilized via the methods described above that are useful in preparing an array. In addition, any methods known in the art may be used. For instance, a target nucleic acid of the present invention may be immobilized by the use of a fixative or tissue printing. It may also be first isolated or purified and then transferred to a substrate that binds to nucleic acids or oligonucleotides, such as nitrocellulose or nylon membranes.

C. Diagnostic Uses of Nucleic Acid Amplification Methods and Compositions

As described in detail herein above, the present invention provides methods and compositions for exponential amplification of nucleic acids. These methods and compositions may find utility in a wide variety of applications where it is desirable to rapidly amplify a nucleic acid molecule. Such rapid amplification may be especially desirable in diagnostic applications, such as where it is desirable to quickly detect the presence of a pathogen (e.g., bacteria, viruses, fungi, parasites) in a biological sample. The following sections describe various exemplary embodiments specifically applicable for diagnostic uses; however, such embodiments may also be useful in other applications.

1. Overview

The present invention is useful for detecting a target nucleic acid molecule in a biological sample. The target nucleic acid includes a nucleic acid molecule that is derived or originates from a pathogenic organism. Depending on the presence or absence of the target nucleic acid in the sample, an amplification product may or may not be detected in an amplification system that is designed to use the target nucleic acid or its portion as a template. The target nucleic acid or its portion is first incorporated into an initial nucleic acid molecule (N1) to be used as a template in a first amplification reaction. The initial nucleic acid molecule also comprises at least one strand of a first NARS and thus triggers the first amplification reaction in the presence of a DNA polymerase and a NA that recognizes the first NARS. The product (A1) from the first amplification reaction then anneals to another template nucleic acid molecule (T2). T2 comprises a sequence of the antisense strand of a second NARS and thus initiate a second amplification reaction in the presence of the DNA polymerase and a NA that recognizes the second NARS. The determination of the presence or absence of the product (A1) of the first amplification reaction and/or the product (A2) of the second amplification reaction indicates the presence or absence of the target nucleic acid in the biological sample.

2. Initial Nucleic Acid Molecule (N1)

Initial nucleic acid molecules useful for diagnostic applications may be provided by various approaches. For instance, N1 may be obtained by annealing of a trigger ODNP to a T1 molecule where the trigger ODNP is derived from a nucleic acid molecule originated from a pathogenic organism. Alternatively, N1 may be directly derived from a double-stranded nucleic acid molecule originated from a pathogenic organism. N1 may also be a partially double-stranded nucleic acid molecule having an overhang derived from a target nucleic acid and functioning as a template for single-stranded nucleic acid amplification, or an overhang capable of hybridizing with a target nucleic acid but not functioning as a template for single-stranded nucleic acid amplification. These and other means for providing N1 relevant to diagnostic applications are described below.

a. First Type of Exemplary Methods for Providing N1 Molecules

In certain embodiments of the present invention where N1 is provided by annealing a trigger ODNP to a T1 molecule, the trigger ODNP may be derived from either a DNA molecule (e.g., a genomic DNA molecule) or a RNA molecule (e.g., a mRNA molecule) originated from a pathogenic organism. If the nucleic acid molecule originated from a pathogenic organism is single-stranded, it may be directly used as a trigger ODNP. Alternatively, the single-stranded nucleic acid may be cleaved to produce shorter fragments, where one or more of these fragments may to be used as a trigger ODNP. If the nucleic acid molecule originated from a pathogenic organism is double-stranded, it may be denatured and directly used as a trigger ODNP or the denatured product may be cleaved to provide multiple shorter single-stranded fragments where one or more of these fragments may function as an ODNP trigger. Alternatively, it may be first cleaved to obtain multiple shorter double-stranded fragments, and the shorter fragments are then denatured to provide one or more trigger ODNPs.

As discussed above, a T1 molecule must be at least substantially complementary to the trigger ODNP. In addition, the number of T1 molecules in an amplification reaction mixture is preferably greater than that of the trigger ODNP to effectively compete with the complementary strand of the trigger ODNP originated from the double-stranded nucleic acid molecule for annealing to the trigger ODNP.

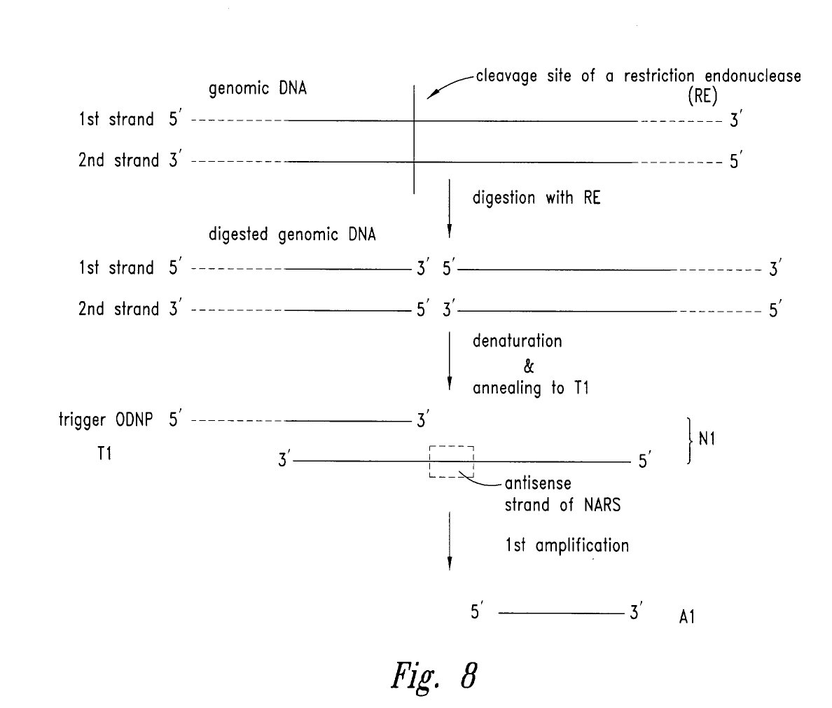

An example of the first type of methods for preparing N1 molecules is shown in FIG. 8. As indicated in this figure, a double-stranded genomic DNA may be first cleaved by a restriction endonuclease. The digestion products may be denatured and one strand of one of the digestion products may be used as a trigger ODNP to initiate nucleic acid amplification reactions.

In certain preferred embodiment, T1 may comprise, from 3′ to 5′: a first sequence that is at least substantially identical to the trigger ODNP, a sequence of the antisense strand of a nicking agent recognition sequence, and a second sequence that is at least substantially identical to the first sequence. Such a T1 molecule allows exponential amplification of a trigger ODNP or a nucleic acid fragment that is substantially identical to the trigger ODNP without any additional template nucleic acid molecules.

b. Second Type of Exemplary Methods for Providing N1 Molecules

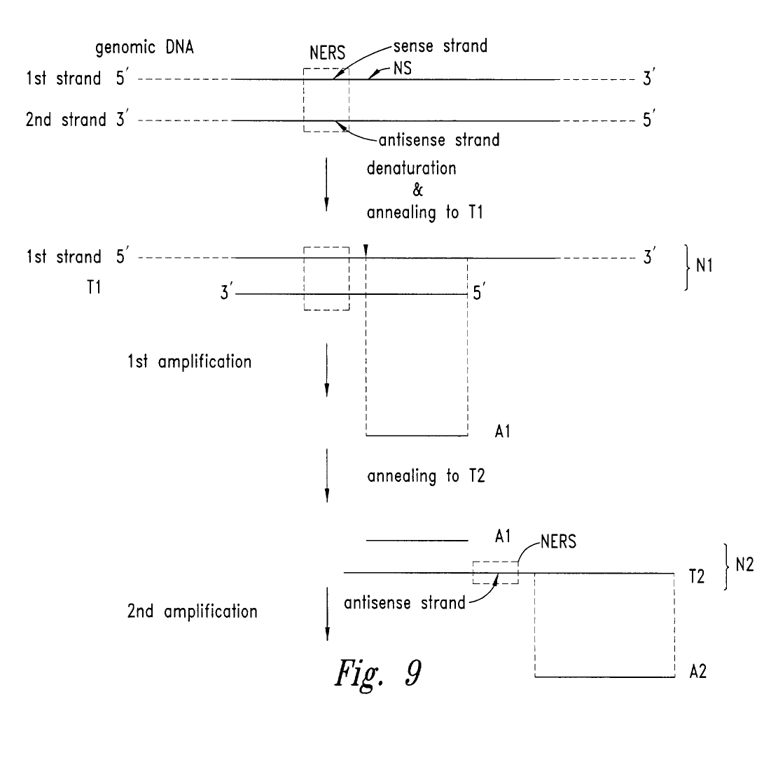

In certain embodiments of the present invention where N1 is provided by annealing a trigger ODNP to a T1 molecule, the trigger ODNP comprises the sequence of the sense strand of a NARS. The trigger ODNP may be derived from a target nucleic acid (e.g., a genomic nucleic acid) originated from a pathogenic organism. A specific embodiment where N1 comprises a NERS recognizable by a nicking endonuclease that nicks outside its recognition sequence (e.g. N.BstNB I) is illustrated in FIG. 9. As illustrated by this figure, a genomic DNA or a fragment thereof comprising a NERS is denatured and one strand of the genomic DNA or a fragment of that strand anneals to a T1 molecule. The T1 molecule is a portion of the other strand of the genomic DNA that comprises a sequence of the antisense strand of the NERS. The annealing of the trigger ODNP to the T1 molecule provides the initial nucleic acid molecule N1 for amplification reactions. The number of T1 molecules in an amplification reaction mixture is preferably greater than the number of strands of genomic DNA or fragments thereof that contain the sequence of the sense strand of the NERS.

In related embodiments where the trigger ODNP is derived from a target nucleic acid and comprises the sequence of the sense strand of a NARS, a T1 molecule may be at least substantially complementary to the trigger ODNP at its 3′ portion, but not at its 5′ portion. The 3′ portion of T1 includes the sequence of the antisense strand of the NARS so that the initial nucleic acid formed by annealing T1 to the trigger ODNP comprises a double-stranded NARS. In the presence of a NA that recognizes the NARS, the N1 molecule is nicked. The 3′ terminus at the nicking site is then extended using a region 5′ to the sequence of the antisense strand of the NARS in the T1 molecule as the template. The resulting amplification product is a single-stranded nucleic acid molecule that is complementary to a region of T1 located 5′ to the sequence of the antisense strand of the NARS rather than a portion of the trigger ODNP.

c. Third Type of Exemplary Methods for Providing N1 Molecules

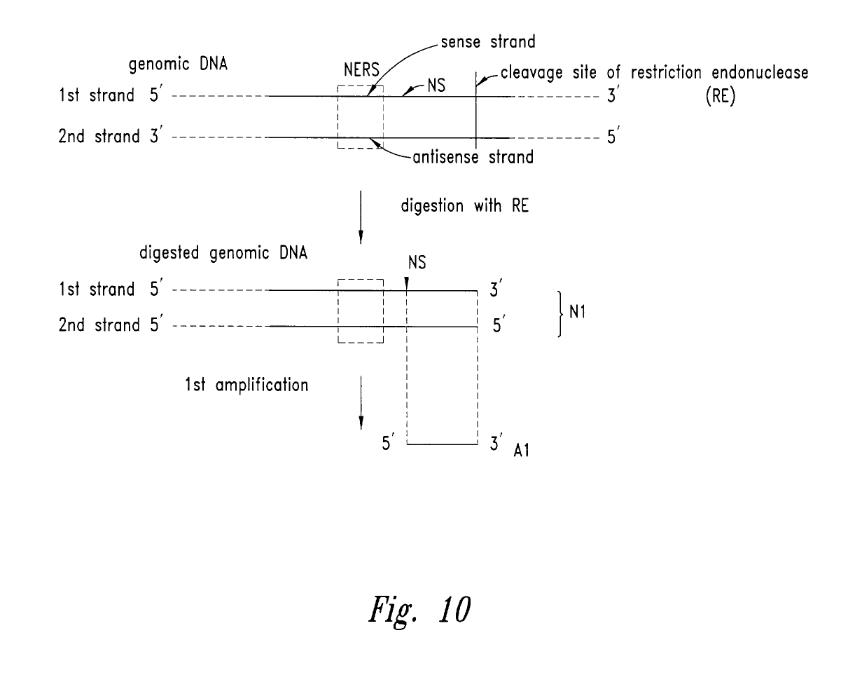

In certain embodiments of the present invention, N1 is a double-stranded nucleic acid derived directly from a genomic nucleic acid that contain both a NARS and a RERS, where the NS corresponding to the NARS lies between the NARS and the RERS, and the RERS is located near the NARS. An embodiment with a NERS recognizable by a nicking endonuclease that nicks outside its recognition sequence (e.g., N.BstNB I) as an exemplary NARS is illustrated in FIG. 10. As shown in this figure, genomic DNA may be digested by a restriction endonuclease that recognizes a RERS in the genomic DNA. The digestion product that contains the NERS may function as an initial nucleic acid molecule (N1). When a NE recognizes the NERS in the N1, and nicks N1 at the NS a short fragment (A1) is produced that functions as an at least initial amplification primer in a subsequent amplification reaction. Multiple copies of A1 are created upon repetitive nicking at and extending from the NS. A1 is only produced if genomic DNA of a certain quality is present in the sample.

d. Fourth Type of Exemplary Methods for Providing N1 Molecules

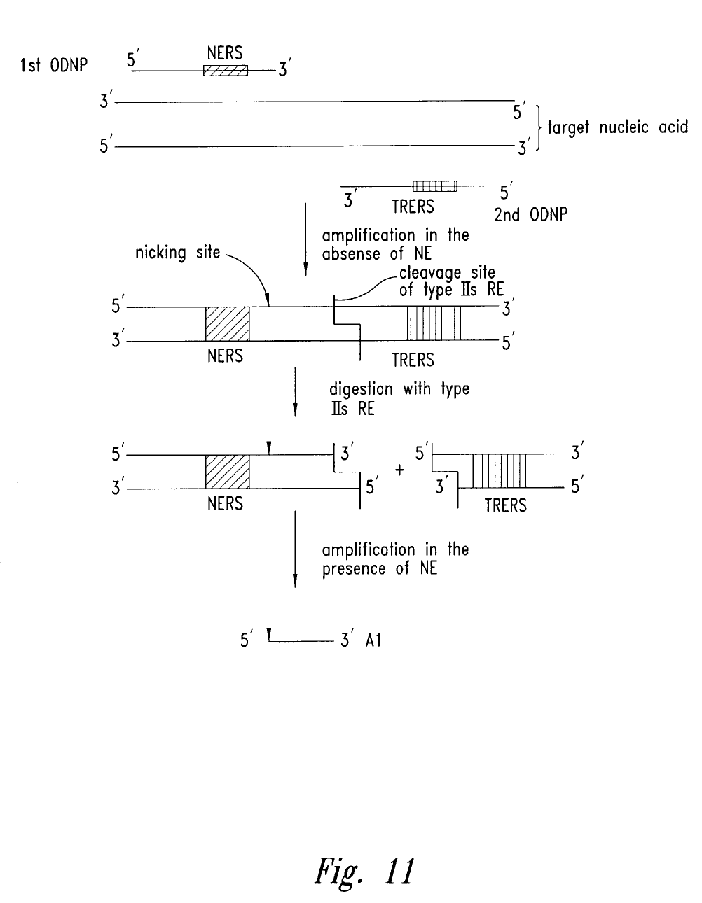

In certain embodiments of the present invention, an initial nucleic acid molecule N1 is a completely or partially double-stranded nucleic acid molecule produced using various ODNP pairs. The methods for using ODNP pairs to prepare N1 molecules are described below in connection with FIGS. 11-13.

In one embodiment, a precursor to N1 contains a double-stranded NARS and a RERS. The NARS and RERS are incorporated into the precursor using an ODNP pair. An embodiment with a NERS recognizable by a NE that nicks outside its recognition sequence (e.g., N.BstNB I) as an exemplary NARS, and a type IIs restriction endonuclease recognition sequence (TRERS) as an exemplary RERS is illustrated in FIG. 11. As shown in this figure, a first ODNP comprises the sequence of one strand of a NERS while a second ODNP comprises the sequence of one strand of a TRERS. When these two ODNPs are used as primers to amplify a portion of a target nucleic acid, the resulting amplification product (i.e., a precursor to N1), contains both a double-stranded NERS and a double-stranded TRERS. In the presence of a type IIs restriction endonuclease that recognizes the TRERS, the amplification product is digested to produce a nucleic acid molecule N1 that comprises a double-stranded NERS.

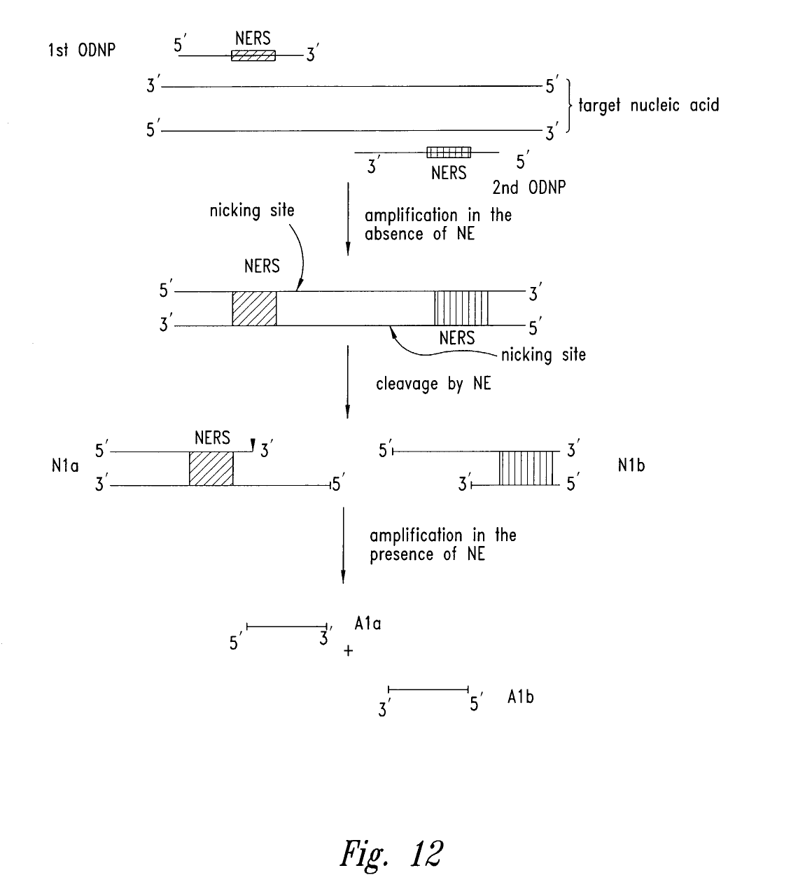

In another embodiment, a precursor to N1 contains two double-stranded NARSs. The two NARSs are incorporated into the precursor to N1 using two ODNPs. An embodiment with a NERS recognizable by a nicking endonuclease that nicks outside its recognition sequence as an exemplary NARS is illustrated in FIG. 12. As shown in this figure, both ODNPs comprise a sequence of a sense strand of a NERS. When these two ODNPs are used as primers to amplify a portion of a target nucleic acid, the resulting amplification product contains two NERSs. These two NERSs may or may not be identical to each other, but preferably, they are identical. In the presence of a NE or NEs that recognize the NERSs, the amplification product is nicked twice (once on each strand) to produce two nucleic acid molecules (N1 a and N1b) that each comprises a double-stranded NERS.

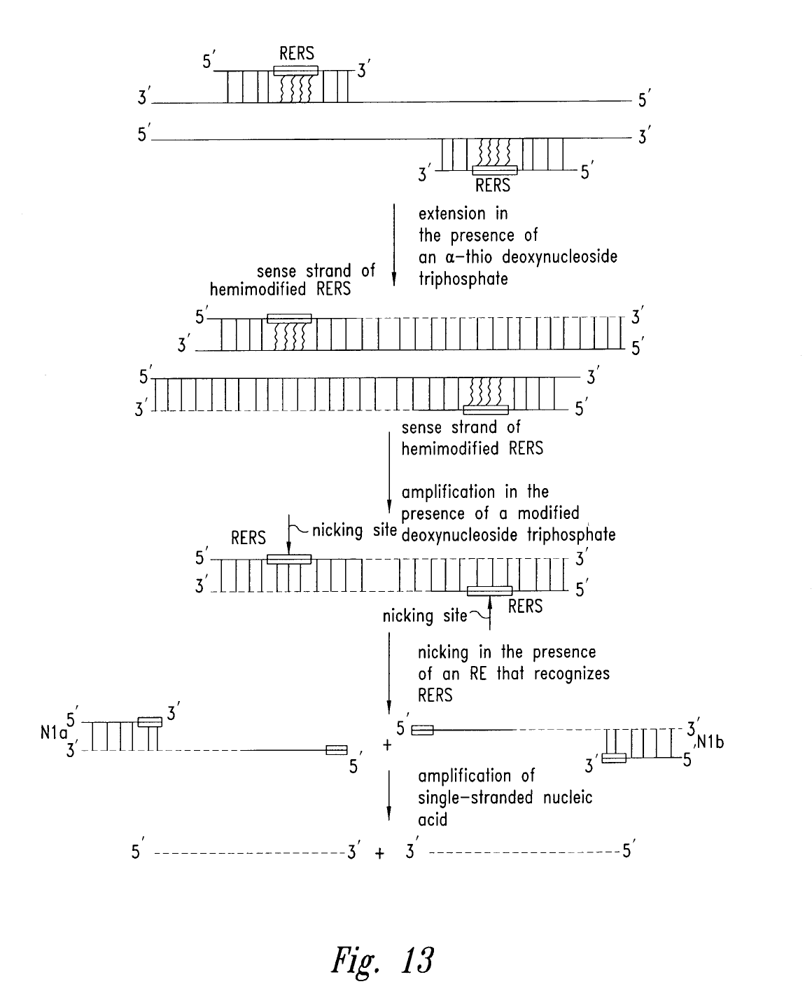

In yet another embodiment, a precursor to N1 contains two hemimodified RERS. The two hemimodified RERSs are incorporated into the precursor by the use of two ODNPs. This embodiment is illustrated in FIG. 13. As shown in this figure, both the first and the second ODNPs comprise a sequence of one strand of a RERS. When these two ODNPs are used as primers to amplify a portion of a target nucleic acid in the presence of a modified deoxynucleoside triphosphate, the resulting amplification product contains two hemimodified RERSs. These two hemimodified RERS may or may not be identical to each other. In the presence of a RE or REs that recognize the hemimodified RERS, the above amplification product is nicked to produce two partially double-stranded nucleic acid molecule (N1a and N1b) that each comprises a sequence of at least one strand of the hemimodified RERS.

e. Fifth Type of Exemplary Methods for Providing N1 Molecules

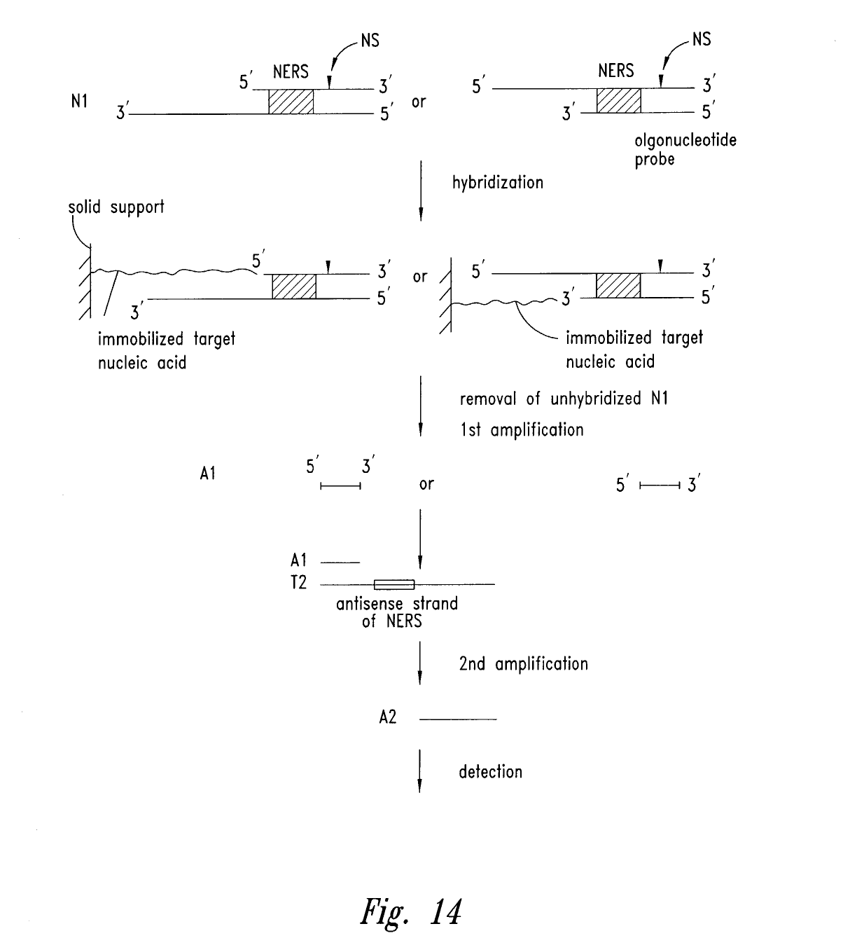

In other embodiments of the present invention, an initial nucleic acid molecule N1 is a partially double-stranded nucleic acid molecule having a NARS and an overhang at least substantially complementary to a target nucleic acid, but not functioning as a template for single-stranded nucleic acid amplification. An exemplary embodiment wherein N1 has a NERS recognizable by a nicking endonuclease that nicks outside its recognition sequence as an exemplary NARS is illustrated in FIG. 14. As shown in this figure, the N1 molecule may contain a 5′ overhang in the strand that either comprises a NS or forms a NS upon extension. Alternatively, the N1 molecule may contain a 3′ overhang in the strand that either comprises a NS or forms a NS upon extension. The overhang of the N1 molecule must be at least substantially complementary to a target nucleic acid molecule so that it can anneal to the target nucleic acid molecule, if present, in a biological sample of interest. The N1 molecules that do not anneal to the target nucleic acid are then removed. The remaining N1 molecules that anneal to the target nucleic acid (if present in the sample) is used to amplify a single-stranded nucleic acid molecule A1.

The removal of N1 molecules that do not anneal to the target nucleic acid may be facilitated by immobilizing the nucleic acid molecules in the biological sample to a solid support as shown in FIG. 14. Such immobilization may be performed by any method known in the art, including without limitation, the use of a fixative or tissue printing. A N1 molecule having an overhang that is substantially complementary to a particular target nucleic acid molecule is then applied to the sample. If the target nucleic acid is present in the sample, N1 hybridizes to the target nucleic acid via its overhang. The sample is subsequently washed to remove any unhybridized N1 molecule. In the presence of a DNA polymerase and nicking endonuclease that recognizes the NERS in N1, a single-stranded nucleic acid molecule A1 is amplified. In the further presence of a suitable T2 molecule, another single-stranded nucleic acid molecule A2 is amplified. However, if the target nucleic acid is absent in the sample, N1 is unable to hybridize to any nucleic acid molecule in the sample and thus is washed off from the sample. Thus, when the washed biological sample is incubated with nucleic acid amplification reaction mixture (i.e., a mixture containing all the necessary components for single strand nucleic acid amplification using a portion of N1 as a template, such as a NE that recognizes the NERS in the N1 molecule and a DNA polymerase), no single-stranded nucleic acid molecule that is complementary to the above portion of N1 is amplified.

Besides immobilizing a target nucleic acid molecule, a target-N1 complex may be purified by first hybridizing the N1 molecule with the target nucleic acid molecule in a biological sample and then isolating the complex by a functional group associated with the target nucleic acid. For instance, the target nucleic acid may be labeled with a biotin molecule, and the target-N1 complex may be subsequently purified via the biotin molecule associated with the target, such as precipitating the complex with immobilized streptavidin.

In certain related embodiments, N1 is formed by hybridizing an immobilized target nucleic acid from a biological sample with a single-stranded T1 molecule. An example of these embodiments is where a target nucleic acid is not immobilized, but a T1 molecule as described above is immobilized to a solid support via its 5′ terminus. If a target nucleic acid is present in a sample, the hybridization of the nucleic acids of the sample to the T1 allows the target to remain attached to the solid support when the solid support is washed. In the presence of a nicking agent that recognizes the nicking agent recognition sequence of which the antisense strand is present in the T1 and a DNA polymerase, a single-stranded nucleic acid molecule is amplified using a sequence located 5′ to the sequence of the antisense strand of the recognition sequence in the T1 as a template. If the target is absent in the sample, the nucleic acids of the sample will be washed off the solid support to which the T1 is attached. Thus, no single-stranded nucleic acid molecule is amplified using a portion of the T1 as a template.

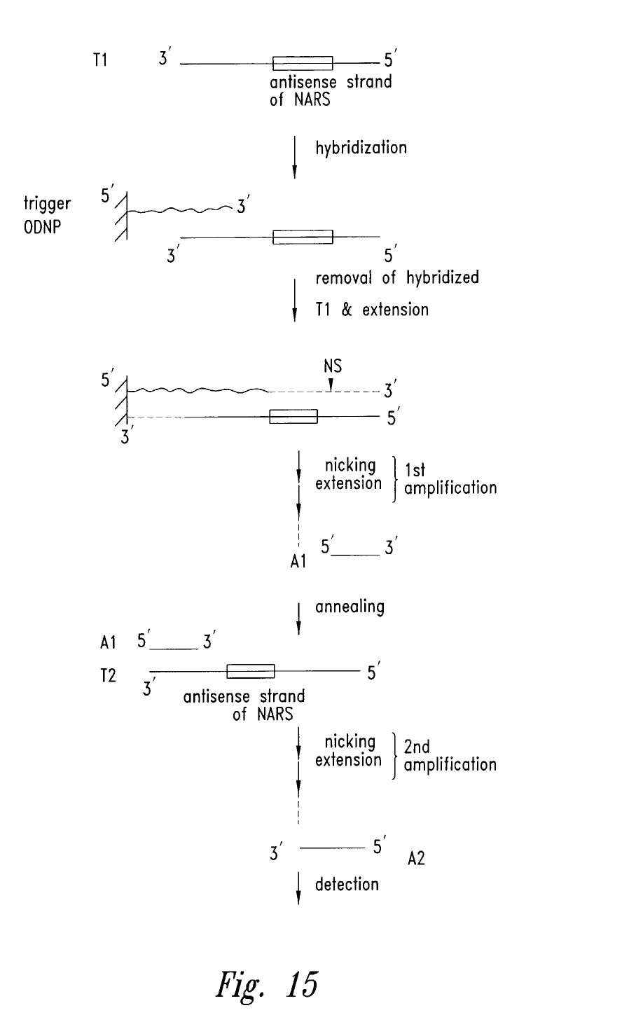

Another example of the above embodiments using a NARS recognizable by a nicking agent that nicks outside the NARS is illustrated in FIG. 15. As shown in this figure, nucleic acids of a biological sample are immobilized via their 5′ termini. The resulting immobilized nucleic acids are then hybridized with a T1 molecule that comprises, from 3′ to 5′, a sequence that is at least substantially complementary to a target nucleic acid suspected to be present in the biological sample and a sequence of the antisense strand of a NARS. If the target nucleic acid is present in the biological sample, the T1 molecule hybridizes to the target nucleic acid to form a N1 molecule. The N1 molecule is separated from unhybridized T1 molecule by washing the solid phase to which the target nucleic acid is attached. In the presence of a DNA polymerase and a nicking agent that recognizes the NARS, N1 is used as a template to amplify a single-stranded nucleic acid molecule A1. However, if the target nucleic acid is absent in the sample, T1 is unable to hybridize to any nucleic acid molecule in the sample and thus is washed off from the solid support. Consequently, no N1 can be formed that attaches to the solid support, and no single-stranded nucleic acid molecule complementary to a portion of N1 can be amplified.

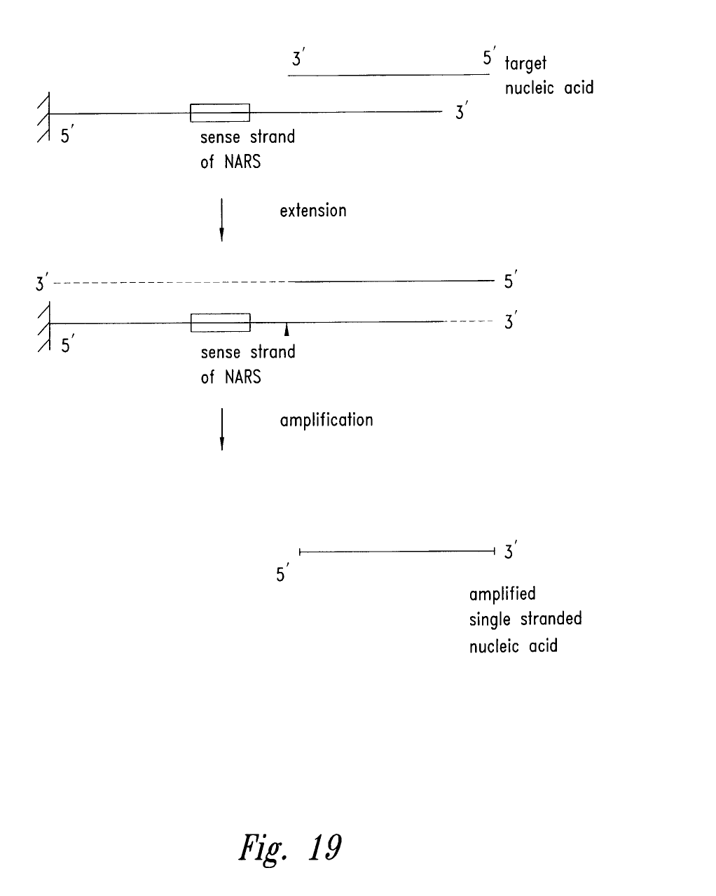

Another example of the above embodiments using a NARS recognizable by a nicking agent that nicks outside the NARS is illustrated in FIG. 19. As shown in this figure, a T1 molecule is immobilized to a solid support via its 5′ terminus. The T1 molecule comprises, from 5′ to 3′, a sequence of the sense strand of the NARS and a sequence that is substantially complementary to the 3′ portion of the target nucleic acid. The T1 molecule is mixed with the nucleic acids from a biological sample. If the target nucleic acid is present in the sample, the T1 molecule is hybridized to the target to form a template molecule. When the solid support to which the T1 molecule is attached is washed, the target remains attached to the solid support via its hybridization with the T1 molecule. In the presence of a DNA polymerase, the target extends from its 3′ terminus using the T1 molecule as a template. The duplex formed between the extension product of the target and that of the T1 molecule comprises a double-stranded NARS. In the presence of a nicking agent that recognizes the NARS as well as the DNA polymerase, a single-stranded nucleic acid molecule is amplified using a portion of the target nucleic acid as a template. However, if the target nucleic acid is absent in the sample, the T1 molecule will not be able to hybridize with the target. Thus, no single-stranded nucleic acid molecule will be amplified using the target as a template.

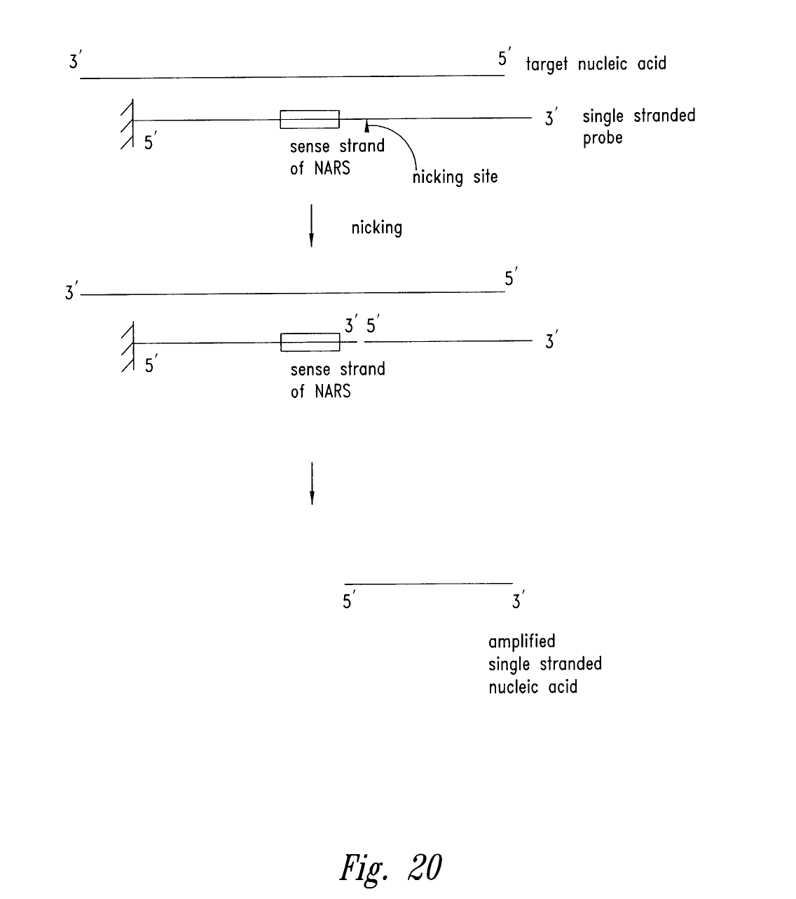

Another example of the above embodiments is illustrated in FIG. 20. In this example, the immobilized T1 molecule is substantially complementary to the target nucleic acid, but not necessarily complementary to the 3′ portion of the target. The T1 also comprises a sequence of the sense strand of a nicking agent recognition sequence. If the target is present in a biological sample, when the T1 molecule is mixed with the nucleic acids in the sample, it may hybridize with the target. When the solid support to which the T1 is attached is washed, the target remains attached to the solid support via its hybridization with the T1. In the presence of a DNA polymerase, and a nicking agent that recognizes the NARS, even when one or more nucleotides in the sequence of the sense strand of the NARS may not form conventional base pairs with nucleotides in the target, in certain circumstances, a single-stranded nucleic acid may be amplified using a portion of the target as a template. The detailed descriptions for the circumstances where a single-stranded nucleic acid is amplified when a template nucleic acid does not comprise a double-stranded NARS are provided in the U.S. Application entitled “Amplification of Nucleic Acid Fragments Using Nicking Agents”. However, if the target nucleic acid is absent in the sample, the probe will not be able to hybridize with the target. Thus, no single-stranded nucleic acid molecule will be amplified using the target as a template.

3. Specificity

The methods of the present invention may be used for detecting the presence or absence of a particular pathogenic organism in a sample, as well as for detecting the presence of several closely related pathogenic organisms. For instance, as to the first and the second types of exemplary methods described above, the portion of a trigger ODNP to which a T1 molecule anneals may be derived from a target nucleic acid or a portion thereof that is specific to a particular pathogenic organism to be detected. Alternatively, such a portion of a trigger ODNP may be derived from a target nucleic acid or a portion thereof that is substantially or completely conserved among several closely related pathogenic organisms, but absent in other more distantly related or unrelated pathogenic organisms.

As used herein, a target nucleic acid or a portion thereof that is “specific” to a particular pathogenic organism refers to a target nucleic acid or a portion thereof having a sequence that is present in the particular organism, not in any other organisms, including those closely related to the particular organism. In addition, as used herein, a region in a target nucleic acid that is “substantially conserved” among several closely related pathogenic organisms refers to a region in the target nucleic acid for which there exists a nucleic acid molecule capable of hybridizing to the corresponding region in each of the several closely related organisms under appropriate conditions, but incapable of hybridizing to a similar region in the target nucleic acid from a more distantly related or unrelated organism under identical conditions. Also, as used herein, a region in a target nucleic acid that is “completely conserved” among several closely related pathogenic organisms refers to a region that has an identical sequence in the target nucleic acid from each of the several closely related pathogenic organisms.

Similarly, as to the above fourth type of exemplary methods, the portion of a target nucleic acid that is amplified with a primer pair may be a region that is specific for a particular pathogenic organism, or a region that is substantially or completely conserved among several closely related pathogenic organisms but absent in other distantly related or unrelated pathogenic organisms. In addition, the amplified portion of a target nucleic acid may be a variable region in the target nucleic acid among several closely related pathogenic organisms. As used herein, a “variable” region in a target nucleic acid refers to a region that has less than 50% sequence identity among the target nucleic acids from closely related organisms, but is surrounded by regions at each side having higher than 80% sequence identity among the target nucleic acids from the same closely related organisms. As used herein, percent sequence identity of two nucleic acids is determined using BLAST programs of Altschul et al. (J. Mol. Biol. 215: 403-10, 1990) with their default parameters. These programs implement the algorithm of Karlin and Altschul (Proc. Natl. Acad. Sci. USA 87:2264-8,1990) modified as in Karlin and Altschul (Proc. Natl. Acad. Sci. USA 90:5873-7, 1993). BLAST programs are available, for example, at the web site http://www.ncbi.nlm.nih.gov.

Likewise, as to the above fifth type of exemplary methods, the overhang of a N1 molecule may be at least substantially complementary to a region in a target nucleic acid specific to a pathogenic organism, or a region in a target nucleic acid that is substantially or completely conserved among several closely related pathogenic organisms. When the overhang is completely complementary to a target nucleic acid or a portion thereof from a particular organism, but also substantially complementary to the target nucleic acid or a portion thereof from one or more closely related organisms, one can vary hybridization stringencies to either detect the presence of the particular organism or to detect the presence of any one of the closely related organisms. For example, when a N1 molecule is hybridized with nucleic acids from a biological sample under highly stringent conditions, nucleic acid amplification following the removal of unhybridized N1 molecules using a portion of the N1 molecule as a template may indicate the presence of the particular organism in the biological sample. On the other hand, when a N1 molecule is hybridized with nucleic acids from a biological sample under moderately or low stringent conditions, nucleic acid amplification (following the removal of unhybridized N1 molecules using a portion of the N1 molecule as a template) may indicate a presence of the particular organism and/or one or more organisms closely related to the particular organism. Adjusting stringencies of hybridization conditions is well known in the art and detailed discussions may be found, for example, Sambrook and Russell, Molecular Cloning: A Laboratory Manual, Cold Spring Harbor Press, 2001.

In the embodiments where an initial nucleic acid molecule (N1) is provided by annealing a trigger ODNP to a T1 molecule, the trigger ODNP or a portion thereof and a portion of the T1 molecule located 3′ to the sequence of one strand of a NARS in T1 may be substantially complementary, rather than completely complementary, to each other. For instance, when a trigger ODNP is derived from a region of a target nucleic acid that is substantially conserved among several closely related pathogenic organisms and the presence of any of the several organisms needs to be detected, a T1 molecule substantially complementary to the trigger ODNP may be used. In such a circumstance, the primer extension reaction needs to be performed under conditions that are not too stringent to prevent the trigger ODNP from annealing to the T1 molecule or prevent the trigger ODNP from being extended using a portion of the T1 molecule as a template. However, such conditions need also be sufficiently stringent to prevent the T1 molecule from non-specifically annealing to a nucleic acid molecule other than the trigger ODNP. Conditions suitable for nucleic acid amplification where a trigger ODNP or a portion thereof is substantially complementary to a portion of a T1 molecule may be worked out by adjusting the reaction temperature and/or reaction buffer composition or concentration. Generally, similar to hybridization reactions, an increase in reaction temperatures increases the stringency of amplification reactions.

4. T2 Molecules

A T2 molecule of the present invention comprises a sequence of the antisense strand of a NARS as well as a sequence, located 3′ to the sequence of the antisense strand of the NARS, that is at least substantially complementary to a single-stranded nucleic acid molecule (A1) amplified using a portion of an initial nucleic acid molecule N1 as a template. Preferably, a T2 molecule comprises a sequence that is completely complementary to an A1 molecule. In certain preferred embodiments, a T2 molecule also comprises a sequence, located 5′ to the sequence of the antisense strand of the NARS, that is either substantially or completely complementary to A1.

Also as discussed above, in the above fourth type of exemplary methods, the portion of a target nucleic acid that is amplified with a primer pair may be a region that is specific for a particular pathogenic organism, or a region that is substantially or completely conserved among several closely related pathogenic organisms but absent in other distantly related or unrelated pathogenic organisms. In addition, the amplified portion of a target nucleic acid may be a variable region in the target nucleic acid among several closely related pathogenic organisms. When the amplified region is substantially conserved, one may use a T2 molecule comprising a sequence, located 3′ to the sequence of the antisense strand of a NARS, that is identical to one strand of the amplified region from a particular organism to detect the presence of the particular organism by performing the amplification reaction under highly stringent conditions (e.g., a relatively high amplification temperature to prevent an A1 molecule derived from an organism other than the particular organism from hybridizing with the T2 molecule). Alternatively, one may use the same T2 molecule to detect the presence of the particular organism as well as the presence of one or more organisms closely related to the particular organism by performing the amplification reaction under moderately or low stringent conditions (e.g., a relatively low amplification temperature to allow an A1 molecule derived from an organism closely related to the particular organism to hybridize with the T2 molecule and to be extended using a portion of the T2 molecule as a template).

Additionally, in the embodiments where the amplified region is a variable region among closely related organisms, a T2 molecule may comprise a sequence that is at least substantially complementary to an A1 molecule amplified using a N1 molecule derived from a particular organism among the above closely related organisms. The amplification of a single-stranded nucleic acid molecule using a portion of the T2 molecule as a template indicates the presence of the particular organism in a biological sample.

5. Detecting and/or Characterizing Amplified Single-Stranded Nucleic Acids

The presence of a target nucleic acid originated from a pathogenic organism may be detected by detecting and/or charactering an amplification product (e.g., A1, A2, etc.). Any method suitable for detecting or characterizing single-stranded nucleic acid molecules may be used. For instance, the amplification reaction may be carried out in the presence of a labeled deoxynucleoside triphosphate so that the label is incorporated into the amplified nucleic acid molecules. Labels suitable for incorporating into a nucleic acid fragment, and methods for the subsequent detection of the fragment are known in the art, and exemplary labels include, but are not limited to, a radiolabel such as ³²P, ³³P, ¹²⁵I or ³⁵S, an enzyme capable of producing a colored reaction product such as alkaline phosphatase, fluorescent labels such as fluorescein isothiocyanate (FITC), biotin, avidin, digoxigenin, antigens, haptens, or fluorochromes.

Alternatively, amplified nucleic acid molecules may be detected by the use of a labeled detector oligonucleotide that is substantially, preferably completely, complementary to the amplified nucleic acid molecules. Similar to a labeled deoxynucleoside triphosphate, the detector oligonucleotide may also be labeled with a radioactive, chemiluminescent, or fluorescent tag (including those suitable for detection using fluorescence polarization or fluorescence resonance energy transfer), or the like. See, Spargo et al., Mol. Cell. Probes 7: 395-404, 1993; Hellyer et al., J. Infectious Diseases 173: 934-41,1996; Walker et al., Nucl. Acids Res. 24: 348-53,1996; Walker et al., Clin. Chem. 42: 9-13,1996; Spears et al., Anal. Biochem. 247:130-7,1997; Mehrpouyan et al., Mol. Cell. Probes 11: 337-47,1997; and Nadeau et al., Anal. Biochem. 276:177-87,1999.

In certain embodiments, amplified nucleic acid molecules may be further characterized. The characterization may confirm the identities of these nucleic acid molecules and thus confirm the presence of a target nucleic acid from a pathogenic organism in a biological sample. Such a characterization may be performed via any known method suitable for characterizing single-stranded nucleic acid fragments. Exemplary techniques include, without limitation, chromatography such as liquid chromatography, mass spectrometry and electrophoresis. Detailed description of various exemplary methods may be found in U.S. Prov. Appl. Nos. 60/305,637 and 60/345,445, incorporated herein in their entireties.

Besides detecting and/or characterizing an amplification product to detect the presence of a target nucleic acid in a biological sample, the presence of the target nucleic acid may be detected by detecting completely or partially double-stranded nucleic acid molecules produced in the amplification reactions (e.g., H1, H2 or nicking product thereof). In a preferred embodiment, the detection of the double-stranded nucleic acid molecule may be performed by adding to the amplification mixture a dye that specifically binds to double-stranded nucleic acid molecules and becomes fluorescent upon binding to double-stranded nucleic acid molecules (i.e., fluorescent intercalating agent). The addition of a fluorescent intercalating agent enables real time monitoring of nucleic acid amplification. Alternatively, to maximize the production of double-stranded nucleic acid molecules (e.g., H1 and H2), the NE, but not the DNA polymerase, in the nicking-extension reaction mixture may be inactivated (e.g., by heat treatment). The inactivation of the NE allows all the nicked nucleic acid molecules in the reaction mixture to be extended to produce double-stranded nucleic acid molecules.

Various fluorescent intercalating agents are known in the art and may be used in the present invention. Exemplary agents include, without limitation, those disclosed in U.S. Pat. Nos. 4,119,521; 5,599,932, 5,658,735; 5,734,058; 5,763,162; 5,808,077; 6,015,902; 6,255,048 and 6,280,933, those discussed in Glazer and Rye, Nature 359: 859-61,1992, PicoGreen dye, and SYBR® dyes such as SYBR® Gold, SYBR® Green I and SYBR® Green II (Molecular Probes, Eugene Wash.). Fluorescence produced by fluorescent intercalating agents may be detected by various detectors, including PMTs, CCD cameras, fluorescent-based microscopes, fluorescent-based scanners, fluorescent-based microplate readers, fluorescent-based capillary readers.

6. Compositions and Kits Useful in Diagnosis

Compositions and kits useful in pathogen diagnosis may be same as those described above for exponential amplification of nucleic acids. In certain embodiments, these compositions and kits may further comprise an additional component to facilitate the detection of amplification products. For instance, the additional component may be a labeled deoxynucleoside triphosphate to be incorporated into amplification products. Alternatively, it may be a labeled detector oligonucleotide. In certain preferred embodiments, the additional component may be a fluorescent intercalating agent.

7. Diagnostic Uses of the Present Invention

The present invention is useful in quickly detecting the presence of any target nucleic acid of interest. In certain embodiments, the target nucleic acid is derived or originated from a pathogenic organism (e.g., an organism that causes infectious diseases). Such pathogenic organisms include those that impose bio-threat, such as Anthrax and smallpox. In addition, as described above, the present methods may be used for the detecting the presence of a particular pathogenic organism as well as for detecting the presence of several closely related pathogenic organisms. The present invention may also be used to detect organisms that are resistant to certain antibiotics. For example, the present methods, compositions or kits may be used to detect certain pathogenic organisms in a subject that has been treated with an antibiotic or certain combinations of antibiotics. Furthermore, the use of fluorescent intercalating agents for detecting nucleic acid amplification in some embodiments offers real time detection of a target nucleic acid in a biological sample.

D. Use of Nucleic Acid Amplification Methods and Compositions in Genetic Variation Detection

The methods and compositions for exponential nucleic acid amplification may also be used for detecting genetic variations at defined locations in target nucleic acids. A target nucleic acid or its portion that comprises a genetic variation is first incorporated into an initial nucleic acid molecule (N1) to be used as a template in a first amplification reaction. The initial nucleic acid molecule also comprises at least one strand of a first nicking agent recognition sequence and thus allows for the first amplification reaction in the presence of a DNA polymerase and a nicking agent that recognizes the first nicking agent recognition sequence. The product (A1) from the first amplification reaction comprises the nucleotide(s) at the defined location in the target nucleic acid or the complementary nucleotide(s) of the above nucleotide(s).

In certain embodiments, it is desirable to determine whether a target nucleic acid contains a specific genetic variation. In such embodiments, a single-stranded template nucleic acid T2 is used to anneal to the A1 molecule amplified as described above. The T2 molecule comprises, from 3′ to 5′: (a) a first sequence that is at least substantially complementary to the A1 and comprises the specific genetic variation or the complementary thereof, (b) a sequence of the antisense strand of a nicking agent recognition sequence that is recognizable by a second nicking agent, and (c) a second sequence. An amplification reaction is then performed under conditions that amplify a single-stranded nucleic acid molecule (A2) using at least a portion of the second sequence of the T2 molecule only if the A1 comprises the specific genetic variation or the complement thereof. The reaction mixture is then analyzed to determine whether any A2 molecules have been amplified. The presence of the A2 molecules in the mixture indicates that the target nucleic acid contains the specific genetic variation at the defined location, whereas the absence of the A2 molecules indicates that the target nucleic acid does not contain the specific genetic variation.

In certain other embodiments, it may be desirable to identify a genetic variation at a defined location in a target nucleic acid. In such embodiments, multiple single-stranded template nucleic acids (T2 molecules) are used to contact the A1 molecule amplified as described above. The T2 molecules each comprise, from 3′ to 5′: (a) a first sequence that is at least substantially complementary to the A1 molecule and comprises one of the potential genetic variations at the defined position of the target nucleic acid or the complement of the potential genetic variation, (b) a sequence of the antisense strand of a nicking agent recognition sequence that is recognizable by a second nicking agent, and (c) a second sequence that uniquely correlates to the potential genetic variation. A sequence that “uniquely correlates to” a potential genetic variation refers to a sequence that is present in a T2 molecule that comprises a specific potential genetic variation of a target nucleic acid or the complement of the specific potential genetic variation, but is absent in a T2 molecule that comprises another potential genetic variation of the target nucleic acid or the complement of the other potential genetic variation. The multiple T2 molecules, in combination, comprise some or all of the potential genetic variations at the defined location of the target nucleic acid or the complements of some or all of the potential genetic variations.

After the A1 is mixed with the multiple T2 molecules, an amplification reaction is then performed under conditions that selectively amplify a single-stranded nucleic acid molecule (A2) using a portion of the second sequence of a T2 molecule that comprises the genetic variation of the target nucleic acid or the complement of the genetic variation as a template. The amplified A2 molecule is then characterized to determine which T2 molecule was used as the template for the amplification of the A2 molecule. The identification of the T2 molecule that functioned as the template indicates that the target nucleic acid comprises the genetic variation that is present in the first sequence of the T2 molecule if the A1 molecule comprises the complement of the genetic variation of the target, or the target nucleic acid comprises the genetic variation whose complement is present in the first sequence of the T2 molecule if the A1 molecule comprises the genetic variation of the target nucleic acid.

1. Target Nucleic Acids

The target nucleic acid of the present invention related to identifying genetic variations is any nucleic acid molecule that may contain a genetic variation using a wild type nucleic acid sequence as a reference. It may or may not be immobilized to a solid support. It can be either single-stranded or double-stranded. A single-stranded target nucleic acid may be one strand of a denatured double-stranded DNA. Alternatively, it may be a single-stranded nucleic acid not derived from any double-stranded DNA. In one aspect, the target nucleic acid is DNA, including genomic DNA, ribosomal DNA and cDNA. In another aspect, the target is RNA, including mRNA, rRNA and tRNA.

In one aspect, the target nucleic acid either is or is derived from naturally occurring nucleic acid. A naturally occurring target nucleic acid is obtained from a biological sample. Preferred biological samples include one or more mammalian tissues, preferably human tissues, (for example blood, plasma/serum, hair, skin, lymph node, spleen, liver, etc.) and/or cells or cell lines. The biological samples may comprise one or more human tissues and/or cells. Mammalian and/or human tissues and/or cells may further comprise one or more tumor tissues and/or cells.

Methodology for isolating populations of nucleic acids from biological samples is well known and readily available to those skilled in the art of the present invention. Exemplary techniques are described, for example, in following laboratory research manuals: Sambrook et al., “Molecular Cloning” (Cold Spring Harbor Press, 3rd Edition, 2001) and Ausubel et al., “Short Protocols in Molecular Biology” (1999) (incorporated herein by reference in their entireties). Nucleic acid isolation kits are also commercially available from numerous companies, and may be used to simplify and accelerate the isolation process.

The target nucleic acid contains one or more nucleotides of unknown identity (i.e., genetic variations). The present invention provides compositions and methods whereby the identity of the unknown nucleotide(s) becomes known and thereby the genetic variation becomes identified. The base(s) of unknown identity is present at the “nucleotide locus” (or the “defined position” or the “defined location”), which refers to a specific nucleotide or region encompassing one, two, three, four, five, six, seven, or more nucleotides having a precise location on a target nucleic acid.

The term “polymorphism” refer to the occurrence of two or more genetically determined alternative sequences or alleles in a small region (i.e., one to several (e.g., 2, 3, 4, 5, 6, 7, or 8) nucleotides in length) in a population. The two or more genetically determined alternative sequences or alleles each may be referred to as a “genetic variation.” The genetic variation may be the allelic form occurring most frequently in a selected population also referred to as “the wild type form” or one of the other allelic forms. Diploid organisms may be homozygous or heterozygous for allelic forms.

Genetic variations may or may not have effects on gene expression, including expression levels and expression products (i.e., encoded peptides). Genetic variations that affect gene expression are also referred to as “mutations,” including point mutations, frameshift mutations, regulatory mutations, nonsense mutations, and missense mutation. A “point mutation” refers to a mutation in which a wild-type base (i.e., A, C, G, or T) is replaced with one of the other standard bases at a defined nucleotide locus within a nucleic acid sample. It can be caused by a base substitution or a base deletion. A “frameshift mutation” is caused by small deletions or insertions that, in turn, cause the reading frame(s) of a gene to be shifted and, thus, a novel peptide to be formed. A “regulatory mutation” refers to a mutation in a non-coding region, e.g., an intron, a region located 5′ or 3′ to the coding region, that affects correct gene expression (e.g., amount of product, localization of protein, timing of expression). A “nonsense mutation” is a single nucleotide change resulting in a triplet codon (where mutation occurs) being read as a “STOP” codon causing premature termination of peptide elongation, i.e., a truncated peptide. A “missense mutation” is a mutation that results in one amino acid being exchanged for a different amino acid. Such a mutation may cause a change in the folding (3-dimensional structure) of the peptide and/or its proper association with other peptides in a multimeric protein.

In one aspect of the invention, the genetic variation is a “single-nucleotide polymorphism” (SNP), which refers to any single nucleotide sequence variation, preferably one that is common in a population of organisms and is inherited in a Mendelian fashion. Typically, the SNP is either of two possible bases and there is no possibility of finding a third or fourth nucleotide identity at an SNP site.

The genetic variation may be associated with or cause diseases or disorders. The term “associated with,” as used herein, refers to the presence of a positive correlation between the occurrence of the genetic variation and the presence of a disease or a disorder in the host. Such diseases or disorders may be human genetic diseases or disorders and include, but are not limited to, cystic fibrosis, bladder carcinoma, colorectal tumors, sickle-cell anemia, thalassemias, alantitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease.

Target nucleic acids may be amplified before being incorporated into initial nucleic acids as described below. Any of the known methods for amplifying nucleic acids may be used. Exemplary methods include, but are not limited to, the use of Qbeta Replicase, Strand Displacement Amplification (Walker et al., Nucleic Acid Research 20:1691-6,1995), transcription-mediated amplification (Kwoh et al., PCT Int'l. Pat. Appl. Pub. No.WO88/10315), RACE (Frohman, Methods Enzymol. 218:340-56, 1993), one-sided PCR (Ohara et al., Proc. Natl. Acad. Sc. 86: 5673-7,1989), and gap-LCR (Abravaya et al., Nucleic Acids Res. 23: 675-82, 1995). The cited articles and the PCT international patent application are incorporated herein by reference in their entireties.

2. Initial Nucleic Acid Molecules (N1)

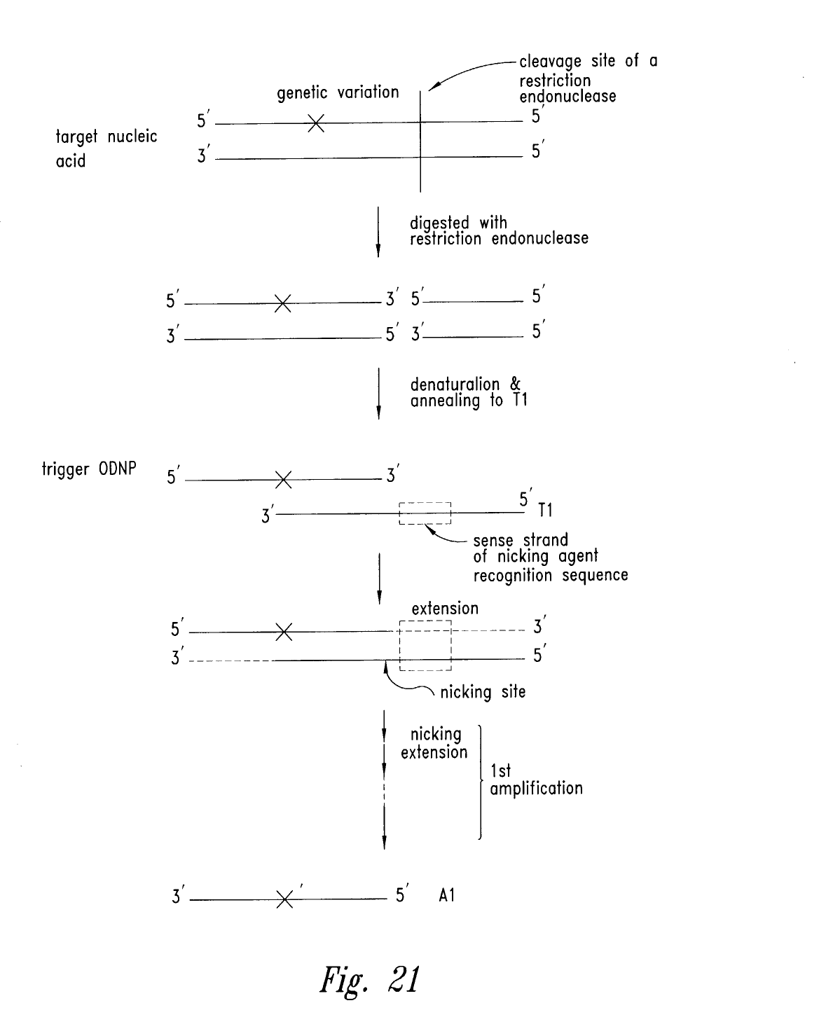

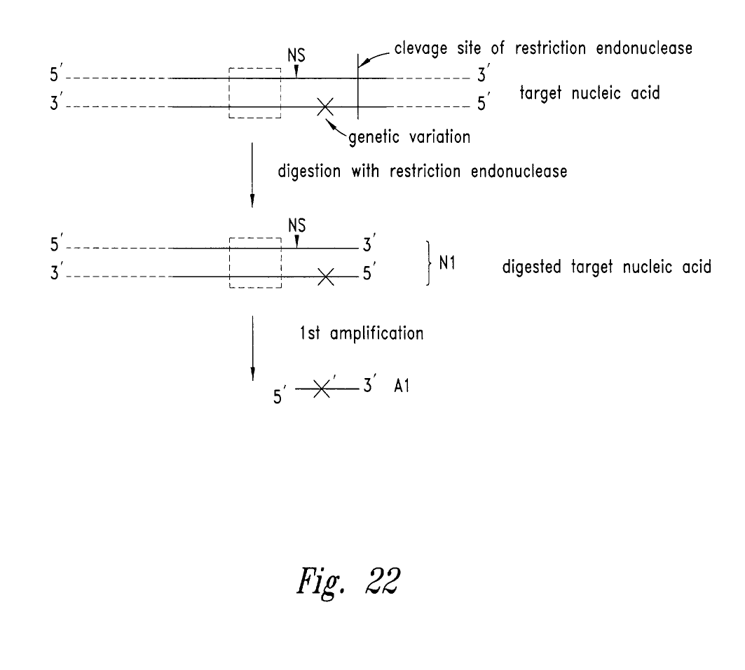

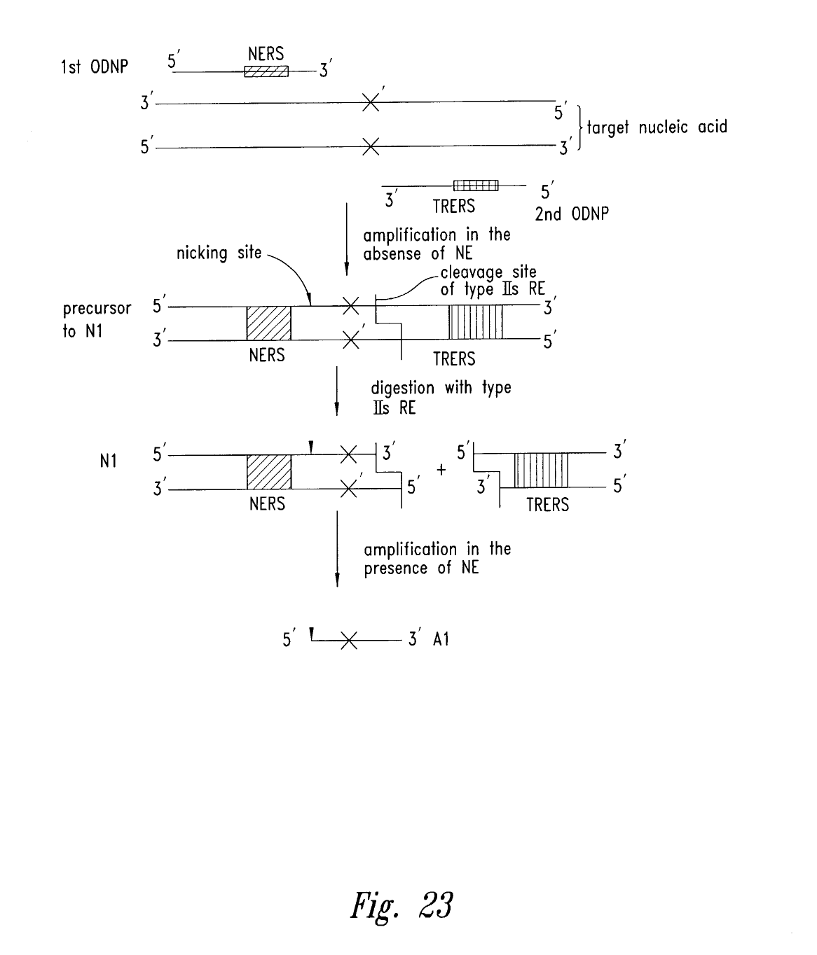

Initial nucleic acid molecules useful for genetic variation detection may be provided by various approaches. For instance, N1 may be obtained by annealing of a trigger oligonucleotide primer to a T1 molecule where the trigger primer is derived from a target nucleic acid and encompasses a genetic variation in the target nucleic acid (e.g., FIG. 21). Alternatively, N1 may be directly derived from a double-stranded target nucleic acid (e.g., by digestion of the target nucleic acid with a restriction endonuclease as shown in FIG. 22). N1 may also be prepared by the use of appropriate oligonucleotide primer pairs (e.g., FIGS. 23-25). Several exemplary means for providing initial nucleic acid molecules are described below.

a. First Type of Exemplary Methods for Providing N1 Molecules

As noted above, N1 may be provided by annealing a trigger oligonucleotide primer to a T1 molecule. The trigger primer needs to encompass genetic variation of a target nucleic acid. An example of this type of methods for providing N1 molecules is illustrated in FIG. 21. As shown in this figure, a double-stranded target nucleic acid (e.g., a genomic DNA) is first cleaved by a restriction endonuclease whose recognition sequence is close to the defined location where a genetic variation is present. The digestion products may be denatured and the strand of the digestion product that comprises the potential genetic variation may then be used as a trigger oligonucleotide primer to anneal to a template nucleic acid (T1). T1 comprises a sequence of the sense strand of a nicking agent recognition sequence so that in the presence of a DNA polymerase and a nicking agent that recognizes the recognition sequence, a single-stranded nucleic acid fragment (A1) is amplified that comprises the complementary nucleotide(s) of the genetic variation of the target nucleic acid.

b. Second Type of Exemplary Methods for Providing N1 Molecules

In certain embodiments of the present invention, N1 is directly derived from a target nucleic acid that comprises a potential genetic variation, a nicking agent recognition sequence, and a restriction endonuclease recognition sequence. An embodiment with a recognition sequence recognizable by a nicking endonuclease that nicks outside its recognition sequence (e.g., N.BstNB I) as an exemplary nicking agent recognition sequence is illustrated in FIG. 22. As shown in this figure, a target nucleic acid may be digested by a restriction endonuclease that recognizes a sequence in the target nucleic acid. The digestion product that contains the nicking endonuclease recognition sequence may function as an initial nucleic acid molecule (N1) to amplify a single-stranded nucleic acid fragment (A1). The genetic variation (“X”) needs to be between the position corresponding to the nicking site produced by the nicking agent and the restriction cleavage site of the restriction endonuclease. Such a location allows the amplified fragment (A1) to contain the complement (“X”′) of the genetic variation.

C. Third Type of Exemplary Methods for Providing N1 Molecules

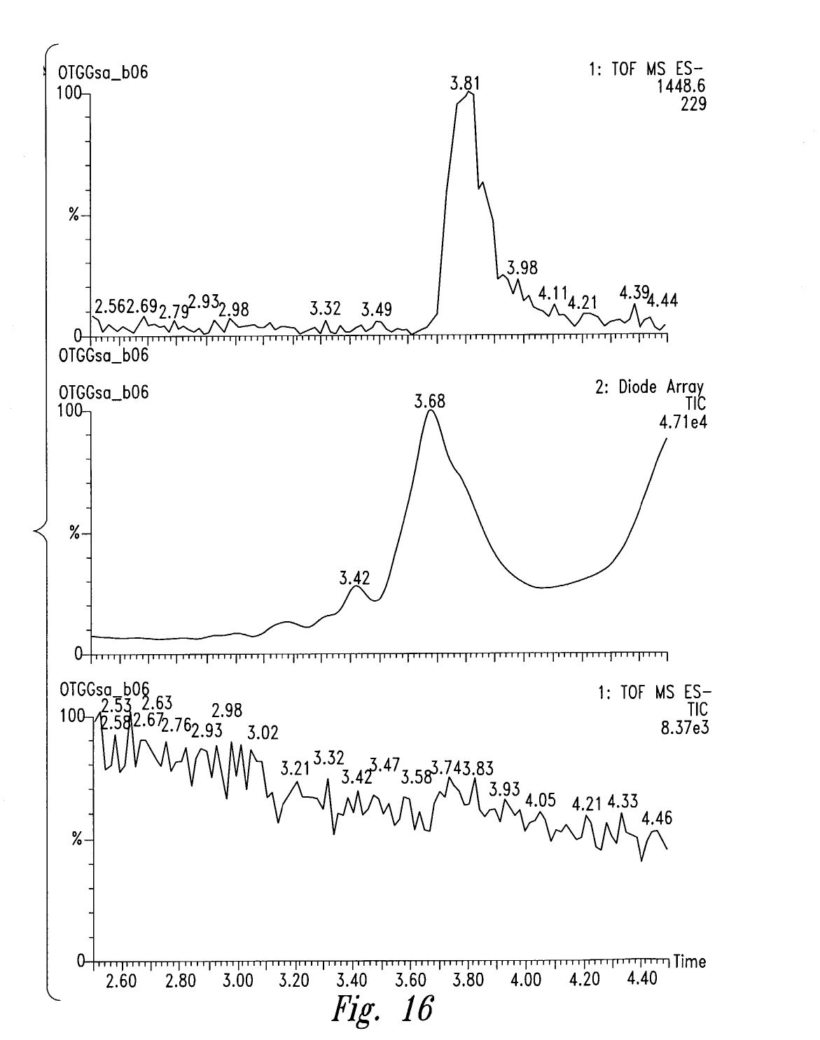

In certain embodiments of the present invention, an initial nucleic acid molecule N1 is a completely or partially double-stranded nucleic acid molecule produced using various ODNP pairs. The methods for using ODNP pairs to prepare N1 molecules are briefly described below in connection with FIGS. 16-18. More detailed description may be found in U.S. Prov. Appl. Nos. 60/305,637 and 60/345,445.

In certain embodiments, a precursor to N1 contains a double-stranded nicking agent recognition sequence and a restriction endonuclease recognition sequence. The nicking agent recognition sequence and the restriction endonuclease recognition sequence are incorporated into the precursor using a primer pair. An embodiment with a recognition sequence recognizable by a nicking agent that nicks outside its recognition sequence (e.g., N.BstNB I) as an exemplary nicking agent recognition sequence, and a type IIs restriction endonuclease recognition sequence (TRERS) as an exemplary restriction endonuclease recognition sequence is illustrated in FIG. 23. As shown in this figure, a first primer comprises the sequence of one strand of a nicking agent recognition sequence, while a second ODNP comprises the sequence of one strand of a type IIs restriction endonuclease recognition sequence. When these two ODNPs are used as primers to amplify a portion of a target nucleic acid, the resulting amplification product (i.e., a precursor to N1), contains both a double-stranded NERS and a double-stranded TRERS. In addition, the first primer is designed to anneal to a portion of one strand of the target nucleic acid located 3′ to the complement of a genetic variation, whereas the second primer is designed to anneal to a portion of the other strand of the target nucleic acid located 3′ to the genetic variation. Such designs allow the precursor to N1 to encompass the genetic variation and its complement. In the presence of a type IIs restriction endonuclease that recognizes the TRERS, the amplification product is digested to produce a partially double-stranded nucleic acid molecule N1 that comprises a double-stranded NERS.

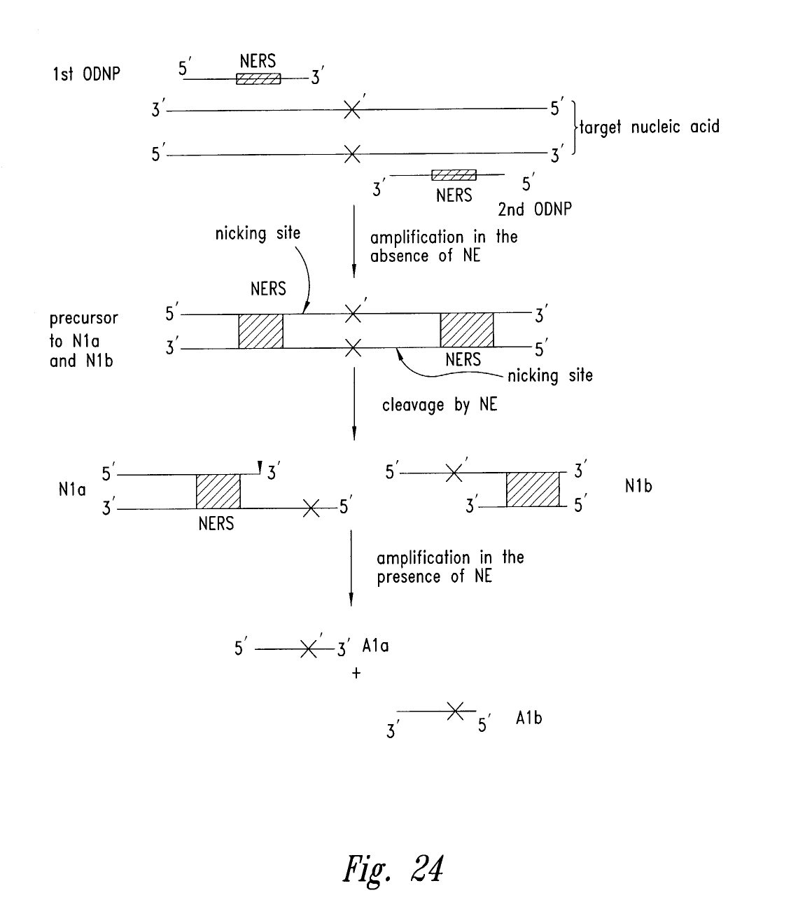

In other embodiments, a precursor to N1 contains two double-stranded nicking agent recognition sequences. The two nicking agent recognition sequences are incorporated into the precursor to N1 using two oligonucleotide primers. An embodiment with a recognition sequence recognizable by a nicking endonuclease that nicks outside its recognition sequence as an exemplary nicking agent recognition sequence is illustrated in FIG. 24. As shown in this figure, both primers comprise a sequence of a sense strand of a nicking endonuclease recognition sequence. In addition, the first primer is designed to anneal to a portion of one strand of the target nucleic acid located 3′ to the complement of a genetic variation, whereas the second primer is designed to anneal to a portion of the other strand of the target nucleic acid located 3′ to the genetic variation. When these two primers are used as primers to amplify a portion of a target nucleic acid, the resulting amplification product (i.e., a precursor to N1a and N1b described below) contains the genetic variation and its complement, as well as two nicking endonuclease recognition sequences. These two recognition sequences may or may not be identical to each other, but preferably, they are identical. In the presence of a nicking endonuclease or nicking endonucleases that recognize the recognition sequences, the amplification product is nicked twice (once on each strand) to produce two partially double-stranded nucleic acid molecules (N1a and N1b) that each comprises one of the double-stranded nicking endonuclease recognition sequences.

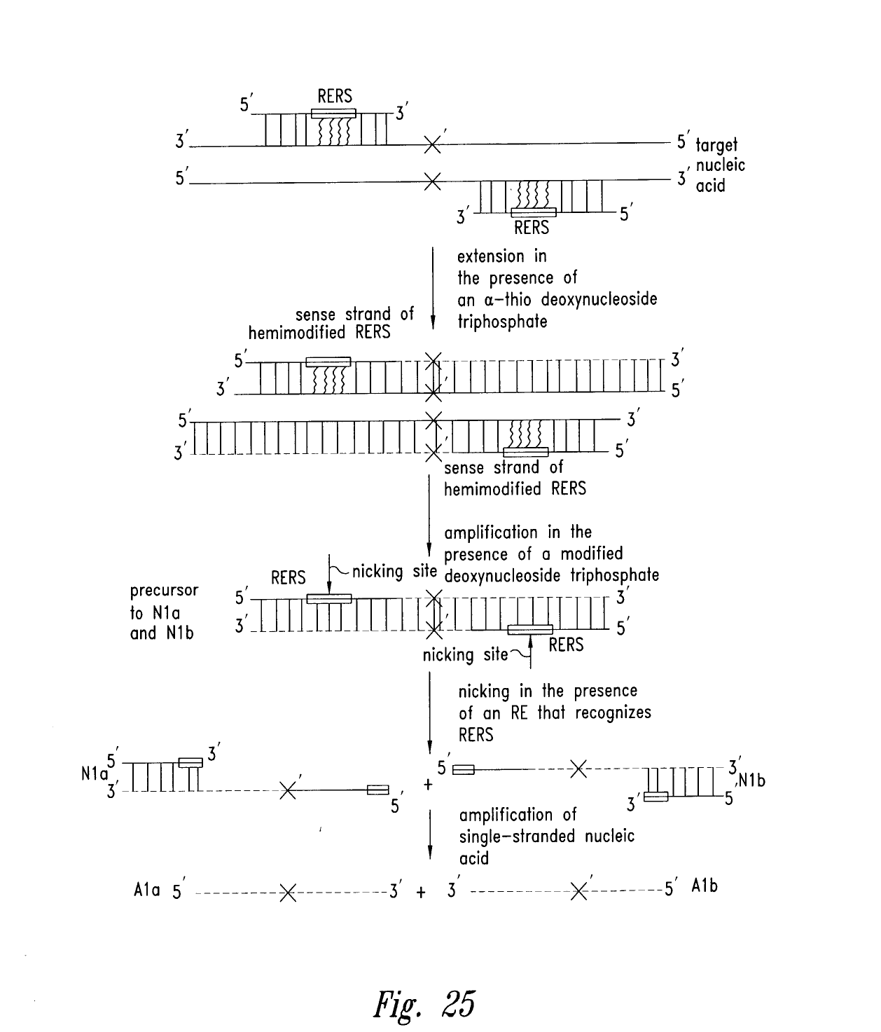

Another embodiment with a hemimodified restriction endonuclease recognition sequence as an exemplary nicking agent recognition sequence is illustrated in FIG. 25. As shown in this figure, both the first and the second primers comprise a sequence of one strand of a restriction endonuclease recognition sequence. In addition, the first primer is designed to anneal to a portion of one strand of the target nucleic acid located 3′ to the complement of a genetic variation, whereas the second primer is designed to anneal to a portion of the other strand of the target nucleic acid located 3′ to the genetic variation. When these two primers are used as primers to amplify a portion of a target nucleic acid in the presence of a modified deoxynucleoside triphosphate, the resulting amplification product (i.e., a precursor to N1a and N1b described below) contains the genetic variation and its complement, as well as two hemimodified restriction endonuclease recognition sequences. These two hemimodified recognition sequences may or may not be identical to each other. In the presence of a restriction endonuclease or restriction endonucleases that recognize the hemimodified recognition sequences, the above amplification product is nicked to produce two partially double-stranded nucleic acid molecules (N1a and N1b) that each comprises a sequence of at least one strand of one of the hemimodified restriction endonuclease recognition sequences.

The above first ODNP, the second ODNP or both may be immobilized to a solid support in certain embodiments. In other embodiments, the nucleic acid molecules of a sample, including the target nucleic acid are immobilized.

3. A1 Molecules

As described above, an A1 molecule is amplified using a portion of N1 as a template. This portion of N1 comprises the genetic variation or its complement of the target nucleic acid so that A1 comprises the complement of the genetic variation or the genetic variation itself. A1 may be relatively short and has at most 25, 20, 17, 15, 10, or 8 nucleotides. Such short length may be accomplished by appropriately designing oligonucleotide primers used in making N1 molecules. For instance, for the third type of providing N1 molecules (FIGS. 16-18), the ODNP pair may be designed to be close to each other when they anneal to the target nucleic acid. Similar to the diagnostic application of the present invention described above, the short length of an A1 molecule increases amplification efficiencies and rates, allows the use of a DNA polymerase that does not have a stand displacement activity, and facilitates the detection of A1 molecules and/or a product (A2) of a subsequent amplification reaction in which the A1 is used as an initial amplification primer via certain technologies such as mass spectrometric analysis. Further, the short length of an A1 allows for easier identification of reaction conditions under which an A1 only hybridizes to a T2 molecule that comprises a nucleotide (or nucleotides) that is complementary to the nucleotide (or nucleotides) in the A1 molecule that is derived from the genetic variation of the target nucleic acid.

4. T2 Molecules

As described above, a T2 molecule of the present invention comprises a sequence of the antisense strand of a nicking agent recognition sequence as well as a sequence, located 3′ to the sequence of the sense strand of the recognition sequence, that is at least substantially complementary to a single-stranded nucleic acid molecule (A1) amplified using a portion of an initial nucleic acid molecule N1 as a template. In certain embodiments where it is desirable to determine whether a target nucleic acid contains a specific genetic variation, the T2 molecule may comprise, from 3′ to 5′: (a) a first sequence that is at least substantially complementary to the A1 and comprises the specific genetic variation or the complementary thereof, (b) a sequence of the antisense strand of a nicking agent recognition sequence that is recognizable by a second nicking agent, and (c) a second sequence.

In other embodiments where it is desirable to identify a genetic variation at a defined location in a target nucleic acid, multiple single-stranded template nucleic acids (T2 molecules) are used. The T2 molecules each comprise, from 3′ to 5′: (a) a first sequence that is at least substantially complementary to the A1 molecule and comprises one of the potential genetic variations at the defined position of the target nucleic acid or the complement of the potential genetic variation, (b) a sequence of the antisense strand of a nicking agent recognition sequence that is recognizable by a second nicking agent, and (c) a second sequence that uniquely correlates to the potential genetic variation. The multiple T2 molecules, in combination, comprise some or all of the potential genetic variation at the defined location of the target nucleic acid or the complements of all the potential genetic variations.

In certain embodiments, for one or more, preferably, all of the multiple T2 molecules, the second sequences of a T2 molecule may be at least substantially identical to the first sequences of the same T2 molecule so that an amplification product (A2) using the second sequence as a template is identical to the A1 molecule that anneals to the first sequence. Thus, the characterization of the A2 molecule may directly indicate the identity of the A1 molecule, and accordingly, the identity of the genetic variation in the target nucleic acid.

The T2 molecule may be immobilized to a solid support, preferably via its 5′ terminus, in certain embodiments. In addition, multiple immobilized T2 molecules may form an array. In other embodiments, the T2 molecule may not be immobilized.

5. Reaction Conditions

As noted above, as to the embodiments for determining the presence or absence of a specific genetic variation in a target nucleic acid, an amplification reaction is performed under conditions that amplify an A2 using at least a portion of the second sequence of the T2 molecule only if the A1 comprises the specific genetic variation or the complement thereof. Methods for identifying conditions that allow for a molecule (e.g., a A1 molecule) to selectively anneal to one of multiple molecules (e.g., T2 molecules) that have identical sequences except at a defined location and to initiate a primer extension are known in the art. For instance, such conditions may be worked out by varying the reaction temperature, the length of a A1 molecule, the composition of the reaction mixture, or the like. Generally, the higher the reaction temperature, the higher stringency of the hybridization between an A1 and the first sequence of a T2 molecule. In addition, the shorter an A1 molecule to the extent that still allows for the hybridization between the A1 molecule and a T2 molecule that has the complement of the nucleotide(s) in the A1 molecule that is derived from the nucleotides at the defined position of a target nucleic acid, the easier for identifying conditions under which the A1 molecule hybridizes with the above T1 molecule, but not with another T1 molecule that is identical to the above T1 molecule except that it does not have the complement of the nucleotide(s) in the A1 molecule that is derived from the nucleotides at the defined position of the target nucleic acid. Further, suitable reaction conditions may be optimized or verified using one or more target nucleic acids with a known genetic variation as control(s).

At to the embodiments for identifying a genetic variation at a defined location in a target nucleic acid, an amplification reaction is performed in the presence of multiple T2 molecules. These T2 molecules, in combination, comprise some or all of the potential genetic variations at the defined location of the target nucleic acid or the complements of all the potential genetic variations. The amplification reaction is carried out under conditions that selectively amplify a single-stranded nucleic acid molecule (A2) using a portion of the second sequence of a T2 molecule that comprises the genetic variation of the target nucleic acid or the complement of the genetic variation as a template. In other words, only the above A2 molecule is amplified, and no A2 molecule is amplified using a portion of another T2 molecule that is identical to the above T2 molecule except that it does not comprise the genetic variation or its complement in the target. Such reaction conditions may be identified similar to those for determining the presence or absence of a specific genetic variation in a target nucleic acid: They also allow selective hybridization between an A1 molecule and a T1 molecule that comprises the complement of the nucleotide(s) in the A1 molecule that is derived from the genetic variation of the target nucleic acid.

6. Characterizing Amplified Single-Stranded Nucleic Acids

A potential genetic variation in a target nucleic acid may be detected or identified by characterizing an amplification product (i.e., A1 or A2). Any method suitable for characterizing single-stranded nucleic acid molecules may be used. Exemplary techniques include, without limitation, chromatography such as liquid chromatography, mass spectrometry and electrophoresis. Detailed description of various exemplary methods may be found in U.S. Prov. Appl. Nos. 60/305,637 and 60/345,445.

Many of the methodologies for characterizing amplified single-stranded nucleic acid fragments may also be used to measure the amount of a particular amplified single-stranded nucleic acid fragment in the amplification reaction mixture. For instance, in the embodiments where an amplified single stranded nucleic acid molecule is first separated from the other molecules in the amplification reaction mixture by liquid chromatography and then subject to mass spectrometry analysis, the amount of the amplified single-stranded nucleic acid molecule may be quantified either by liquid chromatography of the fraction that contains the nucleic acid molecule, or by ion current measurement of the mass spectrometry peak corresponding to the nucleic acid molecule.

Such methodologies may be used to determine the allelic frequency of a target nucleic acid in a population of nucleic acids where the allelic variant(s) of the target nucleic acid may also be present. “Allelic variant” refers to a nucleic acid molecule that has an identical sequence to the target nucleic acid except at a defined location of the target nucleic acid. “Allelic frequency of a target nucleic acid in a population of nucleic acids” refers to the percentage of the total amount of the target nucleic acid and its allelic variant(s) in the nucleic acid population that is the target nucleic acid. Because the primer pairs used in preparing precursors to N1 are designed to anneal to portions of a target nucleic acid at each side of a potential genetic variation at a defined location in the target, the amplification using the primer pairs as primers and a nucleic acid population containing the target nucleic acid as templates produces the nucleic acid fragment that contains the genetic variation at the defined location of the target nucleic acid, as well as the nucleic acid fragment(s) that contains the genetic variations at the same location of the allelic variant(s) of the target nucleic acid if the variant(s) is present in the nucleic acid population. Because the sequences of the target nucleic acid and its allelic variant(s) differ only at the defined location, the precursors to N1 using the target nucleic acid and the allelic variant(s) as respective templates are amplified at an identical, or a similar, efficiency. Likewise, the single-stranded nucleic acid molecules (A1) that contain the genetic variation or its complement of the target nucleic acid are amplified at the efficiency identical or similar to that of the single-stranded nucleic acid molecules that contain the genetic variation or its complement of the allelic variants. In addition, if a T2 molecule is used that anneals to the A1 molecules amplified using the target and its allelic variants as respective templates at a same efficiency, the ratio of the A2 molecules amplified with the target as an initial template to the A2 molecules amplified using the variant(s) as an initial template reflects the ratio of the target to its variant(s) in the nucleic acid population. Thus, the measurement of the relative amount of A1 (or A2) molecules in the reaction mixture indicates the relative amount of the target nucleic acid in the nucleic acid population.

7. Compositions and Kits Useful in Genetic Variation Detection

Compositions and kits useful in genetic variation detection may be the same as those described above for exponential nucleic acid amplification. In certain embodiments, these kits may further comprise one or more additional components useful in characterizing amplification products. For instance, the additional component may be (1) a chromatography column; (2) a buffer for performing chromatographic characterization or separation of nucleic acids; (3) microtiter plates or microwell plates; (4) oligonucleotide standards (e.g., 6 mer, 7 mer, 8 mer, 10 mer, 12 mer, 14 mer and 16 mer) for liquid chromatography and/or mass spectrometry; and (5) an instruction booklet for using the kits.

8. Applications of the Present Genetic Variation Detection Methods

As described in detail above, the present invention provides methods for detecting and/or identifying genetic variations in target nucleic acids. Methods according to the present invention may find utility in a wide variety of applications where it is desirable or necessary to identify or measure genetic variations. Such applications include, but are not limited to, genetic analysis for hereditarily transferred diseases, tumor diagnosis, disease predisposition, forensics, paternity determination, enhancements in crop cultivation or animal breeding, expression profiling of cell function and/or disease marker genes, and identification and/or characterization of infectious organisms that cause infectious diseases in plants or animal and/or that are related to food safety.

For instance, the present invention may be useful in genetic analysis for forensic purposes. The identification of individuals at the level of DNA sequence variations is advantageous over conventional criteria such as fingerprints, blood type or physical characteristics. In contrast to most phenotypic markers, DNA analysis readily permits the deduction of relatedness between individuals such as is required in paternity testing. Genetic analysis has proven highly useful in bone marrow transplantation, where it is necessary to distinguish between closely related donor and recipient cells. The present invention is useful in characterizing polymorphism of sample DNAs, therefore useful in forensic DNA analysis. For example, the analysis of 22 separate gene sequences in a sample, each one present in two different forms in the population, could generate 1010 different outcomes, permitting the unique identification of human individuals.

The detection of viral or cellular oncogenes is another important field of application of nucleic acid diagnostics. Viral oncogenes (v-oncogenes) are transmitted by retroviruses while their cellular counterparts (c-oncogenes) are already present in normal cells. The cellular oncogenes can, however, be activated by specific modifications such as point mutations (as in the c-K-ras oncogene in bladder carcinoma and in colorectal tumors), small deletions and small insertions. Each of the activation processes leads, in conjunction with additional degenerative processes, to an increased and uncontrolled cell growth. In addition, point mutations, small deletions or insertions may also inactivate the so-called “recessive oncogenes” and thereby leads to the formation of a tumor (as in the retinoblastoma (Rb) gene and the osteosarcoma). The present invention is useful in detecting or identifying the point mutations, small deletions and small mutations that activate oncogenes or inactivate recessive oncogenes, which in turn, cause cancers.

The present invention may also be useful in transplantation analyses. The rejection reaction of transplanted tissue is decisively controlled by a specific class of histocompatibility antigens (HLA). They are expressed on the surface of antigen-presenting blood cells, e.g., macrophages. The complex between the HLA and the foreign antigen is recognized by T-helper cells through corresponding T-cell receptors on the cell surface. The interaction between HLA, antigen and T-cell receptor triggers a complex defense reaction which leads to a cascade-like immune response on the body.

The recognition of different foreign antigens is mediated by variable, antigen-specific regions of the T-cell receptor-analogous to the antibody reaction. In a graft rejection, the T-cells expressing a specific T-cell receptor that fits to the foreign antigen, could therefore be eliminated from the T-cell pool. Such analyses are possible by the identification of antigen-specific variable DNA sequences that are amplified by PCR and hence selectively increased. The specific amplification reaction permits the single cell-specific identification of a specific T-cell receptor.

Similar analyses are presently performed for the identification of auto-immune disease like juvenile diabetes, arteriosclerosis, multiple sclerosis, rheumatoid arthritis, or encephalomyelitis.

The present invention is useful for determining gene variations in T-cell receptor genes encoding variable, antigen-specific regions that are involved in the recognition of various foreign antigens. Thus, the present invention may be useful in predicting the probability of a rejection reaction of transplanted tissue.

The present invention is also useful in genome diagnostics. Four percent of all newborns are born with genetic defects; of the 3,500 hereditary diseases described which are caused by the modification of only a single gene, the primary molecular defects are only known for about 400 of them.

Hereditary diseases have long since been diagnosed by phenotypic analyses (anamneses, e.g., deficiency of blood: thalassemias), chromosome analyses (karyotype, e.g., mongolism: trisomy 21) or gene product analyses (modified proteins, e.g., phenylketonuria: deficiency of the phenylalanine hydroxylase enzyme resulting in enhanced levels of phenylpyruvic acid). The additional use of nucleic acid detection methods considerably increases the range of genome diagnostics.

In the case of certain genetic diseases, the modification of just one of the two alleles is sufficient for disease (dominantly transmitted monogenic defects); in many cases, both alleles must be modified (recessively transmitted monogenic defects). In a third type of genetic defect, the outbreak of the disease is not only determined by the gene modification but also by factors such as eating habits (in the case of diabetes or arteriosclerosis) or the lifestyle (in the case of cancer). Very frequently, these diseases occur in advanced age. Diseases such as schizophrenia, manic depression or epilepsy should also be mentioned in this context; it is under investigation if the outbreak of the disease in these cases is dependent upon environmental factors as well as on the modification of several genes in different chromosome locations.

Using direct and indirect DNA analysis, the diagnosis of a series of genetic diseases has become possible: bladder carcinoma, colorectal tumors, sickle-cell anemia, thalassemias, al-antitrypsin deficiency, Lesch-Nyhan syndrome, cystic fibrosis/mucoviscidosis, Duchenne/Becker muscular dystrophy, Alzheimer's disease, X-chromosome-dependent mental deficiency, and Huntington's chorea, phenylketonuria, galactosemia, Wilson's disease, hemochromatosis, severe combined immunodeficiency, alpha-1-antitrypsin deficiency, albinism, alkaptonuria, lysosomal storage diseases, Ehlers-Danlos syndrome, hemophilia, glucose-6-phosphate dehydrogenase disorder, agammaglobulimenia, diabetes insipidus, Wiskott-Aldrich syndrome, Fabry's disease, fragile X-syndrome, familial hypercholesterolemia, polycystic kidney disease, hereditary spherocytosis, Marfan's syndrome, von Willebrand's disease, neurofibromatosis, tuberous sclerosis, hereditary hemorrhagic telangiectasia, familial colonic polyposis, Ehlers-Danlos syndrome, myotonic dystrophy, osteogenesis imperfecta, acute intermittent porphyria, and von Hippel-Lindau disease. The present invention is useful in diagnosis of any genetic diseases that are caused by point mutations, small deletions or small insertions at defined positions.

In a related aspect, the present invention may be used in testing disease susceptibility. Certain gene variations, although they do not directly cause diseases, are associated to the diseases. In other words, the possession of the gene variations by a subject renders the subject susceptible to the diseases. The detection of such gene variations using the present methods enables the identification of the subjects that are susceptible to certain diseases and subsequent performance of preventive measures.

The present invention is also applicable to pharmocogenomics. For instance, it may be used to detect or identify genes that involve in drug tolerance, such as various alleles of cytochrome P450 gene.

In addition, the present invention provides methods useful for detecting or characterizing residual diseases. In other words, the present methods may be used for detecting or identifying remaining mutant genotypes as in cancer after certain treatments, such as surgery of chemotherapy. It may also useful in identifying emerging mutants, such as genetic variations in certain genes that render a pathogenic organism drug resistant.

E. Use of Nucleic Acid Amplification Methods and Compositions in Pre-mRNA Alternative Splicing Analysis

The methods and compositions for exponential nucleic acid amplification may also be used for performing pre-mRNA alternative splicing analysis. A target cDNA or its portion that is suspected to contain a junction between an upstream exon (Exon A) and a downstream exon (Exon B) is first incorporated into an initial nucleic acid molecule (N1) to be used as a template in a first amplification reaction. The initial nucleic acid molecule also comprises at least one strand of a first nicking agent recognition sequence and thus allows for the first amplification reaction in the presence of a DNA polymerase and a nicking agent that recognizes the first nicking agent recognition sequence. The product (A1) from the first amplification reaction comprises the portion of the target suspected to contain the specific exon-exon junction or its complementary portion. The A1 is then mixed with another template nucleic acid (T2). The T2 molecule comprises, from 5′ to 3′: (i) a first sequence comprising: (a) a 3′ portion of the sense strand of Exon A linked at the 3′ terminus of the 3′ portion to a 5′ portion of the sense strand of Exon B at the 5′ terminus of the 5′ portion, or (b) a 5′ portion of the antisense strand of Exon A linked at the 5′ terminus of the 5′ portion to a 3′ portion of the antisense strand of Exon B at the 3′ terminus of the 3′ portion, wherein if the cDNA contains the junction between Exon A and Exon B, the first sequence of the T2 is at least substantially complementary to the A1 molecule, but if the cDNA does not contain the junction between Exon A and Exon B, the T2 is not substantially complementary to the A1 molecule; (ii) a sequence of the antisense strand of a second NARS; and (iii) a second sequence. An amplification reaction is then performed that amplify another single-stranded nucleic acid molecule (A2) using at least a portion of the second sequence of the T2 molecule as a template if the junction between Exon A and Exon B is present in the target cDNA molecule. The reaction mixture is then analyzed to determine whether any A2 molecules have been amplified. The presence of the A2 molecules in the mixture indicates that the target nucleic acid contains the junction between Exon A and Exon B, whereas the absence of the A2 molecules indicates that the target nucleic acid does not contain the junction between Exon A and Exon B.

1. Definitions

An “exon” refers to any segment of an interrupted gene that is represented in the mature RNA product. An “intron” refers to a segment of DNA that is transcribed, but removed from within the transcript by splicing together the sequences (exons) on either side of it.

A “sense strand” of a cDNA molecule refers to the strand that has an identical sequence as the mRNA molecule from which the cDNA molecule is derived except that the nucleotide “U” in the mRNA is substituted by the nucleotide “T” in the cDNA molecule. An “antisense strand” of a cDNA molecule, on the other hand, refers to the strand that is complementary to the mRNA molecule from which the cDNA molecule is derived.

A “3′ portion” of a strand of an exon refers to a portion of the strand of the exon that comprises the 3′ terminus of the strand of the exon. Likewise, a “5′ portion” of a strand of an exon refers to a portion of the strand of the exon that comprises the 5′ terminus of the strand of the exon.

An exon (Exon A) is “upstream” to another exon (Exon B) in a same gene when the sequence of the sense strand of Exon A is 5′ to the sequence of the sense strand of Exon B. Exon A and Exon B may be further referred to as an upstream exon and a downstream exon, respectively.

A target cDNA molecule refers to a cDNA molecule that is derived from a gene of interest. In other words, it is the product of reverse transcription of an mRNA molecule resulting from the transcription of the gene of interest. The target cDNA molecule may have a partial sequence (i.e., reverse transcribed from a partial mRNA molecule), but preferably a full-length sequence.

A nucleic acid fragment encompassing a first ODNP and a second ODNP refers to a double-stranded nucleic acid fragment that one strand consists of the sequence of the first ODNP, the complementary sequence of the second ODNP, and the sequence between the first ODNP and the complementary sequence of the second ODNP; while the other strand consists of the complementary sequence of the first ODNP, the sequence of the second ODNP, and the sequence between the complementary sequence of the first ODNP and the sequence of the second ODNP.

“Differential splicing” or “alternative splicing” is the production of at least two different mRNA molecules from a same transcript of a gene. For instance, a particular segment of the transcript may be present in one of the mRNA molecules, but be spliced out from other mRNA molecules.

A “location suspected to be the junction of two specific exons” or a “location of a suspected junction of two specific exons” refers to the 3′ terminus of the sense strand of the relatively upstream exon and/or the 5′ terminus of the antisense strand of that exon.

A “junction of Exon A and Exon B” in a target cDNA refers to the location in the sense strand of the target cDNA where the 3′ terminus of Exon A is joined with the 5′ terminus of Exon B and/or the location in the antisense strand of the target cDNA where the 5′ terminus of Exon A is joined with the 3′ terminus of Exon B.

2. Initial Nucleic Acid Molecules (N1)

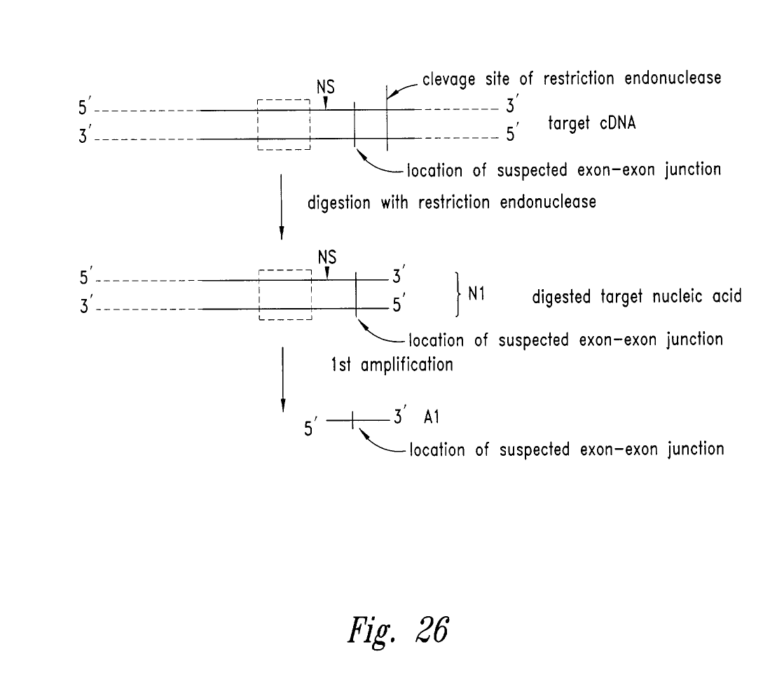

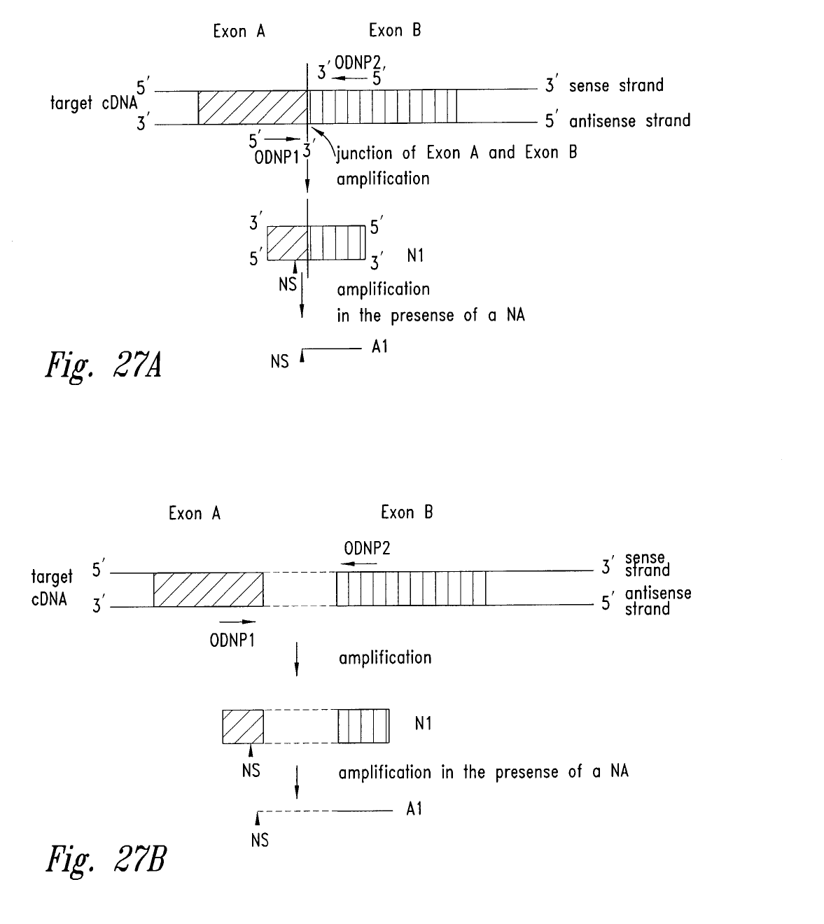

Initial nucleic acid molecules useful for differential splicing analysis may be provided by various approaches. For instance, N1 may be directly derived from a double-stranded target cDNA (e.g., by digestion of the target cDNA with a restriction endonuclease as shown in FIG. 26). Alternatively, N1 may also be prepared by the use of appropriate oligonucleotide primer pairs (e.g., FIGS. 27-30). Several exemplary means for providing initial nucleic acid molecules N1 are described below.

a. First Type of Exemplary Methods for Providing N1 Molecules

In certain embodiments of the present invention, N1 is directly derived from a target cDNA that contains a location suspected to be a specific exon-exon junction and further comprises a nicking agent recognition sequence and a restriction endonucelase recognition sequence. An embodiment with a recognition sequence recognizable by a nicking endonuclease that nicks outside its recognition sequence (e.g., N.BstNB I) as an exemplary nicking agent recognition sequence is illustrated in FIG. 26. As shown in this figure, a target cDNA may be digested by a restriction endonuclease that recognizes a sequence in the target nucleic acid. The digestion product that contains the nicking endonuclease recognition sequence may function as an initial nucleic acid molecule (N1) to amplify a single-stranded nucleic acid fragment (A1). The location suspected to be a specific exon-exon junction needs to be between the nicking site produced by the nicking agent and the cleavage site of the restriction endonuclease so that the location is transferred or incorporated into the amplified A1 fragment.

b. Second Type of Exemplary Methods for Providing N1 Molecules

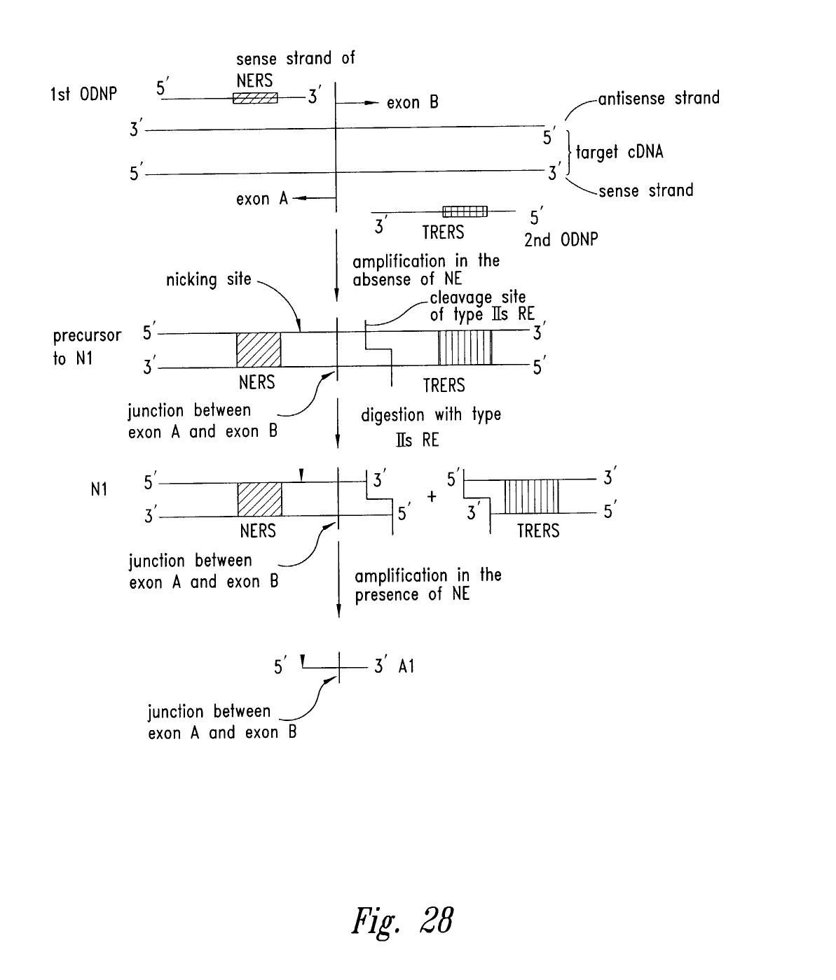

In certain embodiments, an initial nucleic acid molecule N1 is a completely or partially double-stranded nucleic acid molecule produced using various primer pairs. The following section first describes a general method for providing the above initial nucleic acid molecule (FIG. 27) and then provides certain specific embodiments of the general method (FIGS. 28-30).

For determining the presence or absence of a junction of an upstream exon (Exon A) and a downstream exon (Exon B), a primer pair composed of the following two primers may be used: (1) a first primer that comprises a sequence complementary to a portion of the antisense strand of Exon A near the 5′ terminus of Exon A in the antisense strand, and (2) a second primer that comprises a sequence complementary to a portion of the sense strand of Exon B near the 5′ terminus of Exon B in the sense strand (FIG. 27). The complementarity between the first ODNP and the portion of the antisense strand of Exon A needs not be exact, but must be sufficient to allow the ODNP to specifically anneal to that portion of Exon A. Likewise, the complementarity between the second ODNP and the portion of the sense strand of Exon B needs not be exact, but must be sufficient to allow the ODNP to specifically anneal to that portion of Exon B. A portion of a strand of an exon is near one of the termini of the exon if that portion is within 100, 90, 80, 70, 60, 50, 40, 35, 30, 25, 20, 15, or 10 nucleotides from that terminus in that strand. Such a spacing arrangement between the two ODNPs of the ODNP pair enables the amplification of a relatively short fragment encompassing the first and second primers using the target cDNA as a template if the junction of Exon A and Exon B is present in the target cDNA.

Besides the sequence complementarity between each primer and one strand of its corresponding exon, either the first or the second primer must further comprise a sequence of a sense strand of a nicking agent recognition sequence. The recognition sequencer may be recognizable by a nicking endonuclease or a restriction endonuclease. In certain preferred embodiments, both the first and second primers comprise a nicking agent recognition sequence. The presence of the recognition sequence allows the amplified nucleic acid fragments encompassing the first and second primers to function as a template nucleic acid for amplifying a single-stranded nucleic acid fragment (A1) in the presence of a DNA polymerase and a nicking agents that recognizes the recognition sequence.

When the primers and the target cDNA are combined in an amplification reaction, the presence (or absence) and composition of an amplification product reflects the presence or absence of the junction of Exon A and Exon B. If only Exon A or only Exon B is present in the target cDNA, no amplification product will be made using the above primers as primers and the target cDNA as a template. If both Exon A and Exon B are present in the target cDNA, an amplification product (i.e., a N1 molecule or a precursor to N1) will be made that encompasses the first and second primers. If the junction of Exon A and Exon B is present in the target cDNA, the amplification product will contain this junction (FIG. 27A). If the junction of Exon A and Exon B is absent (i.e., there is a sequence between Exon A and Exon B), the amplification product will not contain the junction but contain the sequence between the two exons (FIG. 27B). Thus, characterizing a single-stranded nucleic acid molecule (A1) amplified using N1 as a template and/or another single-stranded nucleic acid molecule (A2) using A1 as a template will indicate whether the target cDNA contains the junction of Exon A and Exon B.

A specific embodiment of the above general method is illustrated in FIG. 28. As indicated in this figure, the first primer comprises a sequence of the sense strand of a nicking endonuclease recognition sequence and anneals to a portion of the antisense strand of Exon A, whereas the second primer comprises a sequence of one strand of a type IIs restriction endonuclease recognition sequence and anneals to a portion of the sense strand of Exon B. When these two primers are used as primers to amplify a portion of the target cDNA, the amplification product (i.e., a precursor to N1) contains both strands of the nicking endonuclease recognition sequence and both strands of the type IIs restriction endonuclease recognition sequence, in addition, the amplification product also contains the junction of Exon A and Exon B if the junction is present in the target cDNA. In the presence of a type IIs restriction endonuclease that recognizes the type IIs restriction endonuclease recognition sequence, the amplification product is digested to produce a partially double-stranded nucleic acid molecule N1 that comprises both strands of the nicking endonuclease recognition sequence and also contains the junction of Exon A and Exon B if the junction is present in the target cDNA.

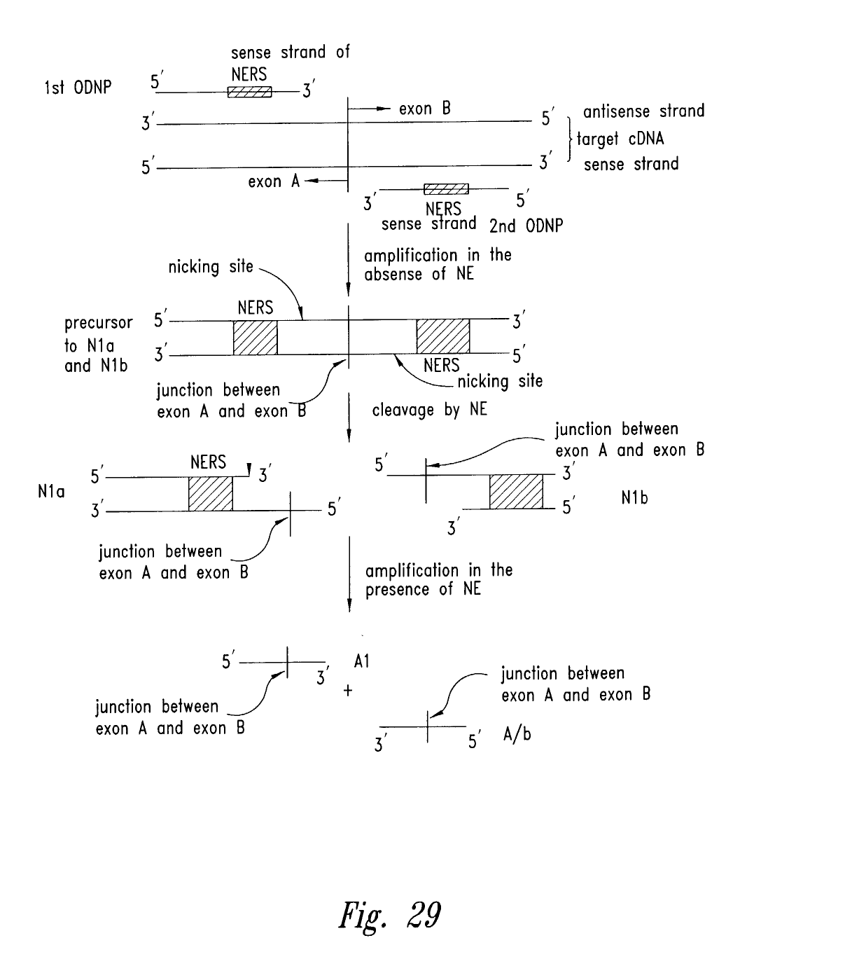

Another specific embodiment of the above general method is illustrated in FIG. 29. As indicated in this figure, both primers comprise a nicking endonuclease recognition sequence. In addition, the first primer is designed to anneal to a portion of the antisense strand of Exon A, whereas the second primer is designed to anneal to a portion of the sense strand of Exon B. When these two primers are used as primers to amplify a portion of the target cDNA, the amplification product (i.e., a precursor to N1) contains the junction of Exon A and Exon B if the junction is present in the target cDNA, as well as two double-stranded nicking endonuclease recognition sequences. These two recognition sequences may or may not be identical to each other, but preferably, they are identical. In the presence of a nicking endonuclease or nicking endonucleases that recognize the recognition sequences, the amplification product is nicked twice (once on each strand) to produce two partially double-stranded nucleic acid molecules (N1a and N1b) that each comprises one of the nicking endonuclease recognition sequences. In addition, the overhang of each of these two molecules also contains the junction of Exon A and Exon B if the junction is present in the target cDNA.

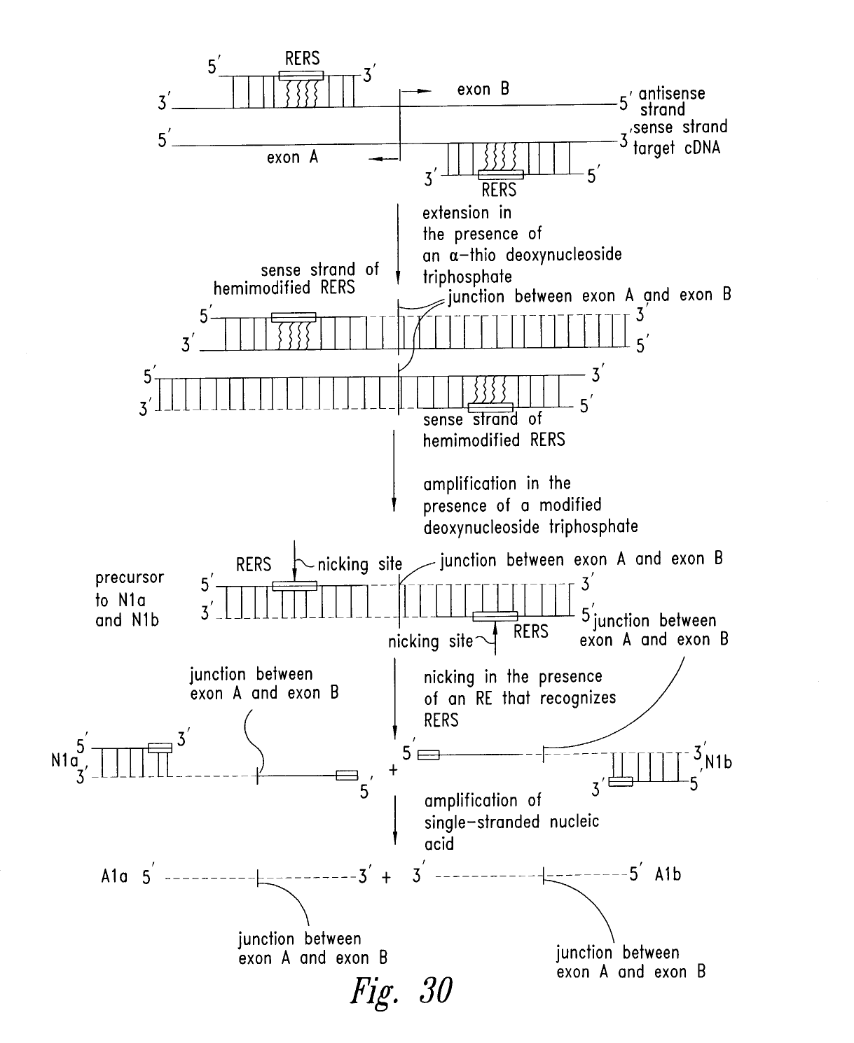

An additional specific embodiment of the above general method is illustrated in FIG. 30. As indicated in this figure, both primers comprise a restriction endonuclease recognition sequence. In addition, the first primer is designed to anneal to a portion of the antisense strand of Exon A, whereas the second primer is designed to anneal to a portion of the sense strand of Exon B. When these two primers are used as primers to amplify a portion of the target cDNA in the presence of a modified deoxynucleoside triphosphate, the amplification product (i.e., a precursor to N1) contains the junction of Exon A and Exon B if the junction is present in the target cDNA, as well as two hemimodified restriction endonuclease recognition sequences. These two hemimodified recognition sequences may or may not be identical to each other, but preferably, they are identical. In the presence of a restriction endonuclease or restriction endonucleases that recognize the recognition sequences, the amplification product is nicked twice (once on each strand) to produce two partially double-stranded nucleic acid molecules (N1a and N1b) that each comprises a sequence of one strand of one of the hemimodified recognition sequences. In addition, the overhang of each of these two molecules also contains the junction of Exon A and Exon B if the junction is present in the target cDNA.

The above first ODNP, the second ODNP or both may be immobilized to a solid support in certain embodiments. In other embodiments, the target cDNA molecule is immobilized.

3. A1 Molecules

As described above, an A1 molecule is amplified using a portion of N1 as a template. This portion of N1 comprises the location suspected to be a specific exon-exon junction so that this location is transferred or incorporated into A1. In certain embodiments, the length of A1 may be regulated to be relatively short in the case where the specific exon-exon junction is present in the target cDNA. For instance, for the second type of providing N1 molecules (FIGS. 27-30), the ODNP pair may be designed to be close to each other when they anneal to the target cDNA. More specifically, the first primer may be designed to anneal to a portion of the antisense strand of the target cDNA close to the 5′ terminus of Exon A, whereas the second primer may be designed to anneal to a portion of the sense strand of the target cDNA close to the 5′ terminus of Exon B. Similar to the diagnostic uses and genetic variation detection of the present invention described above, the short length of an A1 molecule increases amplification efficiencies and rates, allows for the use of a DNA polymerase that does not have a stand displacement activity, and facilitates the detection of A1 molecules via certain technologies such as mass spectrometric analysis. In addition, the short distance between the 3′ terminus of the portion of Exon B to which the second ODNP anneals and the 3′ terminus of the sense strand of Exon B prevents an A1 molecule from hybridizing to a T2 molecule when there exists an extra sequence in the target nucleic acid between Exon A and Exon B.

4. T2 Molecules

As describe above, a T2 molecule suitable for pre-mRNA alternative splicing analysis comprises a sequence of the antisense strand of a nicking agent recognition sequence as well as a sequence, located 3′ to the sequence of the sense strand of the recognition sequence, that is at least substantially complementary to a single-stranded nucleic acid molecule (A1) amplified using a portion of an initial nucleic acid molecule N1 as a template if the target cDNA from which the initial nucleic acid is derived comprises an exon-exon junction of interest (e.g., an upstream exon—Exon A, and a downstream exon—Exon B).

More specifically, a T2 molecule comprises, from 5′ to 3′: (i) a first sequence comprising a portion of the target nucleic acid that comprises (a) a 3′ portion of the sense strand of Exon A linked at the 3′ terminus of the 3′ portion to a 5′ portion of the sense strand of Exon B at the 5′ terminus of the 5′ portion, or (b) a 5′ portion of the antisense strand of Exon A linked at the 5′ terminus of the 5′ portion to a 3′ portion of the antisense strand of Exon B at the 3′ terminus of the 3′ portion, wherein if the cDNA contains the junction between Exon A and Exon B, the first sequence of the T2 is at least substantially complementary to the A1 molecule, but if the cDNA does not contain the junction between Exon A and Exon B, the T2 is not substantially complementary to the A1 molecule; (ii) a sequence of the antisense strand of a second NARS; and (iii) a second sequence.

To ensure that a T2 molecule is not substantially complementary to an A1 molecule amplified when a target cDNA does not contain the exon-exon junction, the 3′ portion of the sense strand of Exon A or the 3′ portion of the antisense strand of Exon B in the first sequence of the T2 molecule cannot be so long that the 3′ portion alone (i.e., without the remaining portion of the first sequence) is able to anneal to an A1 molecule that comprises the complement of the 3′ portion. Otherwise, the present method will not be able to distinguish the situation where the target cDNA has the specific exon-exon junction from that where the target cDNA has an extra sequence between the upstream exon and the downstream exon. In other words, an A1 molecule amplified using a portion of the target cDNA that has an extra sequence between the upstream exon and the downstream exon would still be able to anneal to the T2 molecule that has a long 3′ portion of the sense strand of the upstream exon or a long 3′ portion of the antisense strand of the downstream exon to initiate the amplification of another molecule (A2) using a portion of the T2 molecule as a template.

In certain embodiments, a T2 molecule comprises a second sequence located 5′ to a sequence of the antisense strand of a NARS that is either substantially or completely identical to a first sequence located 3′ to the sequence of the antisense strand of the NARS.

A T2 molecule may be immobilized to a solid support, preferably via its 5′ terminus, in certain embodiments. In other embodiments, a T2 molecule may not be immobilized.

5. Characterizing Amplified Single-Stranded Nucleic Acids

Whether a target cDNA molecule has a specific exon-exon junction may be detected or identified by characterizing an amplification product (i.e., A1 or A2). Any method suitable for characterizing single-stranded nucleic acid molecules may be used. Exemplary techniques include, without limitation, chromatography such as liquid chromatography, mass spectrometry and electrophoresis. Detailed description of various exemplary methods may be found in U.S. Prov. Appl. Nos. 60/305,637 and 60/345,445.

The characteristics of the amplified single-stranded nucleic acid fragments (e.g., the mass to charge ratio obtained by mass spectrometric analysis) are subsequently compared with those of single-stranded nucleic acid fragments predicted in view of the positions and compositions of the primers used in preparing template nucleic acid fragments and with the assumption that the junction between the two exons to which the primers are complementary is present. If the characteristics of the amplified and the predicted nucleic acid fragments are identical, the particular exon-exon junction that was assumed to be present in the target cDNA molecule is in fact present in that target cDNA molecule. The prediction of the sequence and the characteristics (e.g., mass to charge ratio) of the single-stranded nucleic acid fragment that would be amplified is based on the knowledge about consensus sequences near exon-intron junctions. This knowledge allows one of ordinary skill in the art to pinpoint the exon-intron junctions and thus predicts the exact locations of exon-exon junctions when the intron between the two exons has been spliced out.

6. Compositions and Kits Useful in Pre-mRNA Differential Splicing Analysis

Compositions and kits useful in pre-mRNA differential splicing analysis may be the same as those described above for exponential nucleic acid amplification. In certain embodiments, these kits may further comprise one or more additional components useful in characterizing amplification products. For instance, the additional component may be (1) a chromatography column; (2) a buffer for performing chromatographic characterization or separation of nucleic acids; (3) microtiter plates or microwell plates; (4) oligonucleotide standards (e.g., 6 mer, 7 mer, 8 mer, 10 mer, 12 mer, 14 mer and 16 mer) for liquid chromatography and/or mass spectrometry; (5) a reverse transcriptase; (6) a buffer for a reverse transcriptase, and (7) an instruction booklet for using the kits.

7. Applications of the Present Pre-mRNA Differential Splicing Analysis

The present invention is useful in detecting any mRNA differential splicing of interest. Alternative pre-mRNA splicing is an important mechanism for regulating gene expression in higher eukaryotes. By recent estimates, the primary transcripts of ˜30% of human genes are subject to alternative splicing, often regulated in specific spatial/temporal patterns during normal development. In complex genes alternative splicing can generate dozens or even hundreds of different mRNA isoforms from a single transcript (Breitbart and Nadal-Ginard, Annu. Rev. Biochem. 56: 467-95,1987; Missler and Sudhof, Trends Genet 14: 20-6,1998; Gascard et al., Blood 92:4404-14,1998). In many cases the alternatively spliced exon encodes a protein domain that is functionally important for catalytic activity or binding interactions, the resulting proteins can exhibit different or even antagonistic activities.

As discussed in detail herein above, the present invention provides methods, compositions, and kits for detecting pre-mRNA alternative splicing, including the detection of alternative splicing at a terminus of a particular exon of a gene in a cDNA molecule or a cDNA population, and at every terminus of every exon of a gene in a cDNA molecule or a cDNA population. Due to the importance of pre-mRNA splicing, these methods, compositions and kits will find utility in a wide variety of applications such as disease diagnosis, predisposition, and treatment, crop cultivation and animal breeding, development regulations of plants and animals, drug development and manipulation of responses of an organism to external stimuli (e.g., extreme temperatures, poison, and light).

For instance, the present method may be used to identify and/or characterize pre-mRNA splicing patterns unique to a pathological condition. Abnormal pre-mRNA splicings in many genes have been implicated in various diseases or disorders, especially in cancers. In small cell lung carcinoma, the gene of protein p130, which belongs to the retinoblastoma protein family is mutated at a consensus splicing site. This mutation results in the removal of exon 2 and the absence of synthesis of the protein due to the presence of a premature stop codon. Likewise, in certain non small cell lung cancers, the gene of protein p161 NK4A, which is an inhibitor of cyclin dependant kinase cdk4 and cdk6, is mutated at a donor splicing site. This mutation results in the production of a truncated short half-life protein. In addition, WT1, the Wilm's tumor suppressor gene, is transcribed into several messenger RNAs generated by alternative splicings. In breast cancers, the relative proportions of different variants are modified in comparison to healthy tissue, hence yielding diagnostic tools or insights into understanding the importance of the various functional domains of WT1 in tumoral progression. A similar alteration process affecting ratios among different mRNA forms and protein isoforms during cell transformation is also found in neurofibrin NF1. Moreover, in head and neck cancer, one of the mechanisms by which p53 is inactivated involved a mutation at a consensus splicing site. Furthermore, an altered splicing pattern of the IRF-1 tumor suppressor gene transcript results in the inactivation of the tumor suppressor and an acceleration of exon skipping in IRF-1 mRNA is indicative of a number of hematopoietic disorders including overt leukemia from myelodysplastic syndrome, acute myeloid leukemia, and the myelodysplastic syndromes (U.S. Pat. No. 5,643,729).

The present method may be used to compare the splicing pattern of the transcript of a gene that is known or suspected to be associated with a disease (or disorder) condition, and to identify exons of which presence or absence is unique to the disease (or disorder) condition or to identify the alteration in the ratio among different splicing variants unique to the disease (or disorder) condition. The identification of the exons that are absence in a disease (or disorder) condition may indicate that the domains encoded by the exons are important to the normal functions of healthy cells and that the signaling pathways involving such domains may be restored for therapeutic purposes. On the other hand, the identification of the exons uniquely present in a disease (or disorder) condition may be used as diagnostic tools and the domains encoded thereof be considered as screening targets for compounds of low molecular weight intended to antagonize signal transduction mediated by the domains. In addition, the antibodies with specific affinities to these domains may also be used as diagnostic tools for the disease (or disorder) condition.

The present method may also be used to identify and/or characterize the pre-mRNA differential splicing important in organism development. Alternative splicing plays a major role in sex determination in Drosophilia, antibody response in humans and other tissue or developmental stage specific processes (Chabot, Trends Genet. 12: 472-8; Smith et al., Annu. Rev. Genet. 23: 527-77, 1989; Breitbart et al., Cell 49: 793-803, 1987). Thus, the present method may be used to compare pre-mRNA splicing patterns of a gene that is known or suspected to be involved in development regulation at different developmental stages. The identification and/or characterization of the presence of differential splicing in the gene may provide guidance in regulating the corresponding development process to obtain desirable traits (e.g., bigger fruits, higher protein or oil content seeds, higher milk production).

The present method may also be used to identify and/or characterize the pre-mRNA differential splicing important in organisms' responses to various external stimuli. The pre-mRNA splicing pattern of a gene that is known or suspected to play a role in response to a particular stimulus (e.g., pathogen attack) of an untreated organism may be compared with that of an organism subjected to the stimulus. The identification and/or characterization of the splicing pattern unique to the organism subjected to the stimulus may provide guidance in manipulating the corresponding response process to enhance (if the response is desirable) or to reduce/eliminate (if the response is undesirable) the response.

The following examples are provided by way of illustration and not limitation.

### EXAMPLES

### Example 1

### Exponential Amplification of a Nucleic Acid Sequence

This example describes the exponential amplification of a specific nucleic acid sequence using a nicking restriction endonuclease and DNA polymerase.

The oligonucleotides used in this example were obtained from MWG Biotech (North Carolina) and their sequences are listed below with the sequence of the sense or the antisense strand of the N.BstNB I recognition sequence underlined:

Template No. 1 (T1):
3′-acaaggtcagcatccactcagacaaggtcagcatcca-5′
Template No. 2 (T2):
3′-acaaggtcagcatccactcagctacaaggtcagcatcca-5′
Trigger ODNP:
5′-tgttccagtcgtaggtgagtctgtt-3′

The following reaction mixture was assembled at room temperature:

75 ul water

10 ul 10×Thermopol buffer (from NEB (Beverly, Mass.))

5 ul 10×N.BstNBI (from NEB)

5 ul T1 at 0.2 nanomoles/ul

5 ul TOP1 at 0.2 nanomoles/ul The mixture was heated to 95° C. and then cooled to 50° C. and held at 50° C. for 10 minutes. After the incubation at 50° C., the following duplex (N1) was formed:

5′-tgttccagtcgtaggtgagtctgtt-3′
3′-acaaggtcagcatccactcagacaaggtcagcatcca-5′

The above mixture was diluted into a reaction mixture containing the following:

25 ul 10×Thermopol buffer (from NEB)

12.5 ul 10×N.BstNBI (from NEB)

0.5 ul of the duplex mixture described above

10 ul 25 mM dNTPs (from NEB)

100 ul 1 M trehalose (from Sigma (St. Louis, Mo.))

25 units N.BstNBI nicking enzyme (from NEB)

5 units exo⁻ Vent DNA polymerase (from NEB)

5 ul T2

102 ul water

The reaction was incubated at 60° C. for 15 minutes. After 15 minutes, 10 ul of the reaction was sampled and subjected to mass spectrometry.

During the incubation at 60°, the following duplex (H1) was filled in by the action of the DNA polymerase with “▾” indicating the nicking site of N.BstNB I:

▾
5′-tgttccagtcgtaggtgagtctgttccagtcgtaggt-3′
3′-acaaggtcagcatccactcagacaaggtcagcatcca-5′

The nicking enzyme cuts the upper strand of H1 and releases the fragment has the sequence 5′-ccagtcgtaggt-3′ (referred to as “A1”). As this fragment (i.e., A1) is made, the following duplex (N2) is formed in the 60° C. reaction mixture.

5′-ccagtcgtaggt-3′
3′-acaaggtcaccatccactcagctacaaggtcagcatcca-5′

The polymerase fills in the duplex to form the following fragment (H2):

```
                                                          ▾
        5′-ccagtcgtaggtgagtcgatgttccagtcgtaggt-3′
3′-acaaggtcaccatccactcagctacaaggtcagcatcca-5′
```

The N.BstNB I nicks the duplex and generate the fragment have the sequence 5′-ttccagtcgtaggt-3′ (referred to as “A2”), which can prime T2 to form the following partial double-stranded fragment:

5′-ttccagtcgtaggt-3′
3′-acaaggtcaccatccactcagctacaaggtcagcatcca-5′

The above partial double-stranded fragment is filled in by the DNA polymerase to form the following duplex:

```
                                                              ▾
    5′-ttccagtcgtaggtgagtcgatgttccagtcgtaggt-3′
3′-acaaggtcaccatccactcagctacaaggtcagcatcca-5′
```

This duplex is then nicked by the N.BstNB I, generating the fragment 5′-ttccagtcgtaggt-3′ (i.e., A2). The nicking and extension process is repeated multiple times, resulting in the amplification of A2 molecules.

The amplified fragment A2 has a predicted mass/charge profile as follows:

| Mass/charge value | Mass/charge |
|---|---|
| 4348.8 − 1 = 4347.8 | 1 |
| 2174.9 − 1 = 2173.9 | 2 |
| 1449.9 − 1 = 1448.9 | 3 |
| 1087.5 − 1 = 1086.5 | 4 |

Mass spectrometry analyses of the amplified fragment A2 are shown in FIG. 16. The top panel shows the ion current for a fragment with a mass/charge ratio of 1448.6. The total ion current is 229 units. The middle panel shows the trace from the diode array. The bottom panel shows the total ion current from the mass spectrometer.

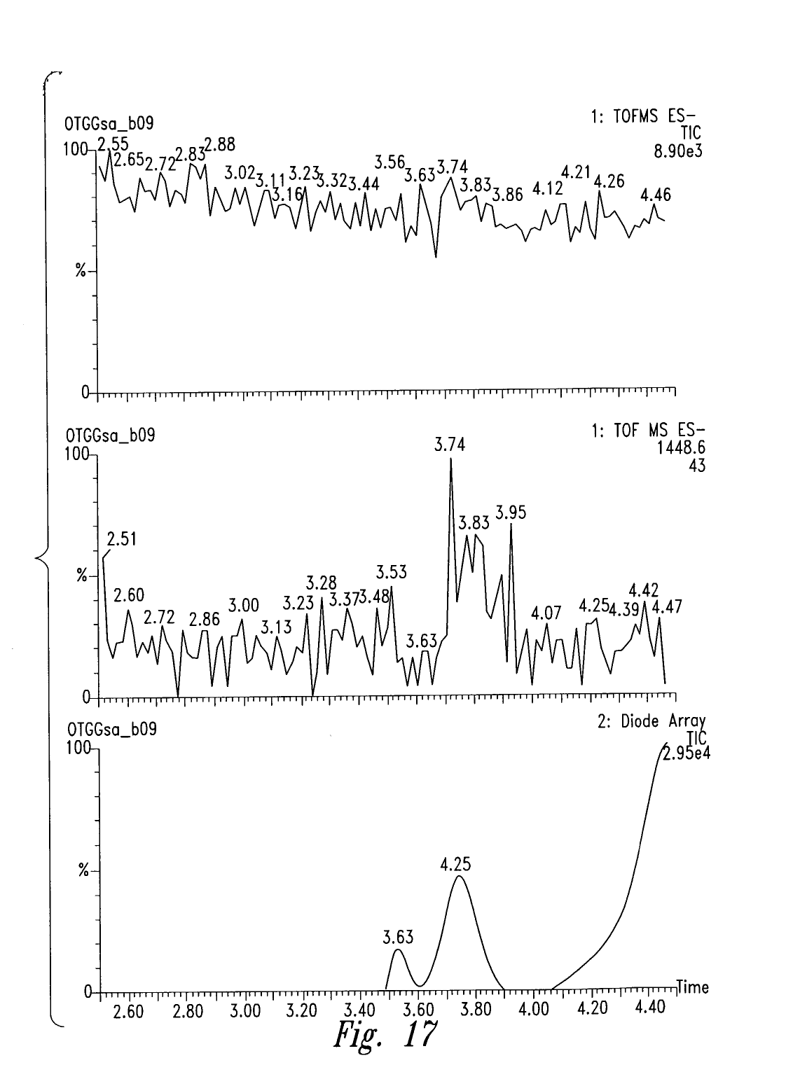

Mass spectrometry analyses in a control experiment are shown in FIG. 17. The top panel shows the total ion current from the mass spectrometer. The middle panel shows the ion current for a fragment with a mass/charge ratio of 1448.6. The total ion current is 43 units, which represents only background. The bottom panel shows the trace of diode array.

The above results indicate that there was exponential amplification of fragment A2 (10⁹ fold amplification was observed) and that no product was made in the control experiment in which TOP1 was omitted.

### Example 2

### Exponential Amplification of a Trigger Oligonucleotides

This example describes exponential amplification of a trigger oligonucleotide using a template oligonucleotide.

The oligonucleotide sequences used in this example are as follows with the sequence of the antisense strand of the recognition sequence of N.BstNB I underlined:

Template (T1):
5′-cctacgactggaacagactcacctacgactgg a-3′
Trigger:
5′-ccagtcgtagg-3′

The above template and trigger form the following duplex when they anneal to each other

| | |
|---|---|
| Trigger: | 5′-ccagtcgtagg-3′ |
| Template: | 3′-aggtcagcatccactcagacaaggtcagcatcc-5′ |

In the presence of a DNA polymerase (e.g., exo- Vent or 9° Nm™), the above duplex is extended from the 3′ end of the trigger oligonucleotide to form the following extension product with the sequences of both strands of the recognition sequence of N.BstNB I underlined:

5′-ccagtcgtaggtgagtctgttccagtcgtagg-3′
3′-aggtcagcatccactcagacaaggtcagcatcc-5′

In the presence of N.BstNB I, the above extension product is nicked and produces a partially double-stranded nucleic acid and a single-stranded nucleic acid fragment (A1) having a sequence identical to that of the trigger oligonucleotide:

5′-ccagtcgtaggtgagtctgtt-3′
+ 5′-ccagtcgtagg-3′
3′-aggtcagcatccactcagacaaggtcagcatcc-5′

The above extension and nicking may be repeated multiple times, resulting amplification of A1 molecules. In addition, A1 molecules may anneals to single-stranded T1 molecules, resulting additional amplification of A1 molecules.

The following reaction mixture was assembled at 4° C.

100 ul 10×Thermopol buffer

50 ul 10×N.BstNBI buffer

16 ul 25 mM dNTPs

0.5 ul T1 at 100 pmol/ul

80 ul 2000 units/ml N.BstNBI (NEB)

24 ul 9° Nm™ DNA polymerase (NEB)

10 ul 400×SYBR (Molecular Probes, Eugene Wash.)

740 ul water

The reaction mixture was thoroughly mixed at 4° C. 150 ul of the reaction mixture placed in a first tube, and 100 ul placed in 9 additional tubes. The trigger was diluted 100 times in water and then 1 ul placed in the first tube. Nine three-fold dilutions were then made.

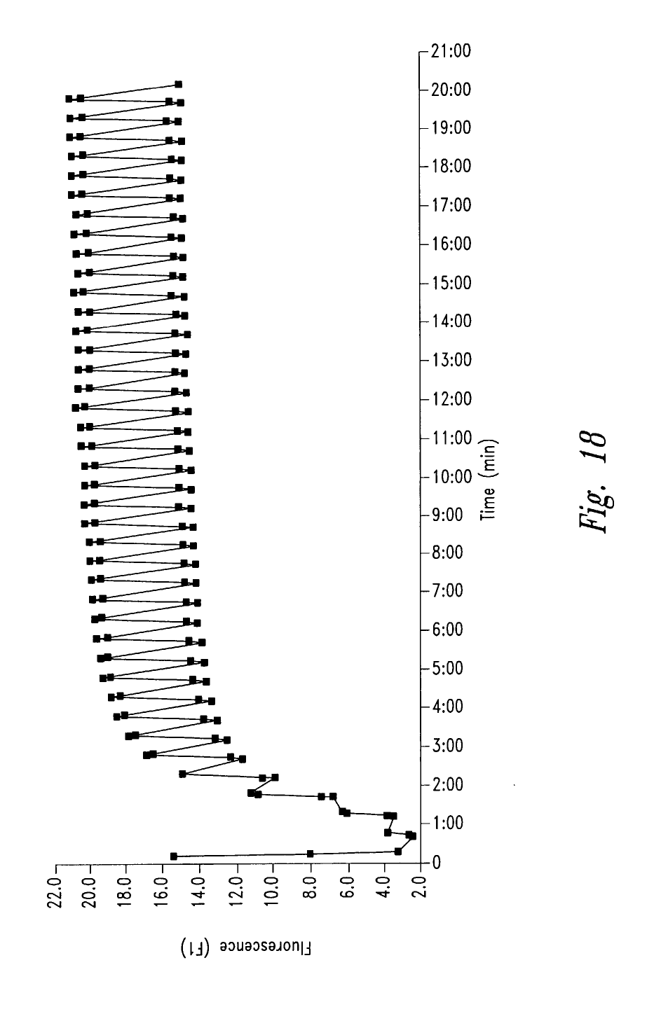

30 ul of each reaction was added to the light cycler capillaries. The capillaries were incubated at 60° C. for the indicated times. A representative result is shown in FIG. 18. This figure shows the accumulation of fluorescence in one of the light cycler capillaries as a function of time. The data are summarized in the following table:

| | Time to Maximum |
|---|---|
| Concentration of Trigger | Fluorescence |
| 3.3 × 10⁻³ picomoles/ul | 5 minutes |
| 1.1 × 10⁻³ picomoles/ul | 7 minutes |
| 3.7 × 10⁻⁴ picomoles/ul | 9 minutes |
| 1.2 × 10⁻⁴ picomoles/ul | 11 minutes |
| 4.1 × 10⁻⁵ picomoles/ul | 17 minutes |
| 1.4 × 10⁻⁵ picomoles/ul | 20 minutes |
| 4.5 × 10⁻⁶ picomoles/ul | 20 minutes |
| 1.5 × 10⁻⁶ picomoles/ul | 20 minutes |
| 5.0 × 10⁻⁷ picomoles/ul | 20 minutes |

The above result shows that there exists an approximate 20,000-fold range over which differences in starting concentrations of a trigger oligonucleotide can be measured and compared.

### Example 3

### Amplification of an Oligonucleotide Using an Immobilized Template

This example describes the amplification of an oligonucleotide using an immobilized template oligonucleotide (T2) as a template. The oligonucleotide was first amplified using a trigger oligonucleotide and a soluble template oligonucleotide (T1). The amplified oligonucleotide in solution then annealed to the immobilized T2. The T2 molecule comprises a sequence of the antisense sequence of the double-stranded recognition sequence of N.BstNB I. It also comprises a first sequence located 5′ to the sequence of the antisense sequence that is exactly identical to the second sequence located 5′ to the sequence of the antisense sequence. In the presence of N.BstNB I and a DNA polymerase, the oligonucleotide was further amplified using a portion of the immobilized T2 as a template.

The sequences of the trigger oligonucleotide, the soluble template oligonucleotide (T1) and the immobilized oligonucleotide (T2) are shown below with the sequences of the sense and antisense strand of the nicking agent recognition sequence underlined. The T2 was immobilized by attaching its 5′ terminus to a PEI-coated tip via a hexyl-amine group:

Tripper oligonucleotide

5′-CCGATCTAGTGAGTCGCTC-3′

The T1 molecule

3′-GGCTAGATCACTCAGCGAGGGTCAGCATCC-5′

The immobilized T2 molecule

5′-amino-CCTACGACTGGAACAGACTCACCTACGACTGGA-3′

In solution, the trigger oligonucleotide and the T1 molecule formed the following duplex:

5′-CCGATCTAGTGAGTCGCTC-3′
3′-GGCTAGATCACTCAGCGAGGGTCAGCATCC-5′

This duplex was present at 0.01 femomoles/50 ul in the solution. When this duplex is filled in by a DNA polymerase, the following duplex was formed:

5′-CCGATCTAGTGAGTCGCTCCCAGTCGTAGG-3′
3′-GGCTAGATCACTCAGCGAGGGTCAGCATCC-5′

In the presence of N.BstNB I, the above duplex was nicked and the following oligonucleotide was released:

5′-CCAGTCGTAGG-3′

The extension and nicking cycle may be repeated multiple times, resulting in the amplification of the above oligonucleotide.

The amplified oligonucleotide annealed to the immobilized template T2 and formed the following duplex:

3′-GGATGCTGACC-5′
5′-amino-CCTACGACTGGAACAGACTCACCTACGACTGGA-3′

The above duplex was extended in the present of the DNA polymerase to form the following duplex:

3′-GGATGCAGTCCTTGTCTGAGTGGATGCTGACC-5′
5′-amino-CCTACGACTGGAACAGACTCACCTACGACTGGA-3′

In the presence of N.BstNB I, the above duplex was nicking and the oligonucleotide having a sequence 3′-GGATGCAGTCC-5′ was released. The extension and nicking cycle may be repeated multiple times, resulting the amplification of the above oligonucleotide. The oligonucleotide was able to annealed to another immobilized template T2 and initiated additional of the oligonucleotide itself.

The amplified oligonucleotide has an m/z value of 1246. About 2 units of the oligonucleotide with an m/z value of 1246 was made in 10 minutes. This corresponds to about 1012 molecules.

From the foregoing it will be appreciated that, although specific embodiments of the invention have been described herein for purposes of illustration, various modifications may be made without deviating from the spirit and scope of the invention. Accordingly, the invention is not limited except as by the appended claims.

All of the above U.S. patents, U.S. patent application publications, U.S. patent applications, foreign patents, foreign patent applications and non-patent publications referred to in this specification and/or listed in the Application Data Sheet, are incorporated herein by reference, in their entirety.

What is claimed is:

1. A method for amplifying a nucleic acid molecule (A2), comprising:
(A) providing an at least partially double-stranded nucleic acid molecule (N1) comprising at least one of
(i) a nucleotide sequence of a sense strand of a first nicking agent recognition sequence (NARS), and
(ii) a nucleotide sequence of an antisense strand of the first NARS;
(B) amplifying a first single-stranded nucleic acid molecule (A1) in the presence of a first nicking agent (NA) that recognizes the first NARS, a DNA polymerase, and one or more deoxynucleoside triphosphate(s), where the amplifying uses a portion of N1 as a template for the polymerase;
(C) providing a second single-stranded nucleic acid molecule (T2) comprising, from 5′ to 3′,
(i) a template nucleotide sequence,
(ii) a sequence of an antisense strand of a second NARS, and
(iii) a sequence that is at least substantially complementary to A1; and
(D) amplifying a third single-stranded nucleic acid molecule (A2) in the presence of T2, A1, the first NA, a second NA that recognizes the second NARS, the DNA polymerase and the deoxynucleoside triphosphate(s), where A2 is complementary to at least a portion of the template nucleotide sequence of T2.

2. The method of claim 1 wherein the first NARS is identical to the second NARS.

3. The method of claim 1 wherein both the first and the second NAs are a nicking endonuclease (NE).

4. The method of claim 1 wherein steps (A)-(D) are performed in a single vessel.

5. The method of claim 1 wherein the first NARS contains at least one mismatched base pair.

6. The method of claim 1 wherein N1 comprises the sequence of the sense strand of the first NERS.

7. The method of claim 1 wherein N1 comprises the sequence of the antisense strand of the first NERS.

8. The method of claim 7 wherein both the first and the second NAs are a restriction endonuclease (RE).

9. The method of claim 1 wherein N1 is provided by annealing a trigger oligonucleotide primer (ODNP) and a single-stranded nucleic acid (T1) comprising the sequence of the sense strand or the antisense strand of the first NERS.

10. The method of claim 1 wherein A2 is at least substantially identical to A1.

11. The method of claim 1 wherein A2 is exactly identical to A1.

12. The method of claim 1 wherein A1 is from 8 to 24 nucleotides in length.

13. The method of claim 12 wherein A1 is from 12 to 17 nucleotides in length.

14. The method of claim 1 wherein A2 is from 8 to 24 nucleotides in length.

15. The method of claim 1 wherein A2 is from 12 to 17 nucleotides in length.

16. The method of claim 1 wherein the initial number of T2 is more than that of T1.

17. The method of claim 1 wherein N1 is derived from a genomic DNA.

18. The method of claim 1 wherein N1 is a portion of a genomic DNA.

19. The method of claim 1 wherein N1 is a partially double-stranded nucleic acid molecule comprising:
(a) a sequence of a sense strand of the first NARS, a sequence of an antisense strand of the first NARS, or both; and
(b) either a 5′ overhang in the strand that either the strand itself or an extension product thereof contains a nicking site (NS) produced by the first NA, or a 3′ overhang in the strand that neither the strand itself nor an extension product thereof contains the NS, wherein each overhang comprises a nucleic acid sequence that is at least substantially complementary to a target nucleic acid;
(c) a sequence within the strand that neither the strand nor the extension product thereof contains the NS, the sequence located at 5′ to the position corresponding to the NS and functioning as a template for amplifying A1.

20. The method of claim 19 wherein the target nucleic acid is one strand of a denatured double-stranded nucleic acid.

21. The method of claim 20 wherein the double-stranded nucleic acid is genomic nucleic acid or cDNA.

22. The method of claim 19 wherein the target nucleic acid is an RNA molecule.

23. The method of claim 19 wherein the target nucleic acid is derived from nucleic acid obtained from a source selected from a bacterium, a virus, a fungus and a parasite.

24. A method for amplifying a nucleic acid molecule (A2), comprising:
(A) forming a mixture comprising:
(i) an at least partially double-stranded nucleic acid molecule (N1) comprising a sequence of an antisense strand of a first nicking agent recognition sequence (NARS);
(ii) a single-stranded nucleic acid molecule (T2) comprising, from 5′ to 3′:
(a) a template nucleotide sequence,
(b) a sequence of an antisense strand of a second NARS, and
(c) a sequence that is at least substantially identical to a portion of N1 located 5′ to the antisense strand of the NARS in N1;
(iii) a first nicking agent (NA) that recognizes the first NARS; a second NA that recognizes the second NARS; a DNA polymerase; and one or more deoxynucleoside triphosphate(s); and
(B) maintaining said mixture at conditions that amplify a single-stranded nucleic acid molecule (A1) using a portion of N1 as a template and further amplify another single-stranded nucleic acid molecule (A2) using the template nucleotide sequence of T2 as a template.

25. The method of claim 24 wherein the first NARS is identical to the second NARS.

26. The method of claim 24 wherein the first NARS contains at least one mismatched base pair.

27. The method of claim 24 wherein both the first and the second NAs are a nicking endonuclease (NE).

28. The method of claim 24 wherein the NA is a restriction endonuclease (RE).

29. The method of claim 24 wherein T1 is substantially identical to T2.

30. The method of claim 24 wherein T1 is exactly identical to T2.

31. The method of claim 24 wherein T1 is not substantially or exactly identical to T2.

32. The method of claim 24 wherein A1 is substantially identical to A2.

33. The method of claim 24 wherein A1 is exactly identical to A2.

34. The method of claim 24 wherein A1 is not substantially or exactly identical to A2.

35. The method of claim 24 wherein the sequence (A)(ii)(c) is exactly identical to a portion of N1 located 5′ to the antisense strand of the first NARS.

36. The method of claim 24 wherein the 3′ terminus of T2 is linked to a phosphate group.

37. The method of claim 24 wherein N1 is provided by annealing a trigger oligonucleotide primer (ODNP) to a single-stranded target nucleic acid (T1) that comprises, from 5′ to 3′:
(A) a sequence of an antisense strand of the first NARS; and
(B) a sequence that is at least substantially complementary to at least a portion of the trigger ODNP.

38. The method of claim 37 wherein A1 is substantially identical to the trigger ODNP.

39. The method of claim 37 wherein A1 is exactly identical to the trigger ODNP.

40. The method of claim 37 wherein A2 is substantially identical to the trigger ODNP.

41. The method of claim 37 wherein A2 is exactly identical to the trigger ODNP.

42. The method of claim 37 wherein the sequence (B) of T1 is exactly complementary to at least a portion of the trigger ODNP.

43. The method of claim 37 wherein the 3′ terminus of T1 is linked to a phosphate group.

44. The method of claim 37 wherein the trigger ODNP is derived from nucleic acid obtained from a source selected from a bacterium, a virus, a fungus and a parasite.

45. The method of claim 24 wherein at least one of the deoxynucleoside triphosphate(s) is labeled.

46. The method of claim 45 wherein the labeled deoxynucleoside triphosphate is a deoxynucleoside triphosphate linked to a label selected from the group consisting of a radiolabel, an enzyme, a fluorescent dye, digoxigenin and biotin.

47. The method of claim 24 further comprising detection of A2.

48. The method of claim 47 wherein the detection is performed at least partially by a technique selected from the group consisting of luminescence spectroscopy or spectrometry, fluorescence spectroscopy or spectrometry, mass spectrometry, liquid chromatography, fluorescence polarization, and electrophoresis.

49. The method of claim 47 wherein the detection is performed in the presence of a fluorescence intercalating agent.

50. The method of claim 24 wherein the mixture further comprises:
(iv) a single-stranded nucleic acid molecule (T3) comprising, from 3′ to 5′:
(a) a sequence that is at least substantially identical to at least a portion of the template nucleotide sequence of T2;
(b) a sequence of an antisense strand of a third NARS; and
(c) a second template nucleotide sequence; and
(C) maintaining said mixture at conditions that amplify a single-stranded nucleic acid molecule (A3) complementary to at least a portion of the second template nucleotide sequence of T3.

51. The method of claim 50 wherein the first, second and third NARSs are identical to each other.

52. The method of claim 50 wherein sequence (a) of T3 is exactly identical to at least a portion of the template nucleotide sequence of T2.

53. A method for amplifying a nucleic acid molecule (A2), comprising
(A) forming a mixture of
(i) an at least partially double-stranded nucleic acid molecule (N1) comprising a sequence of a sense strand of a first nicking endonuclease recognition sequence (NERS);
(ii) a single-stranded nucleic acid molecule (T2) that comprises, from 3′ to 5′:
(a) a sequence that is at least substantially complementary to a portion of N1 located 3′ to the sense strand of the NERS in N1,
(b) a sequence of an antisense strand of a second NERS, and
(c) a template nucleotide sequence;
(iii) a first nicking endonuclease (NE) that recognizes the first NERS; a second NE that recognizes the second NERS; a DNA polymerase; and one or more deoxynucleoside triphosphate(s); and
(B) maintaining said mixture at conditions that amplify a single-stranded nucleic acid molecule (A2) using the template nucleotide sequence of T2 as a template.

54. The method of claim 82 wherein the first NERS is identical to the second NERS.

55. The method of claim 82 wherein sequence (ii) (a) is exactly complementary to a portion of N1 located 3′ to the sense strand of the NERS.

56. The method of claim 82 wherein the 3′ terminus of T2 is linked to a phosphate group.

57. The method of claim 82 wherein N1 is provided by annealing a trigger oligonucleotide primer (ODNP) to a single-stranded target nucleic acid (T1) that comprises, from 5′ to 3′:
(A) a sequence of a sense strand of the first NERS; and
(B) a sequence that is at least substantially complementary to at least a portion of the trigger ODNP.

58. The method of claim 57 wherein sequence (A) is exactly complementary to at least a portion of the trigger ODNP.

59. The method of claim 57 wherein the trigger ODNP is derived from nucleic acid obtained from a source selected from a bacterium, a virus, a fungus and a parasite.

60. The method of claim 53 wherein at least one of the deoxynucleoside triphosphate(s) is labeled.

61. The method of claim 60 wherein the labeled deoxynucleoside triphosphate is a deoxynucleoside triphosphate linked to a label selected from the group consisting of a radiolabel, an enzyme, a fluorescent dye, digoxigenin and biotin.

62. The method of claim 53 further comprising the detection of A2.

63. The method of claim 62 wherein the detection is performed at least partially by a technique selected from the group consisting of luminescence spectroscopy or spectrometry, fluorescence spectroscopy or spectrometry, mass spectrometry, liquid chromatography, fluorescence polarization, and electrophoresis.

64. The method of claim 62 wherein the detection is performed in the presence of a fluorescence-labeled compound that specifically binds to a double-stranded nucleic acid molecule.

65. The method of claim 53 wherein the mixture further comprises
(iv) a single-stranded nucleic acid molecule (T3) that comprises from 3′ to 5′:
(a) a sequence that is at least substantially identical to at least a portion of the template nucleotide sequence of T2;
(b) a sequence of an antisense strand of the NERS; and
(c) a second template nucleotide sequence; and
(C) maintaining said mixture at conditions that amplify a single-stranded nucleic acid molecule (A3) complementary to at least a portion of the second template nucleotide sequence of T3.

66. The method of claim 65 wherein sequence (a) of T3 is exactly identical to at least a portion of the template nucleotide sequence of T2.

67. The method of claim 65 wherein the first, second and third NERSs are identical to each other.

68. A method for amplifying a nucleic acid molecule (A2) comprising:
(a) providing a template nucleic acid molecule (T2) that can hybridize to A2;
(b) providing a primer nucleic acid molecule (A1) that can hybridize to T2 at a location 3′ of the location where A2 can hybridize to T2;
(c) hybridizing A1 to T2;
(d) extending A1 to provide an A1 extension product, where the A1 extension product when hybridized to T2 forms a hybrid H2 that comprises a second nicking agent recognition sequence (NARS) and the nucleotide sequence of A2;
(e) nicking H2 with a second nicking agent (NA) that recognizes the second NARS to thereby form A2;
(f) repeating steps (d) and (e) to thereby amplify A2;
where the primer nucleic acid molecule A1 is formed by a method comprising
(g) providing a template nucleic acid molecule (T1) that can hybridize to A1;
(h) providing a trigger oligonucleotide primer (ODNP) that can hybridize to T1 at a location 3′ of the location where A1 can hybridize to T1;
(i) hybridizing the trigger ODNP to T1;
(j) extending the trigger ODNP to provide a trigger ODNP extension product, where the trigger ODNP extension product when hybridized to T1 forms a hybrid H1 that comprises a first NARS and the nucleotide sequence of A1; and
(k) nicking H1 with a first NA that recognizes the first NARS to thereby form A1.

69. The method of claim 68 wherein the first NARS is identical to the second NARS.

70. The method of claim 68 wherein steps (a)-(j) are performed in a single vessel.

71. The method of claim 68 wherein the first NARS comprises a mismatched base pair.

72. The method of claim 68 wherein both the first and the second NAs are a nicking endonuclease (NE).

73. The method of claim 68 wherein both the first and the second NAs are a restriction endonuclease (RE).

74. A method of amplifying a nucleic acid (A2) comprising
(a) providing a first template nucleic acid (T1) that comprises the sequence of one strand of a first double-stranded nicking agent recognition sequence (NARS) and is at least substantially complementary to a trigger oligonucleotide primer (trigger ODNP);
(b) hybridizing the trigger ODNP to T1;
(c) extending the trigger ODNP to form a hybrid (H1) comprising extended trigger ODNP hybridized to T1, where H1 comprises the first double-stranded NARS;
(d) nicking H1 at a nicking site with a nicking agent (NA) that recognizes the NARS, the fragment having a 5′ end at the nicking site being named A1;
(e) providing a second template nucleic acid (T2) at least substantially complementary to A1;
(f) hybridizing A1 to T2;
(g) extending A1 to form a hybrid (H2) comprising extended A1 hybridized to T2, where H2 comprises a second NARS;
(h) nicking H2 with a second NA that recognizes the second NARS, the fragment having a 5′ terminus at the nicking site being named A2;
(i) extending the 3′ terminus at the nicking site in H2 to re-form H2; and
(j) repeating steps (h) and (i) to thereby amplify A2.

75. The method of claim 74 wherein the first NARS is identical to the second NARS.

76. The method of claim 74 wherein the first NARS comprises a mismatched base pair.

77. The method of claim 74 wherein steps (a)-(j) are performed in a single vessel.

78. The method of claim 74 wherein both the first and the second NAs are a nicking endonuclease (NE).

79. The method of claim 74 wherein both the first and the second NAs are a restriction endonuclease (RE).

80. The method of claim 74 wherein A1 is from 8 to 24 nucleotides in length.

81. The method of claim 74 wherein A2 is from 8 to 24 nucleotides in length.

82. A tandem nucleic acid amplification system comprising:
(a) a first primer extension means for amplifying a first single-stranded nucleic acid (A1); and
(b) a second primer extension means for amplifying a second single-stranded nucleic acid (A2);
where A1 is the primer for the second primer extension means for amplifying A2, and both the first and second primer extension means are contained within a single reaction vessel and require the presence of a nicking agent (NA).

83. The tandem nucleic acid amplification system of claim 82 wherein the NA for the first primer extension means is identical to the NA for the second primer extension means.

84. The tandem nucleic acid amplification system of claim 82, wherein
(a) the first means for amplifying A1 comprises a first oligonucleotide primer (trigger ODNP), a first template nucleic acid (T1) at least substantially complementary to the trigger ODNP, a first nicking agent (NA), a first DNA polymerase, wherein the extension of the trigger ODNP using T1 as a template produces a first nicking agent recognition sequence (NARS) that is recognizable by the first NA; and
(b) the second means for amplifying A2 comprises the nucleic acid (A1), a second template nucleic acid (T2) at least substantially complementary to A1, a second NA, the DNA polymerase, wherein the extension of A1 using T2 as a template produces a second NARS that is recognizable by the second NA.

85. The nucleic acid amplification system of claim 84 wherein the first NA is identical to the second NA.

86. The nucleic acid amplification system of claim 84 or 85 wherein the first polymerase is identical to the second polymerase.

87. The nucleic acid amplification system of claim 84 wherein the first NARS comprises a mismatched base pair.

88. The nucleic acid amplification system of claim 84 wherein both the first NA and the second NAs are a nicking endonuclease (NE).

89. The nucleic acid amplification system of claim 84 wherein both the first and the second NAs are a restriction endonuclease (RE).

90. The nucleic acid amplification system of claim 84 wherein T1 is substantially identical to T2.

91. The nucleic acid amplification system of claim 84 wherein T1 is exactly identical to T2.

92. The nucleic acid amplification system of claim 84 wherein A1 is substantially identical to the trigger ODNP.

93. The nucleic acid amplification system of claim 84 wherein A1 is exactly identical to the trigger ODNP.

94. The nucleic acid amplification system of claim 84 wherein A2 is substantially identical to the trigger ODNP.

95. The nucleic acid amplification system of claim 84 wherein A2 is exactly identical to the trigger ODNP.

96. The nucleic acid amplification system of claim 95 wherein A1 is not substantially identical to A2.

97. The nucleic acid amplification system of claim 84 wherein A1 is substantially identical to the trigger ODNP.

98. The nucleic acid amplification system of claim 84 wherein A1 is exactly identical to the trigger ODNP.

99. The nucleic acid amplification system of claim 84 wherein A1 is from 8 to 24 nucleotides in length.

100. The nucleic acid amplification system of claim 84 wherein A2 is from 8 to 24 nucleotides in length.

101. A method for exponential amplification of a nucleic acid molecule A2 comprising
(a) amplifying a nucleic acid molecule (A1) using a first template nucleic acid (T1) that comprises the sequence of one strand of a first nicking agent recognition sequence (NARS) as a template in the presence of a first nicking endonuclease (NA) that recognizes the first NARS and a first DNA polymerase; and
(b) amplifying A2 using a second template nucleic acid (T2) that comprises the sequence of one strand of a second NARS as a template and A1 as an primer in the presence of a second NA and a second DNA polymerase.

102. The method of claim 101 wherein the first NARS is identical to the second NARS.

103. The method of claim 101 wherein the first NARS comprises a mismatched base pair.

104. The method of claim 101 or claim 102 wherein the first DNA polymerase is identical to the second DNA polymerase.

105. The method of claim 101 wherein steps (a) and (b) are performed in a single vessel.

106. The method of claim 101 wherein the NA is a nicking endonuclease (NE).

107. The method of claim 106 wherein the NA is a restriction endonuclease (RE).

108. A method for amplifying a nucleic acid molecule, comprising:
(A) forming a mixture comprising
(i) a first single-stranded nucleic acid molecule having a sequence (S1);
(ii) a second single-stranded nucleic acid molecule having a sequence of an antisense strand of a nicking agent recognition sequence (NARS), wherein a sequence substantially complementary to S1 is present both 3′ and 5′ to the sequence of the antisense strand of the NARS;
(iii) a nicking agent (NA) that recognizes the NARS; a DNA polymerase; and one or more deoxynucleoside triphosphate(s); and
(B) maintaining said mixture at conditions that amplify a single-stranded nucleic acid molecule using single-stranded nucleic acid molecule (A)(ii) as a template.

109. The method of claim 108 wherein the sequence in the single-stranded nucleic acid molecule (A) (ii) that is at least substantially complementary to S1 is exactly complementary to S1.

110. The method of claim 108 wherein the amplified nucleic acid molecule has a sequence that is exactly identical to S1.

111. The method of any one of claims 3, 27, 53, 72, 78, 88, and 106 wherein the NE is N.BstNB I or N.AIw I.

112. The method of claim 111 wherein the both the first and the second NEs are N.BstNB I.

113. The method of any one of claims 1, 24, 53, 68, 74 and 108 wherein the amplification is performed under isothermal conditions.

114. The method of claim 113 wherein each amplification reaction is performed at 50° C.-70° C.

115. The method of claim 113 wherein each amplification reaction is performed at 60° C.

116. The method of claims 1-101 wherein each amplification reaction is performed at temperatures between a highest temperature and a lowest temperature, where the highest temperature is within 20° C. of the lowest temperature.

117. The method of claim 116 wherein the highest temperature is within 15° C. of the lowest temperature.

118. The method of claim 116 wherein the highest temperature is within 10° C. of the lowest temperature.

119. The method of claim 116 wherein the highest temperature is within 5° C. of the lowest temperature.

120. The method of any one of claims 1-101 wherein the DNA polymerase is 5′→3′ exonuclease deficient.

121. The method of claim 120 wherein the 5′→3′ exonuclease deficient DNA polymerase is selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase and T4 DNA polymerase homoenzyme.

122. The method of claim 120 wherein the 5′→3′ exonuclease deficient DNA polymerase is exo⁻ Bst polymerase, exo⁻ Bca polymerase, exo⁻ Vent polymerase, 9° Nm™ DNA polymerase or exo⁻ Deep Vent polymerase.

123. The method of any one of claims 1, 24, 53, 68, 74,101, and 108 wherein the DNA polymerase has a strand displacement activity.

124. The method of any one of claims 1-101 wherein each amplification reaction is performed in the presence of a strand displacement facilitator.

125. The method of claim 124 wherein the strand displacement facilitator is selected from the group consisting of BMRF1 polymerase accessory subunit, adenovirus DNA-binding protein, herpes simplex viral protein ICP8, single-stranded DNA binding proteins, phage T4 gene 32 protein, calf thymus helicase, and trehalose.

126. The method of claim 125 wherein the strand displacement facilitator is trehalose.

127. A composition comprising:
(a) a first at least partially double-stranded nucleic acid molecule of which one strand comprises a sequence of an antisense strand of a first nicking agent recognition sequence (NARS); and
(b) a second at least partially double-stranded nucleic acid molecule of which one strand comprises, from 5′ to 3′:
(i) a sequence of an antisense strand of a second NARS, and
(ii) a sequence that is at least substantially identical to a sequence located 5′ to the sequence of the antisense strand of the first NARS in the first nucleic acid.

128. The composition of claim 127 wherein the first NARS is recognizably by a first nicking endonuclease, and the second NARS is recognizable by a second nicking endonuclease.

129. The composition of claim 127 wherein the first NARS is recognizably by a first restriction endonuclease, and the second NARS is recognizable by a second restriction endonuclease.

130. The composition of claim 127 wherein the first NARS is identical to the second NARS.

131. The composition of claim 130 wherein both the first and the second NARSs are recognizable by a nicking endonuclease (NE).

132. The composition of claim 130 wherein both the first and the second NARSs are recognizable by a restriction endonuclease (RE).

133. The composition of claim 127 wherein sequence (b) (ii) is exactly identical to a sequence located 5′ to the sequence of the antisense strand of the first NARS in the first nucleic acid.

134. A composition comprising:
(a) a first at least partially double-stranded nucleic acid molecule of which one strand comprises a sequence of a sense strand of a first nicking agent recognition sequence (NARS); and
(b) a second at least partially double-stranded nucleic acid molecule of which one strand comprises from 5′ to 3′:
(i) a sequence of an antisense strand of a second NARS, and
(ii) a sequence that is at least substantially complementary to a sequence located 3′ to the sequence of the sense strand of the first NARS in the first nucleic acid.

135. The composition of claim 134 wherein the first NARS is recognizably by a first nicking endonuclease, and the second NARS is recognizable by a second nicking endonuclease.

136. The composition of claim 134 wherein the first NARS is recognizably by a first restriction endonuclease, and the second NARS is recognizable by a second restriction endonuclease.

137. The composition of claim 134 wherein the first NARS is identical to the second NARS.

138. The composition of claim 137 wherein the first and second NARSs are recognizable by a nicking endonuclease.

139. The composition of claim 137 wherein the first and second NARSs are recognizable by a restriction endonuclease.

140. The composition of claim 134 wherein sequence (b) (ii) is exactly complementary to a sequence located 3′ to the sequence of the sense strand of the NARS in the first nucleic acid.

141. The composition of claim 127 or claim 134 further comprising a first NA that recognizes the first NARS and a second NA that recognizes the second NARS.

142. The composition of claim 130 or claim 137 further comprising a nicking agent that recognizes both the first and second NARSs.

143. The composition of claim 131 or claim 138 further comprising a nicking endonuclease (NE) that recognizes both the first and the second NERSs.

144. The composition of claim 132 or claim 139 further comprising a restriction endonuclease (RE) that recognizes both the first and the second NARSs.

145. The composition of claim 143 wherein the NE is N.BstNB I.

146. The composition of claim 127 or claim 134 further comprising a DNA polymerase.

147. The composition of claim 146 wherein the DNA polymerase is 5′→3′ exonuclease deficient.

148. The composition of claim 146 wherein the DNA polymerase is selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase and T4 DNA polymerase homoenzyme.

149. The composition of claim 146 wherein the 5′→3′ exonuclease deficient DNA polymerase is exo⁻ Bst polymerase, exo⁻ Bca polymerase, exo⁻ Vent polymerase, 9° Nm™ DNA polymerase, or exo⁻ Deep Vent polymerase.

150. The composition of claim 146 wherein the DNA polymerase has a strand displacement activity.

151. The composition of claim 127 or claim 134 further comprising a strand displacement facilitator.

152. The composition of claim 151 wherein the strand displacement facilitator is selected from the group consisting of BMRF1 polymerase accessory subunit, adenovirus DNA-binding protein, herpes simplex viral protein ICP8, single-stranded DNA binding proteins, phage T4 gene 32 protein, calf thymus helicase, and trehalose.

153. The composition of claim 152 wherein the strand displacement facilitator is trehalose.

154. The composition of claim 143 further comprising a DNA polymerase and a strand displacement facilitator.

155. The composition of claim 144 further comprising a DNA polymerase and a strand displacement facilitator.

156. The composition of claim 127 or claim 154, further comprising a labeled deoxynucleoside triphosphate, a labeled oligonucleotide that is at least substantially complementary to a sequence located 5′ to the sequence of the antisense strand of the second NARS in T2, or a fluorescent intercalating agent.

157. The composition of claim 134 or claim 155, further comprising a labeled deoxynucleoside triphosphate, a labeled oligonucleotide that is at least substantially complementary to a sequence located 5′ to the sequence of the antisense strand of the second NERS in T2, or a fluorescent intercalating agent.

158. An isolated single-stranded nucleic acid molecule, from 3′ to 5′, consisting essentially of:
(i) a sequence that is 6-100 nucleotides in length;
(ii) a sequence of the antisense strand of a nicking agent recognition sequence (NARS); and
(iii) a sequence that is at most 100 nucleotides in length.

159. The isolated single-stranded nucleic acid molecule of claim 158 wherein the NARS is recognizable by a nicking endonuclease (NE).

160. The isolated single-stranded nucleic acid molecule of claim 158 wherein the NARS is recognizable by a restriction endonuclease (RE).

161. The isolated single-stranded nucleic acid molecule of claim 158 wherein sequence (i) is from 8 to 24 nucleotides in length.

162. The isolated single-stranded nucleic acid molecule of claim 158 wherein sequence (i) is from 12 to 17 nucleotides in length.

163. The isolated single-stranded nucleic acid molecule of claim 158 wherein the isolated nucleic acid molecule is at most 200 nucleotides in length.

164. The isolated single-stranded nucleic acid molecule of claim 158 wherein the isolated nucleic acid molecule is at most 100 nucleotides in length.

165. The isolated single-stranded nucleic acid molecule of claim 158 wherein the isolated nucleic acid molecule is at most 50 nucleotides in length.

166. The isolated single-stranded nucleic acid molecule of claim 158 wherein the isolated nucleic acid molecule is at most 30 nucleotides in length.

167. The isolated single-stranded nucleic acid molecule of claim 158 wherein a portion of sequence (iii) at the 5′ terminus of the isolated nucleic acid molecule is at least substantially identical to a portion of sequence (i) that is at least 6 nucleotides in length.

168. The isolated single-stranded nucleic acid molecule of claim 158 wherein the portion of sequence (iii) at the 5′ terminus of the isolated nucleic acid molecule is exactly identical to the portion of sequence (i) that is at least 6 nucleotides in length.

169. The isolated single-stranded nucleic acid molecule of claim 158 and claim 167 wherein the isolated single-stranded nucleic acid molecule is immobilized to a substrate.

170. The isolated single-stranded nucleic acid molecule of claim 169 wherein the isolated single-stranded nucleic acid is covalently immobilized to the substrate.

171. The isolated single-stranded nucleic acid molecule of claim 169 wherein the isolated single-stranded nucleic acid is non-covalently immobilized to the substrate.

172. The isolated single-stranded nucleic acid molecule of claim 169 wherein the substrate comprises a material selected from the group consisting of silicon, glass, paper, ceramic, metal, metalloid and plastics.

173. The isolated single-stranded nucleic acid molecule of claim 169 wherein the isolated single-stranded nucleic acid is immobilized to the substrate via a linker.

174. A composition comprising the isolated single-stranded nucleic acid molecule of claim 158 and an oligonucleotide primer (trigger ODNP) that is at least substantially complementary to sequence (i).

175. The composition of claim 174 wherein the trigger ODNP is exactly complementary to sequence (i).

176. The composition of claim 174 further comprising a nicking agent (NA) that recognizes the NARS.

177. The composition of claim 176 wherein the NA is a nicking endonuclease (NE).

178. The composition of claim 177 wherein the NE is N.BstNB I or N.AIw 1.

179. The composition of claim 178 wherein the NE is N.BstNB I.

180. The composition of claim 174 or 177 further comprising a DNA polymerase.

181. The composition of claim 180 wherein the DNA polymerase is 5′→3′ exonuclease deficient.

182. The composition of claim 181 wherein the 5′→3′ exonuclease deficient DNA polymerase is selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ polymerase, and T4 DNA polymerase homoenzyme.

183. The composition of claim 182 wherein the 5′→3′ exonuclease deficient DNA polymerase is exo⁻ Bst polymerase, exo⁻ Bca polymerase, exo⁻ Vent polymerase, 9° Nm™ polymerase, or exo⁻ Deep Vent polymerase.

184. The composition of claim 180 wherein the DNA polymerase has a strand displacement activity.

185. The composition of any one of claims 174,176 and 180 further comprising a strand displacement facilitator.

186. The composition of claim 185 wherein the strand displacement facilitator is selected from the group consisting of BMRF1 polymerase accessory subunit, adenovirus DNA-binding protein, herpes simplex viral protein ICP8, single-stranded DNA binding proteins, phage T4 gene 32 protein, calf thymus helicase, and trehalose.

187. The composition of claim 185 wherein the strand displacement facilitator is trehalose.

188. An array, comprising:
(a) a substrate having a plurality of distinct areas; and
(b) a plurality of single-stranded nucleic acids immobilized to the distinct areas wherein a single-stranded nucleic acid in the plurality is the isolated single-stranded nucleic acid of claim 158 or claim 1580.

189. The array of claim 188 wherein the single-stranded nucleic acid molecules in any one of the distinct areas are homogeneous, but different from the single-stranded nucleic acid molecules in another distinct area.

190. The array of claim 188 wherein the single-stranded nucleic acid molecules in at least one of the distinct areas are heterogeneous.

191. The array of claim 188 wherein the plurality of single-stranded nucleic acids are covalently immobilized to the substrate.

192. The array of claim 188 wherein the plurality of single-stranded nucleic acids are non-covalently immobilized to the substrate.

193. The array of claim 188 wherein the substrate is made of a material selected from the group consisting of silicon, glass, paper, ceramic, metal, metalloid, and plastic.

194. A composition comprising:
(a) a first at least partially double-stranded nucleic acid molecule of which one strand comprises, from 3′ to 5′:
(i) a first sequence (S1′) at least 8 nucleotides in length,
(ii) a sequence of an antisense strand of a first NARS, and
(iii) a second sequence (S2′) that is at least 8 nucleotides in length and is not substantially identical to S1′; and
(b) a second at least partially double-stranded nucleic acid molecule of which one strand comprises, from 3′ to 5′:
(i) a sequence that is at least substantially identical to S2′,
(ii) a sequence of an antisense strand of a second NARS, and
(iii) a sequence that is at least substantially identical to S1′.

195. The composition of claim 194 wherein the first NARS is identical to the second NARS.

196. The composition of claim 194 wherein both the first and the second NARSs are recognizable by a nicking endonuclease (NE).

197. The composition of claim 194 wherein both the first and the second NARS is recognizable by a restriction endonuclease (RE).

198. The composition of claim 194 wherein sequence (b)(i) is exactly identical to S2′.

199. The composition of claim 194 wherein sequence (b)(iii) is exactly identical to S1′.

200. An isolated single-stranded nucleic acid molecule, comprising at least two sequences of an antisense strand of a nicking agent recognition sequence (NARS).

201. The isolated single-stranded nucleic acid molecule of claim 200 wherein the nucleic acid molecule is at most 100 nucleotides in length.

202. The isolated single-stranded nucleic acid molecule of claim 200 wherein the shortest distance between two of the at least two sequences is no more than 50 nucleotides.

203. The isolated single-stranded nucleic acid molecule of claim 200 wherein the shortest distance between two of the at least two sequences is no more than 25 nucleotides.

204. A method for determining the presence or the absence of a target nucleic acid in a sample, comprising
(A) forming a mixture comprising:
(i) the nucleic acid molecules of the sample;
(ii) a first single-stranded nucleic acid molecule (T1) comprising from 3′ to 5′:
(a) a first sequence that is at least substantially complementary to the target nucleic acid,
(b) a sequence of the antisense strand of a first nicking agent recognition sequence (NARS), and
(c) a second sequence;
(iii) a second single-stranded nucleic acid molecule (T2) comprising from 3′ to 5′:
(a) a first sequence that is at least substantially identical to the second sequence of T1,
(b) a sequence of the antisense strand of a second NARS, and
(c) a second sequence; and
(iv) a first nicking endonuclease (NA) that recognizes the first NARS, a second NA that recognizes the second NARS, a DNA polymerase, and one or more deoxynucleoside triphosphate(s);
(B) maintaining the mixture at conditions that amplify a single-stranded nucleic acid molecule (A2) using the second sequence of T2 as a template if the target nucleic acid is present in the sample; and
(C) detecting the presence or the absence of A2 to determine the presence, or the absence, of the target nucleic acid in the sample.

205. The method of claim 204 wherein the first NARS and the second NARS are identical and recognizable by a nicking endonuclease.

206. A method for determining the presence or the absence of a target nucleic acid in a sample, comprising
(A) form a mixture comprising:
(i) the nucleic acid molecules of the sample;
(ii) a first single-stranded nucleic acid molecule (T1) comprising from 3′ to 5′:
(a) a sequence that is at least substantially complementary to the target nucleic acid, and
(b) a sequence of the sense strand of a first nicking agent recognition sequence (NARS),
(iii) a second single-stranded nucleic acid molecule (T2) comprising from 3′ to 5′:
(a) a sequence that is at least substantially complementary to the sequence of T1 that is located 3′ to the sequence of the sense strand of the first NERS, and
(b) a sequence of the antisense strand of a second NARS; and
(iv) a first nicking endonuclease (NA) that recognizes the first NARS, a second NA that recognizes the second NARS, a DNA polymerase, and one or more deoxynucleoside triphosphate(s);
(B) maintaining the mixture at conditions that amplify a single-stranded nucleic acid molecule (A2) using T2 as a template if the target nucleic acid is present in the sample; and
(C) detecting the presence or the absence of A2 to determine the presence, or the absence, of the target nucleic acid in the sample.

207. The method of claim 206 wherein the first and second NARS are identical.

208. A method for determining the presence or absence of a target nucleic acid that comprises a first nicking endonuclease recognition sequence (NERS) in a sample, the method comprising:
(A) forming a mixture comprising:
(i) the nucleic acid molecules of the sample,
(ii) a single-stranded nucleic acid molecule (T2) comprising from 3′ to 5′:
(a) a sequence that is at least substantially identical to a portion of the target nucleic acid molecule located 5′ to the sequence of the antisense strand of the first NERS, and
(b) a sequence of the antisense strand of a second NERS, and
(iii) a first nicking endonuclease (NE) that recognizes the first NERS; a second NE that recognizes the second NERS, a DNA polymerase, and one or more deoxynucleoside triphosphate(s);
(B) maintaining the mixture at conditions that amplify a single-stranded nucleic acid molecule (A2) using T2 as a template if the target nucleic acid is present in the sample; and
(C) detecting the presence or absence of A2 to determine the presence or absence of the target nucleic acid in the sample.

209. The method of claim 208 wherein the first NERS is identical to the second NERS.

210. A method for determining the presence or absence of a target nucleic acid that comprises a first nicking endonuclease recognition sequence (NERS) in a sample, the method comprising:
(A) forming a mixture comprising:
(i) the nucleic acid molecules of the sample,
(ii) a first single-stranded nucleic acid molecule (T1) that is substantially identical to one strand of the target nucleic acid and comprise a sequence of the antisense strand of the first NERS,
(iii) a second single-stranded nucleic acid molecule (T2) comprising from 3′ to 5′:
(a) a sequence that is at least substantially identical to a portion of T1 located 5′ to the sequence of the antisense strand of the first NERS, and
(b) a sequence of the antisense strand of a second NERS, and
(iv) a first nicking endonuclease (NE) that recognizes the first NERS; a second NE that recognizes the second NERS, a DNA polymerase, and one or more deoxynucleoside triphosphate(s);
(B) maintaining the mixture at conditions that amplify a single-stranded nucleic acid molecule (A2) using T2 as a template if the target nucleic acid is present in the sample; and
(C) detecting the presence or absence of A2 to determine the presence or absence of the target nucleic acid in the sample.

211. The method of claim 210 wherein the first NERS is identical to the second NERS.

212. The method of claim 210 wherein A2 has at most 25 nucleotides.

213. A method for determining the presence or absence of a target nucleic acid in a sample, comprising
(A) forming a mixture of a first oligonucleotide primer (ODNP), a second ODNP, and the nucleic acid molecules of the sample, wherein
(i) if the target nucleic acid is a double-stranded nucleic acid having a first strand and a second strand,
the first ODNP comprises a nucleotide sequence of a sense strand of a first restriction endonuclease recognition sequence (RERS) and a nucleotide sequence that is at least substantially complementary to a first portion of the first strand of the target nucleic acid, and
the second ODNP comprises a nucleotide sequence that is at least substantially complementary to a second portion of the second strand of the target nucleic acid and comprises a sequence of the sense strand of a second RERS, the second portion being located 3′ to the complement of the first portion in the second strand of the target nucleic acid, or
(ii) if the target nucleic acid is a single-stranded nucleic acid,
the first ODNP comprises a nucleotide sequence of a sense strand of a first RERS and a nucleotide sequence that is at least substantially identical to a first portion of the target nucleic acid, and
the second ODNP comprises a nucleotide sequence that is at least substantially complementary to a second portion of the target nucleic acid and comprises a sequence of the sense strand of a second RERS, the second portion being located 5′ to the first portion in the target nucleic acid;
(B) subjecting the mixture to conditions that, if the target nucleic acid is present in the sample,
(i) extend the first and the second ODNPs to produce an extension product comprising both the first and the second RERSs;
(ii) amplify a first single-stranded nucleic acid fragment (A1) using one strand of the extension product of step (B)(i) as a template in the presence of one or more restriction endonucleases (REs) that recognizes the first and the second RERSs;
(iii) in the presence of a second single-stranded nucleic acid molecule (T2) capable of annealing to A1, amplify a third single-stranded nucleic acid fragment (A2) using A1 as a template, wherein A1, A2 or both have at most 25 nucleotides, and wherein T2 comprising, from 5′ to 3′:
(a) a sequence of the antisense strand of a third RERS, and
(b) a sequence that is at least substantially complementary to A1; and
(C) detecting the presence or absence of A2 to determine the presence or absence of the target nucleic acid in the sample.

214. The method of claim 213 wherein the first RERS is identical to the second RERS.

215. A method for determining the presence or absence of a target nucleic acid in a sample, comprising
(A) forming a mixture of a first oligonucleotide primer (ODNP), a second ODNP, and the nucleic acid molecule of the sample, wherein
(i) if the target nucleic acid is a double-stranded nucleic acid having a first strand and a second strand,
the first ODNP comprises a nucleotide sequence of a sense strand of a first nicking endonuclease recognition sequence (NERS) and a nucleotide sequence that is at least substantially complementary to a first portion of the first strand of the target nucleic acid, and
the second ODNP comprises a nucleotide sequence that is at least substantially complementary to a second portion of the second strand of the target nucleic acid and comprises a sequence of the sense strand of a second NERS, the second portion being located 3′ to the complement of the first portion in the second strand of the target nucleic acid, or
(ii) if the target nucleic acid is a single-stranded nucleic acid,
the first ODNP comprises a nucleotide sequence of a sense strand of a first NERS and a nucleotide sequence that is at least substantially identical to a first portion of the target nucleic acid, and
the second ODNP comprises a nucleotide sequence that is at least substantially complementary to a second portion of the target nucleic acid and comprises a sequence of the sense strand of a second NERS, the second portion being located 5′ to the first portion in the target nucleic acid;
(B) subjecting the mixture to conditions that, if the target nucleic acid is present in the sample,
(i) extend the first and the second ODNPs to produce an extension product comprising both the first and the second NERSs;
(ii) amplify a first single-stranded nucleic acid fragment (A1) using one strand of the extension product of step (B)(i) as a template in the presence of one or more nicking endonucleases (NEs) that recognizes the first and the second NERSs;
(iii) in the presence of a second single-stranded nucleic acid molecule (T2) capable of annealing to A1, amplify a third single-stranded nucleic acid fragment (A2) using A1 as a template, wherein A1, A2 or both have at most 25 nucleotides, and wherein T2 comprising, from 5′ to 3′:
(a) a sequence of the antisense strand of a third NERS, and
(b) a sequence that is at least substantially complementary to A1; and
(C) detecting the presence or absence of A2 to determine the presence or absence of the target nucleic acid in the sample.

216. The method of claim 215 wherein the first, second and third NERSs are identical.

217. A method for determining the presence or absence of a target nucleic acid in a sample, comprising
(A) forming a mixture comprising:
(i) the nucleic acid molecules of the sample,
(ii) a single-stranded nucleic acid probe that comprises, from 3′ to 5′, a sequence that is at least substantially complementary to the 5′ portion of the target nucleic acid, and a sequence of the antisense strand of a first nicking agent recognition sequence (NARS),
(B) removing unhybridized probe from the mixture of step (A);
(C) performing an amplification reaction in the presence of a first nicking agent (NA) that recognizes the first NARS;
(D) providing a single-stranded nucleic acid molecule (T2) comprising, from 5′ to 3′:
(i) a sequence of the antisense strand of a second NARS, and
(ii) a sequence that is at least substantially identical to the portion of the first single-stranded nucleic acid probe located 5′ to the sequence of the antisense strand of the first NARS,
(E) performing an amplification reaction in the presence of a second NA that recognizes the second NARS;
(F) detecting the presence or absence of the amplification product of step (E) to determine the presence or absence of the target nucleic acid in the sample.

218. The method of claim 217 wherein the first and second NARSs are identical.

219. A method for determining the presence or absence of a target nucleic acid in a sample, comprising
(A) forming a mixture comprising:
(i) the nucleic acid molecules of the sample,
(ii) a single-stranded nucleic acid probe that comprises, from 5′ to 3′, a sequence that is at least substantially complementary to the 3′ portion of the target nucleic acid, and a sequence of the antisense strand of a first NARS;
(B) removing unhybridized probe from the mixture of step (A);
(C) performing an amplification reaction in the presence of a first nicking agent (NA) that recognizes the first NARS;
(D) providing a single-stranded nucleic acid molecule (T2) comprising, from 5′ to 3′:
(i) a sequence of the antisense strand of a second NARS, and
(ii) a sequence that is at least substantially complementary to the portion of the first single-stranded nucleic acid probe located 5′ to the sequence of the antisense strand of the first NARS,
(E) performing an amplification reaction in the presence of a second NA that recognizes the second NARS; and
(F) detecting the presence or absence of the amplification product of step (E) to determine the presence or absence of the target nucleic acid in the sample.

220. The method of claim 219 wherein the first and second NARS are identical.

221. A method for determining the presence or absence of a target nucleic acid in a sample, comprising
(A) forming a mixture comprising:
(i) the nucleic acid molecules of the sample,
(ii) a partially double-stranded nucleic acid probe that comprises:
(a) a sequence of a sense strand of a first NARS, a sequence of an antisense strand of the first NARS, or both; and
(b) a 5′ overhang in the strand that the strand itself or an extension product thereof contains a nicking site (NS) nickable by a first nicking agent (NA) that recognizes the first NARS, or
a 3′ overhang in the strand that neither the strand nor an extension product thereof contains the NS,
wherein each overhang comprises a nucleic acid sequence that is at least substantially complementary to the target nucleic acid;
(B) removing unhybridized probe from the mixture of step (A);
(C) performing an amplification reaction in the presence of a first nicking agent (NA) that recognizes the first NARS;
(D) providing a single-stranded nucleic acid molecule (T2) comprising, from 5′ to 3′:
(i) a sequence of the antisense strand of a second NARS, and
(ii) a sequence that is at least substantially identical to the portion of the nucleic acid probe located 5′ to the sequence of the antisense strand of the first NARS,
(E) performing an amplification reaction in the presence of a second NA that recognizes the second NARS;
(F) detecting the presence or absence of the amplification product of step (E) to determine the presence or absence of the target nucleic acid in the sample.

222. The method of claim 221 wherein the first and second NARSs are identical.

223. A method for determining the presence or absence of a genetic variation at a defined location in a single-stranded target nucleic acid, comprising:
(A) providing a single-stranded nucleic acid (A1) that comprises a sequence that is exactly complementary to a portion of the target nucleic acid, the portion of the target nucleic acid comprising a nucleotide or nucleotides at the defined location, the A1 being amplified in the presence of a first nicking agent;
(B) performing an amplification reaction in the presence of
(i) a single-stranded template nucleic acid (T2) that comprises, from 3′ to 5′:
(a) a first sequence that is at least substantially complementary to the A1 and comprises the genetic variation,
(b) a sequence of the antisense strand of a nicking agent recognition sequence that is recognizable by a second nicking agent,
(c) a second sequence,
(ii) the second nicking agent,
(iii) a DNA polymerase, and
(iv) one or more deoxynucleoside triphosphates,
under conditions that amplify a single-stranded nucleic acid molecule (A2) using at least a portion of the second sequence of the T2 molecule only if the A1 comprises the complementary nucleotide(s) of the genetic variation, and
(C) detecting the presence or absence of the A2 molecule to determine the presence or absence of the genetic variation at the defined location of the target nucleic acid.

224. The method of claim 223 wherein the first nicking agent is identical to the second nicking agent.

225. The method of claim 223 wherein the single-stranded target nucleic acid is one strand of a denatured double-stranded nucleic acid.

226. The method of claim 223 wherein the A1 is at most 25 nucleotides in length.

227. The method of claim 223 wherein the A1 is at most 17 nucleotides in length.

228. The method of claim 223 wherein the A1 is at most 12 nucleotides in length.

229. The method of claim 223 wherein the A1 is provided by
(a) forming a mixture of a first ODNP, a second ODNP, and the target nucleic acid, wherein
(i) the first ODNP comprises a nucleotide sequence of one strand of a first RERS and a nucleotide sequence that is at least substantially identical to a nucleotide sequence of the target nucleic acid located 5′ to the complement of the genetic variation, and
(ii) the second ODNP comprises a sequence of one strand of a second RERS and a nucleotide sequence that is at least substantially complementary to a nucleotide sequence of the target nucleic acid located 3′ to the genetic variation;
(b) extending the first and the second ODNPs in the presence of deoxyribonucleoside triphosphates and at least one modified deoxyribonucleoside triphosphate to produce an extension product comprising both the first and the second RERSs; and
(c) amplifying the single-stranded nucleic acid fragment A1 using one strand of the extension product of step (b) as a template in the presence of restriction endonucleases (REs) that recognize the first RERS and the second RERS.

230. The method of claim 229 wherein the first, second and third RERSs are identical to each other.

231. The method of claim 223 wherein the A1 is provided by
(a) forming a mixture of a first oligonucleotide primer (ODNP), a second ODNP and the target nucleic acid, wherein
(i) the first ODNP comprises a nucleotide sequence that is at least substantially identical to a nucleotide sequence of the target nucleic acid located 5′ to the genetic variation, and
(ii) the second ODNP comprises a nucleotide sequence that is at least substantially complementary to a nucleotide sequence of the target nucleic acid located 3′ to the genetic variation,
the first and the second ODNPs each further comprising a nucleotide sequence of a sense strand of a nicking endonuclease recognition sequence (NERS);
(b) extending the first and the second ODNPs to produce an extension product comprising two NERSs; and
(c) amplifying the single-stranded nucleic acid fragment A1 using one strand of the extension product of step (b) as a template in the presence of one or more nicking endonucleases (NEs) that recognizes the NERS(s).

232. The method of claim 231 wherein the NERSs in the first ODNP, the second ODNP and T2 are identical to each other.

233. The method of claim 223 wherein the genetic variation is a single nucleotide polymorphism.

234. The method of claim 223 wherein the genetic variation is associated with a disease.

235. The method of claim 223 wherein the disease is a human genetic disease.

236. The method of claim 223 wherein the genetic variation is associated with drug resistance of a pathogenic microorganism.

237. The method of claim 224 wherein the nicking agent is N.BstNB I.

238. The method of claim 223 wherein step (B) is performed under an isothermal condition.

239. The method of claim 238 wherein step (B) is performed at 50° C.-70° C.

240. The method of claim 223 wherein the DNA polymerase is selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ DNA polymerase, and T4 DNA polymerase homoenzyme.

241. The method of claim 240 wherein the DNA polymerase is exo⁻ Vent, exo⁻ Deep Vent, exo⁻ Bst, exo⁻ Bca, or 9° Nm™ DNA polymerase.

242. The method of claim 223 wherein step (C) is performed at least partially by the use of a technique selected from the group consisting of mass spectrometry, liquid chromatography, fluorescence polarization, and electrophoresis.

243. The method of claim 223 wherein step (C) is performed at least partially by the use of liquid chromatography.

244. The method of claim 223 wherein step (C) is performed at least partially by the use of mass spectrometry.

245. The method of claim 223 wherein step (C) is performed at least partially by both liquid chromatography and mass spectrometry.

246. The method of claim 229 or claim 231 wherein the first ODNP, the second ODNP or both are immobilized.

247. The method of claim 223 wherein the target nucleic acid is immobilized.

248. The method of claim 223 wherein the T2 is immobilized to a solid support.

249. The method of claim 223 or claim 248 wherein the second sequence of the T2 is at least substantially identical to the first sequence and comprises the genetic variation.

250. The method of claim 249 wherein the second nicking agent nicks 5′ to the sequence of the sense strand of the nicking agent recognition sequence, and wherein the portion of the second sequence of the T2 located immediately 5′ to the nicking site nickable by the second nicking agent is exactly identical to the first sequence of the T2 molecule.

251. A method for identifying a genetic variation at a defined location in a single-stranded target nucleic acid, comprising:
(A) providing a single-stranded nucleic acid (A1) that comprises a sequence that is exactly complementary to a portion of the target nucleic acid, the portion of the target nucleic acid comprising genetic variation at the defined location, the A1 being amplified in the presence of a first nicking agent;
(B) performing an amplification reaction in the presence of
(i) multiple single-stranded template nucleic acids (T2), each of the multiple single-stranded template nucleic acids comprises, from 3′ to 5′:
(a) a first sequence that is at least substantially complementary to the A1 and comprises one of the potential genetic variations at the defined position of the target nucleic acid,
(b) a sequence of the antisense strand of a nicking agent recognition sequence that is recognizable by a second nicking agent,
(c) a second sequence that uniquely correlates to the potential genetic variation,
wherein the multiple T2 molecules, in combination, comprise all the potential genetic variations at the defined position of the target nucleic acid,
(ii) the second nicking agent,
(iii) a DNA polymerase, and
(iv) one or more deoxynucleoside triphosphates,
under conditions that selectively amplify a single-stranded nucleic acid molecule (A2) using at least a portion of the second sequence of a T2 molecule as a template, the T2 molecule comprising the genetic variation of the target nucleic acid, and
(C) characterizing the A2 amplified in step (B) to identify the gene variation of the target nucleic acid.

252. The method of claim 251 wherein the second sequence of each of the T2 molecules is at least substantially identical to the first sequence of the same T2 molecule.

253. The method of claim 252 wherein the second nicking agent nicks 5′ to the sequence of the sense strand of the nicking agent recognition sequence, and wherein the portion of the second sequence of each of T2 molecule located immediately 5′ to the nicking site nickable by the second nicking agent is exactly identical to the first sequence of the same T2 molecule.

254. The method of claim 158, claim 159 or claim 160 wherein each of the T2 molecules is immobilized to a solid support.

255. The method of claim 251 wherein the single-stranded target nucleic acid is one strand of a denatured double-stranded nucleic acid.

256. A method for determining the presence or absence of a junction between an upstream exon (Exon A) and a downstream exon (Exon B) in a cDNA molecule, comprising:
(A) providing an at least partially double-stranded nucleic acid molecule (N1) comprising
(i) at least one of a sequence of the sense strand of a first nicking agent recognition sequence (NARS) and a sequence of the antisense strand of the first NARS, and
(ii) at least one strand of a portion of the cDNA molecule if the cDNA molecule is double-stranded, or a portion of the cDNA is the cDNA molecule is single-stranded, the portion being suspected to contain the junction between Exon A and Exon B;
(B) amplifying a first single-stranded nucleic acid molecule (A1) in the presence of a nicking agent (NA) that recognizes the first NARS, a DNA polymerase, and one or more deoxynucleoside triphosphate(s), wherein the amplifying uses the portion of the cDNA as a template for the polymerase;
(C) providing a second single-stranded nucleic acid molecule (T2) comprising, from 5′ to 3′:
(i) a first sequence comprising
(a) a 3′ portion of the sense strand of Exon A linked at the 3′ terminus of the 3′ portion to a 5′ portion of the sense strand of Exon B at the 5′ terminus of the 5′ terminus, or
(b) a 5′ portion of the antisense strand of Exon A linked at the 5′ terminus of the 5′ portion to a 3′ portion of the antisense strand of Exon B at the 3′ terminus of the 3′ portion,
wherein if the cDNA contains the junction between Exon A and Exon B, the first sequence of the T2 is at least substantially complementary to the A1 molecule, but if the cDNA does not contain the junction between Exon A and Exon B, the T2 is not substantially complementary to the A1 molecule,
(ii) a sequence of the antisense strand of a second NARS, and
(iii) a second sequence;
(D) performing an amplification reaction that amplify a third single-stranded nucleic acid molecule (A2) using at least a portion of the second sequence of T2 as a template if the junction between Exon A and Exon B is present in the target cDNA molecule; and
(E) detecting the presence or absence of the A2 to determine the presence or absence of the junction in the cDNA molecule.

257. The method of claim 256 wherein the first NARS is identical to the second NARS.

258. The method of claim 256 wherein both the first and the second NAs are nicking endonucleases (NEs).

259. The method of claim 258 wherein both the first and the second NAs are N.BstNB I.

260. The method of claim 257 wherein both the first and second NAS are a nicking endonuclease (NE).

261. The method of claim 256 wherein steps (A)-(D) are performed in a single vessel.

262. The method of claim 256 wherein N1 comprises the sequence of the antisense strand of the first NARS.

263. The method of claim 256 wherein N1 comprises the sequence of the sense strand of the first NARS.

264. The method of claim 263 wherein both the first and the second NAs are restriction endonucleases (REs), and at least one of the nucleoside triphosphate(s) is modified.

265. The method of claim 256 wherein A1 is from 8 to 24 nucleotides in length.

266. The method of claim 265 wherein A1 is from 12 to 17 nucleotides in length.

267. The method of claim 256 wherein A2 is from 8 to 24 nucleotides in length.

268. The method of claim 267 wherein A2 is from 12 to 17 nucleotides in length.

269. The method of claim 256 wherein each of steps (B) and (D) is performed under isothermal conditions.

270. The method of claim 269 wherein each of steps (B) and (D) is performed at 50° C.-70° C.

271. The method of claim 256 wherein the DNA polymerase is 5′→3′ exonuclease deficient.

272. The method of claim 271 wherein the 5′→3′ exonuclease deficient DNA polymerase is selected from the group consisting of exo⁻ Vent, exo⁻ Deep Vent, exo⁻ Bst, exo⁻ Pfu, exo⁻ Bca, the Klenow fragment of DNA polymerase I, T5 DNA polymerase, Phi29 DNA polymerase, phage M2 DNA polymerase, phage PhiPRD1 DNA polymerase, Sequenase, PRD1 DNA polymerase, 9° Nm™ polymerase and T4 DNA polymerase homoenzyme.

273. The method of claim 272 wherein the 5′→3′ exonuclease deficient DNA polymerase is exo⁻ Bst polymerase, exo⁻ Bca polymerase, exo⁻ Vent polymerase, exo⁻ Deep Vent polymerase, or 9° Nm™ polymerase.

274. The method of claim 256 wherein the DNA polymerase has a strand displacement activity.

275. The method of claim 256 wherein each of steps (B) and (D) is performed in the presence of a strand displacement facilitator.

276. The method of claim 275 wherein the strand displacement facilitator is selected from the group consisting of BMRF1 polymerase accessory subunit, adenovirus DNA-binding protein, herpes simplex viral protein ICP8, single-stranded DNA binding proteins, phage T4 gene 32 protein, calf thymus helicase, and trehalose.

277. The method of claim 276 wherein the strand displacement facilitator is trehalose.

278. The method of claim 256 wherein step (E) is performed at least partially by the use of a technique selected from the group consisting of mass spectrometry, liquid chromatography, fluorescence polarization, and electrophoresis.

279. The method of claim 278 wherein step (E) is performed at least partially by the use of liquid chromatography.

280. The method of claim 278 wherein step (E) is performed at least partially by the use of mass spectrometry.

281. The method of claim 256 wherein the N1 is immobilized.

282. The method of claim 281 wherein the T2 is immobilized.

283. A method for determining the presence or absence of a junction between an upstream exon (Exon A) and a downstream exon (Exon B) of a gene in a cDNA molecule, comprising
(A) forming a mixture of a first oligonucleotide primer (ODNP), a second ODNP, and the cDNA molecule, wherein
(i) the first ODNP comprises a sequence that is at least substantially complementary to a portion of the antisense strand of Exon A near the 5′ terminus of Exon A in the antisense strand,
(ii) the second ODNP comprises a sequence that is at least substantially complementary to a portion of the sense strand of Exon B near the 5′ terminus of Exon B in the sense strand, and
(iii) at least one of the first ODNP and the second ODNP further comprises a sequence of a sense strand of a first nicking agent recognition sequence (NARS); and
(B) performing a first amplification reaction in the presence of a nicking agent (NA) that recognizes the first NARS under the conditions that amplify a first single-stranded nucleic acid (A1) if both Exon A and Exon B are present in the cDNA;
(C) providing a second single-stranded nucleic acid molecule (T2) comprising, from 5′ to 3′:
(i) a first sequence comprising
(a) a 3′ portion of the sense strand of Exon A linked at the 3′ terminus of the 3′ portion to a 5′ portion of the sense strand of Exon B at the 5′ terminus of the 5′ terminus, or
(b) a 5′ portion of the antisense strand of Exon A linked at the 5′ terminus of the 5′ portion to a 3′ portion of the antisense strand of Exon B at the 3′ terminus of the 3′ portion,
wherein if the cDNA contains the junction between Exon A and Exon B, the first sequence of the T2 is at least substantially complementary to the A1 molecule, but if the cDNA does not contain the junction between Exon A and Exon B, the T2 is not substantially complementary to the A1 molecule,
(ii) a sequence of the antisense strand of a second NARS, and
(iii) a second sequence;
(D) performing an amplification reaction that amplify a third single-stranded nucleic acid molecule (A2) using at least a portion of the second sequence of T2 as a template if the junction between Exon A and Exon B is present in the target cDNA molecule; and
(E) detecting the presence or absence of the A2 to determine the presence or absence of the junction in the cDNA molecule.

284. The method of claim 283 wherein the first NARS is identical to the second NARS.

285. The method of claim 283 wherein the cDNA molecule is immobilized.

286. The method of claim 283 wherein the first ODNP, the second ODNP or both are immobilized.

287. A method for determining the presence or absence of a junction between an upstream exon (Exon A) and a downstream exon (Exon B) of a gene in a cDNA molecule, comprising
(A) forming a mixture of a first oligonucleotide primer (ODNP), a second ODNP, and the cDNA molecule, wherein
(i) the first ODNP comprises
(a) a sequence that is at least substantially complementary to a portion of the antisense strand of Exon A near the 5′ terminus of Exon A in the antisense strand, and
(b) a sequence of the sense strand of a first nicking agent recognition sequence (NARS); and
(ii) the second ODNP comprises
(a) a sequence that is at least substantially complementary to a portion of the sense strand of Exon B near the 5′ terminus of Exon B in the sense strand, and
(b) a sequence of the sense strand of a second NARS;
(B) performing a first amplification reaction in the presence of a first nicking agent (NA) that recognizes the first NARS and a second NA that recognizes the second NARS under the conditions that amplify a first single-stranded nucleic acid (A1) if both Exon A and Exon B are present in the cDNA;
(C) providing a second single-stranded nucleic acid molecule (T2) comprising, from 5′ to 3′:
(i) a first sequence comprising
(a) a 3′ portion of the sense strand of Exon A linked at the 3′ terminus of the 3′ portion to a 5′ portion of the sense strand of Exon B at the 5′ terminus of the 5′ terminus, or
(b) a 5′ portion of the antisense strand of Exon A linked at the 5′ terminus of the 5′ portion to a 3′ portion of the antisense strand of Exon B at the 3′ terminus of the 3′ portion,
wherein if the cDNA contains the junction between Exon A and Exon B, the first sequence of the T2 is at least substantially complementary to the A1 molecule, but if the cDNA does not contain the junction between Exon A and Exon B, the T2 is not substantially complementary to the A1 molecule,
(ii) a sequence of the antisense strand of a second NARS, and
(iii) a second sequence;
(D) performing an amplification reaction that amplify a third single-stranded nucleic acid molecule (A2) using at least a portion of the second sequence of T2 as a template if the junction between Exon A and Exon B is present in the target cDNA molecule; and
(E) detecting the presence or absence of the A2 to determine the presence or absence of the junction in the cDNA molecule.

288. The method of 287 wherein the first, second and third NARS are identical.

289. The method of claim 287 wherein the cDNA molecule is immobilized.

290. The method of claim 287 wherein the first ODNP, the second ODNP or both are immobilized.

291. A method for amplifying one or more single-stranded nucleic acids, comprising:
(a) applying to the array of claim 188
(i) one or more nucleic acid amplification reaction mixtures, wherein the amplification reaction was performed in the presence of a first nicking agent, or
(ii) the amplification product(s) of the amplification reaction of (i); and
(b) performing an amplification reaction on the array in the presence of a second nicking agent that recognizes the nicking agent recognition sequence of which the antisense strand is present in the isolated nucleic acid molecules immobilized to the substrate of the array to amplify one or more single-stranded nucleic acids.

292. The method of claim 291 wherein the first nicking agent is identical to the second nicking agent.
